SEQUENCE LISTING (41 sequences)

SEQ ID NO: 1 - protein, 326 aa, Yersinia pestis
MIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIKAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGK

SEQ ID NO: 2 - DNA, 981 bp, Yersinia pestis
atgattagagcctacgaacaaaacccacaacattttattgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatccatgataaatccattaatctcatggataaaaatttatatggttatacagatgaagagatttttaaagccagcgcagagtacaaaattctcgagaaaatgcctcaaaccaccattcaggtggatgggagcgagaaaaaaatagtctcgataaaggactttcttggaagtgagaataaaagaaccggggcgttgggtaatctgaaaaactcatactcttataataaagataataatgaattatctcactttgccaccacctgctcggataagtccaggccgctcaacgacttggttagccaaaaaacaactcagctgtctgatattacatcacgttttaattcagctattgaagcactgaaccgtttcattcagaaatatgattcagtgatgcaacgtctgctagatgacacgtctggtaaatga

SEQ ID NO: 3 - protein, 326 aa, Yersinia pseudotuberculosis
MIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVIHFSLTADRIDDDILKVIVDSMNHHGDARSKLREELAELTAELKIYSVIQAEINKHLSSGGTINIHDKSINLMDKNLYGYTDEEIFKASAEYKILEKMPQTTIQEGETEKKIVSIKNFLESEKKRTGALGNLKDSYSYNKDNNELSHFATTCSDKSRPLNDLVSQKTTQLSDITSRFNSAIEALNRFIQKYDSVMQRLLDDTSGK

SEQ ID NO: 4 - DNA, 981 bp, Yersinia pseudotuberculosis
atgattagagcctacgaacaaaacccacaacattttattgaggatctagaaaaagttagggtggaacaacttactggtcatggttcttcagttttagaagaattggttcagttagtcaaagataaaaatatagatatttccattaaatatgatcccagaaaagattcggaggtttttgccaatagagtaattactgatgatatcgaattgctcaagaaaatcctagcttattttctacccgaggatgccattcttaaaggcggtcattatgacaaccaactgcaaaatggcatcaagcgagtaaaagagttccttgaatcatcgccgaatacacaatgggaattgcgggcgttcatggcagtaatacatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtggtggcaccataaatatccatgataaatcaattaatctcatggataaaaatttatatggttatacagatgaagagatttttaaagccagtgcagagtacaaaattctcgagaaaatgcctcaaaccaccattcaggaaggtgagaccgaaaaaaaaatagtctcgataaagaactttcttgaaagtgagaaaaaaagaaccggggcgttgggtaatctgaaagactcatactcttataataaagataataatgaattatctcactttgccaccacctgctcggataagtccaggccgctcaacgacttggttagccaaaaaacaactcagctgtctgatattacatcacgttttaattcagctattgaagcactgaaccgtttcattcagaaatatgattcagtgatgcaacgtctgctagatgacacgtctggtaaatga

SEQ ID NO: 5 - protein, 326 aa, Yersinia pestis
MIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGK

SEQ ID NO: 6 - DNA, 981 bp, Yersinia pestis; identical to SEQ ID NO: 2

SEQ ID NO: 7 - protein, 334 aa, Yersinia enterocolitica
MIRAYEQNPQHFIEDLEKVRVDQLTGHGSSVLEELVQLVKDKKIDISIKYDPKKDSEVFAERVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHEKSINLMDKNLYGYTDEEIFKASAEYKILKKMPQTTIKDDELHEVGVIAGAEKQIVSIKNFLESENKRTGALGNLKDSYSYNKDNNELSHFATACSDKSRPLNDLVSQKTTQLSDITSRFNSAIEALNRFIQKYDSVMQRLLDDTRL

SEQ ID NO: 8 - DNA, 1002 bp, Yersinia enterocolitica
atgattagagcctacgaacaaaacccacaacattttattgaggatctagaaaaagttagggtggaacaacttactggtcatggttcttcagttttagaagaattggttcagttagtcaaagataaaaagatagatatttccattaaatatgatcccaaaaaagattcggaggtttttgccgaaagggtaattactgatgatatcgaattactcaagaaaatcctggcttattttctacccgaggatgccattcttaaaggcggtcattatgacaaccaactgcaaaatggcatcaagcgagtaaaagagttccttgaatcatcgccgaatacacaatgggagttgcgggcgttcatggcagtaatgcatttctctttaaccgccgatcgtatcgatgatgatattttgaaagtgattgttgattcaatgaatcatcatggtgatgcccgtagcaagttgcgtgaagaattagctgagcttaccgccgaattaaagatttattcagttattcaagccgaaattaataagcatctgtctagtagtggcaccataaatatacatgagaaatcaattaatctcatggataaaaatttatatggttatacagatgaagagatttttaaagccagcgcagagtacaaaattctcaagaaaatgcctcaaaccaccattaaggacgacgagttgcacgaggttggagttatagctggggccgaaaaacaaatagtctcgataaagaactttcttgaaagtgagaataaaagaaccggggcgttgggtaatctgaaagattcatactcttataataaagataataatgaattatctcactttgccaccgcctgctcggataagtccaggccgctcaacgacttggttagccaaaaaacaactcagctgtctgatattacatcacgttttaattcagctattgaagcactgaaccgtttcattcagaaatatgattcagtaatgcaacgtctgctagatgacacgaggtta

SEQ ID NO: 9 - protein, 312 aa, Yersinia pestis
MKKSSIVATIITILSGSANAASSQLIPNISPDSFTVAASTGMLSGKSHEMLYDAETGRKISQLDWKIKNVAILKGDISWDPYSFLTLNARGWTSLASGSGNMDDYDWMNENQSEWTDHSSHPATNVNHANEYDLNVKGWLLQDENYKAGITAGYQETRFSWTATGGSYSYNNGAYTGNFPKGVRVIGYNQRFSMPYIGLAGQYRINDFELNALFKFSDWVRAHDNDEHYMRDLTFREKTSGSRYYGTVINAGYYVTPNAKVFAEFTYSKYDEGKGGTQTIDKNSGDSVSIGGDAAGISNKNYTVTAGLQYRF

SEQ ID NO: 10 - DNA, 939 bp, Yersinia pestis
atgaagaaaagttctattgtggcaaccattataactattctgtccgggagtgctaatgcagcatcatctcagttaataccaaatatatcccctgacagctttacagttgcagcctccaccgggatgctgagtggaaagtctcatgaaatgctttatgacgcagaaacaggaagaaagatcagccagttagactggaagatcaaaaatgtcgctatcctgaaaggtgatatatcctgggatccatactcatttctgaccctgaatgccagggggtggacgtctctggcttccgggtcaggtaatatggatgactacgactggatgaatgaaaatcaatctgagtggacagatcactcatctcatcctgctacaaatgttaatcatgccaatgaatatgacctcaatgtgaaaggctggttactccaggatgagaattataaagcaggtataacagcaggatatcaggaaacacgtttcagttggacagctacaggtggttcatatagttataataatggagcttataccggaaacttcccgaaaggagtgcgggtaataggttataaccagcgcttttctatgccatatattggacttgcaggccagtatcgcattaatgattttgagttaaatgcattatttaaattcagcgactgggttcgggcacatgataatgatgagcactatatgagagatcttactttccgtgagaagacatccggctcacgttattatggtaccgtaattaacgctggatattatgtcacacctaatgccaaagtctttgcggaatttacatacagtaaatatgatgagggcaaaggaggtactcagaccattgataagaatagtggagattctgtctctattggcggagatgctgccggtatttccaataaaaattatactgtgacggcgggtctgcaatatcgcttctga

SEQ ID NO: 11 - protein, 673 aa, Yersinia pestis
MKMTRLYPLALGGLLLPAIANAQTSQQDESTLVVTASKQSSRSASANNVSSTVVSAPELSDAGVTASDKLPRVLPGLNIENSGNMLFSTISLRGVSSAQDFYNPAVTLYVDGVPQLSTNTIQALTDVQSVELLRGPQGTLYGKSAQGGIINIVTQQPDSTPRGYIEGGVSSRDSYRSKFNLSGPIQDGLLYGSVTLLRQVDDGDMINPATGSDDLGGTRASIGNVKLRLAPDDQPWEMGFAASRECTRATQDAYVGWNDIKGRKLSISDGSPDPYMRRCTDSQTLSGKYTTDDWVFNLISAWQQQHYSRTFPSGSLIVNMPQRWNQDVQELRAATLGDARTVDMVFGLYRQNTREKLNSAYDMPTMPYLSSTGYTTAETLAAYSDLTWHLTDRFDIGGGVRFSHDKSSTQYHGSMLGNPFGDQGKSNDDQVLGQLSAGYMLTDDWRVYTRVAQGYKPSGYNIVPTAGLDAKPFVAEKSINYELGTRYETADVTLQAATFYTHTKDMQLYSGPVRMQTLSNAGKADATGVELEAKWRFAPGWSWDINGNVIRSEFTNDSELYHGNRVPFVPRYGAGSSVNGVIDTRYGALMPRLAVNLVGPHYFDGDNQLRQGTYATLDSSLGWQATERMNISVYVDNLFDRRYRTYGYMNGSSAVAQVNMGRTVGINTRIDFF

SEQ ID NO: 12 - DNA, 2022 bp, Yersinia pestis
atgaaaatgacacggctttatcctctggccttggggggattattgctccccgccattgctaatgcccagacttcacagcaagacgaaagcacgctggtggttaccgccagtaaacaatcttcccgctcggcatcagccaacaacgtctcgtctactgttgtcagcgcgccggaattaagcgacgccggcgtcaccgccagcgacaaactccccagagtcttgcccgggctcaatattgaaaatagcggcaacatgcttttttcgacgatctcgctacgcggcgtctcttcagcgcaggacttctataaccccgccgtcaccctgtatgtcgatggcgtccctcagctttccaccaacaccatccaggcgcttaccgatgtgcaaagcgtggagttgctgcgaggcccacagggaacgttatatggcaaaagcgctcagggcgggatcatcaacatcgtcacccagcagccggacagcacgccgcgcggctatattgaaggcggcgtcagtagccgcgacagttatcgaagtaagttcaacctgagcggccccattcaggatggcctgctgtacggcagcgtcaccctgttacgccaggttgatgacggcgacatgattaaccccgcgacgggaagcgatgacttaggcggcacccgcgccagcatagggaatgtgaaactgcgtctggcgccggacgatcagccctgggaaatgggctttgccgcctcacgcgaatgtacccgcgccacccaggacgcctatgtgggatggaatgatattaagggccgtaagctgtcgatcagcgatggttcaccagacccgtacatgcggcgctgcactgacagccagaccctgagtgggaaatacaccaccgatgactgggttttcaacctgatcagcgcctggcagcagcagcattattcgcgcaccttcccttccggttcgttaatcgtcaatatgcctcagcgctggaatcaggatgtgcaggagctgcgcgccgcaaccctgggcgatgcgcgtaccgttgatatggtgtttgggctgtaccggcagaacacccgcgagaagttaaattcagcctacgacatgccgacaatgccttatttaagcagtaccggctataccaccgctgaaacgctggccgcatacagtgacctgacctggcatttaaccgatcgttttgatatcggcggcggcgtgcgcttctcgcatgataaatccagtacacaatatcacggcagcatgctcggcaacccgtttggcgaccagggtaagagcaatgacgatcaggtgctcgggcagctatccgcaggctatatgctgaccgatgactggagagtgtatacccgtgtagcccagggatataaaccttccgggtacaacatcgtgcctactgcgggtcttgatgccaaaccgttcgtcgccgagaaatccatcaactatgaacttggcacccgctacgaaaccgctgacgtcacgctgcaagccgcgacgttttatacccacaccaaagacatgcagctttactctggcccggtcaggatgcagacattaagcaatgcgggtaaagccgacgccaccggcgttgagcttgaagcgaagtggcggtttgcgccaggctggtcatgggatatcaatggcaacgtgatccgttccgaattcaccaatgacagtgagttgtatcacggtaaccgggtgccgttcgtaccacgttatggcgcgggaagcagcgtgaacggcgtgattgatacgcgctatggcgcactgatgccccgactggcggttaatctggtcgggccgcattatttcgatggcgacaaccagttgcggcaaggcacctatgccaccctggacagcagcctgggctggcaggcgactgaacggatgaacatttccgtctatgtcgataacctgttcgaccgtcgttaccgtacctatggctacatgaacggcagcagcgccgtcgcgcaggtcaatatgggtcgcaccgtcggtatcaatacgcgaattgatttcttctga

SEQ ID NO: 13 - protein, 20 aa, Yersinia sp.
VLEELVQLVKDKNIDISIKY

SEQ ID NO: 14 - protein, 20 aa, Yersinia sp.
INLMDKNLYGYTDEEIFKAS

SEQ ID NO: 15 - protein, 20 aa, Artificial Sequence
  Description of Artificial Sequence Synthetic polypeptide
VLEELVQLVKDKNIDISIKY

SEQ ID NO: 16 - protein, 20 aa, Artificial Sequence
  Description of Artificial Sequence Synthetic polypeptide
INLMDKNLYGYTDEEIFKAS

SEQ ID NO: 17 - protein, 100 aa, Artificial Sequence
  Description of Artificial Sequence Synthetic polypeptide
VLEELVQLVKDKNIDISIKYVLEELVQLVKDKNIDISIKYVLEELVQLVKDKNIDISIKYVLEELVQLVKDKNIDISIKYVLEELVQLVKDKNIDISIKY

SEQ ID NO: 18 - protein, 100 aa, Artificial Sequence; identical to SEQ ID NO: 17
  Description of Artificial Sequence Synthetic polypeptide

SEQ ID NO: 19 - protein, 100 aa, Artificial Sequence
  Description of Artificial Sequence Synthetic polypeptide
INLMDKNLYGYTDEEIFKASINLMDKNLYGYTDEEIFKASINLMDKNLYGYTDEEIFKASINLMDKNLYGYTDEEIFKASINLMDKNLYGYTDEEIFKAS

SEQ ID NO: 20 - protein, 100 aa, Artificial Sequence; identical to SEQ ID NO: 19
  Description of Artificial Sequence Synthetic polypeptide

SEQ ID NO: 21 - protein, 100 aa, Artificial Sequence; identical to SEQ ID NO: 17
  Description of Artificial Sequence Synthetic polypeptide

SEQ ID NO: 22 - protein, 100 aa, Artificial Sequence; identical to SEQ ID NO: 19
  Description of Artificial Sequence Synthetic polypeptide

SEQ ID NO: 23 - protein, 40 aa, Yersinia sp.
VLEELVQLVKDKNIDISIKYINLMDKNLYGYTDEEIFKAS

SEQ ID NO: 24 - protein, 40 aa, Yersinia sp.
INLMDKNLYGYTDEEIFKASVLEELVQLVKDKNIDISIKY

SEQ ID NO: 25 - protein, 80 aa, Yersinia sp.
VLEELVQLVKDKNIDISIKYINLMDKNLYGYTDEEIFKASVLEELVQLVKDKNIDISIKYINLMDKNLYGYTDEEIFKAS

SEQ ID NO: 26 - protein, 60 aa, Yersinia sp.
INLMDKNLYGYTDEEIFKASVLEELVQLVKDKNIDISIKYINLMDKNLYGYTDEEIFKAS

SEQ ID NO: 27 - protein, 40 aa, Artificial Sequence; identical to SEQ ID NO: 23
  Description of Artificial Sequence Synthetic polypeptide

SEQ ID NO: 28 - protein, 40 aa, Artificial Sequence; identical to SEQ ID NO: 24
  Description of Artificial Sequence Synthetic polypeptide

SEQ ID NO: 29 - protein, 30 aa, Artificial Sequence
  Description of Artificial Sequence Synthetic polypeptide
ATGGSYSYNNGAYTGNFPKGVRVIGYNQRF

SEQ ID NO: 30 - protein, 20 aa, Artificial Sequence
  Description of Artificial Sequence Synthetic polypeptide
RAHDNDEHYMRDLTFREKTS

SEQ ID NO: 31 - protein, 28 aa, Artificial Sequence
  Description of Artificial Sequence Synthetic polypeptide
KGGTQTIDKNSGDSVSIGGDAAGISNKN

SEQ ID NO: 32 - protein, 13 aa, Artificial Sequence
  Description of Artificial Sequence Synthetic consensus polypeptide
VLEELXXXXXDKN

SEQ ID NO: 33 - protein, 13 aa, Artificial Sequence
  Description of Artificial Sequence Synthetic consensus polypeptide
DKNXXXXTDEEIF

SEQ ID NO: 34 - protein, 20 aa, Artificial Sequence
  Description of Artificial Sequence Synthetic peptide
VLEELVQLVKDKNIDISIDY

SEQ ID NO: 35 - protein, 18 aa, Yersinia pestis
LMDKNLYGYTDEEIFKAS

SEQ ID NO: 36 - protein, 27 aa, Artificial Sequence
  Description of Artificial Sequence Synthetic peptide
FNPSESDVVSELGKVETVTIRRLHIPQ

SEQ ID NO: 37 - protein, 12 aa, Artificial Sequence
  Description of Artificial Sequence Synthetic peptide
SQMLFRGRRASQ

SEQ ID NO: 38 - protein, 4 aa, Artificial Sequence
  Description of Artificial Sequence Synthetic peptide motif
LEEL

SEQ ID NO: 39 - protein, 4 aa, Artificial Sequence
  Description of Artificial Sequence Synthetic peptide motif
DEEI

SEQ ID NO: 40 - protein, 4 aa, Artificial Sequence
  Description of Artificial Sequence Synthetic peptide motif
IRRL

SEQ ID NO: 41 - protein, 4 aa, Artificial Sequence
  Description of Artificial Sequence Synthetic peptide motif
GRRA

(19) United States
(12) Patent Application Publication
(10) Pub. No.: US 2006/0246084 A1
(43) Pub. Date: Nov. 2, 2006

(54) Yersinia polypeptide vaccines, antibodies and immunomodulatory proteins

(75) Inventors: Robert R. Brubaker, Vermontville, MI (US); Vyacheslav M. Abramov, Lyubuchany (RU)

Correspondence Address: WILMER CUTLER PICKERING HALE AND DORR LLP, 60 STATE STREET, BOSTON, MA, 02109, US

(73) Assignee: Michigan State University

(21) Appl. No.: 11/403,533

(22) Filed: Apr. 12, 2006

Related U.S. Application Data
(63) Continuation-in-part of application No. 11/089,158, filed on Mar. 24, 2005.
Application No. 11/089,158 is a continuation of application No. 10/694,614, filed on Oct. 27, 2003, now Pat. No. 6,964,770.
Application No. 10/694,614 is a division of application No. 08/302,423, filed on Sep. 8, 1994, now Pat. No. 6,638,510.
(60) Provisional application No. 60/670,771, filed on Apr. 12, 2005.

(51) Int. Cl. A61K 39/00; A61K 39/38

(52) U.S. Cl. 424/184.1

(57) ABSTRACT

Disclosed are compositions, including LcrV antigenic polypeptides, vaccines and antibodies, as well as associated methods for treating and/or preventing Yersinia infection in a host. The invention further provides immunomodulatory LcrV proteins and polypeptides comprising TLR2 and IFN-γR-IFN-γ-binding sequences that stimulate host anti-inflammatory responses and repress pro-inflammatory responses.

### RELATED APPLICATIONS

This application claims the benefit of U.S. Provisional Application No. 60/670,771, and is a continuation-in-part of U.S. application Ser. No. 11/089158, filed on Mar. 24, 2005, which is a continuation of U.S. application Ser. No. 10/694614, filed on Oct. 27, 2003, now U.S. Pat. No. 6,964,770, which is a divisional of U.S. application Ser. No. 08/302,423, filed Sep. 8, 1994, now U.S. Pat. No. 6,638,510, the contents of each of which are incorporated herein in their entireties.

### GOVERNMENT SUPPORT

The United States government may have certain rights to this application in accordance with U.S. Public Health Service Grant AI 19353, Regional Centers of Excellence for Biodefense and Emerging Infectious Disease Research (RCE) NIH grant 1-U54-AI-057153, and International Science and Technology Center (ISTC) grant #2069 (U.S. Department of Defense and European Union).

### FIELD OF THE INVENTION

This invention relates to the field of medical science. In particular, this invention relates to the treatment and prevention of infectious disease, particularly bubonic plague.

### 1. BACKGROUND OF THE INVENTION

Plague is an infectious disease caused by the bacteria Yersinia pestis, which is a non-motile, slow-growing facultative organism in the family Enterobacteriacea. Y. pestis is carried by rodents, particularly rats, and in the fleas that feed on them. Other animals and humans usually contract the bacteria directly from rodent or flea bites.

Yersinia pestis is found in animals throughout the world, most commonly in rats but occasionally in other wild animals, such as prairie dogs. Most cases of human plague are caused by bites of infected animals or the infected fleas that feed on them. Y. pestis can affect people in three different ways, and the resulting diseases are referred to as bubonic plague, septicemic plague, and pneumonic plague.

In bubonic plague, which is the most common form of Y. pestis-induced disease, bacteria infect the lymph system, which becomes inflamed. Bubonic plague is typically contracted by the bite of an infected flea or rodent. In rare cases, Y. pestis bacteria, from a piece of contaminated clothing or other material used by a person with plague, enter through an opening in your skin. Bubonic plague affects the lymph nodes, and within three to seven days of exposure to the bacteria, flu-like symptoms will develop such as fever, headache, chills, weakness, and swollen, tender lymph glands (called buboes—hence the name bubonic). Bubonic plague is rarely spread from person to person.

Septicemic plague is contracted the same way as bubonic plague-usually through a flea or rodent bite when the bacteria multiply in the blood, but is characterized by the occurrence of multiplying bacteria in the bloodstream, rather than just the lymph system. Septicemic plague usually occurs as a complication of untreated bubonic or pneumonic plague, and its symptoms include fever, chills, weakness, abdominal pain, shock, and bleeding underneath the skin or other organs. Buboes, however, do not develop in septicemic plague, and septicemic plague is rarely spread from person to person.

Pneumonic plague is the most serious form of plague and occurs when Y. pestis bacteria infect the lungs and cause pneumonia. Pneumonic plague can be contracted when Y. pestis bacteria are inhaled. Within one to three days of exposure to airborne droplets of pneumonic plague, fever, headache, weakness, rapid onset of pneumonia with shortness of breath, chest pain, cough, and sometimes bloody or watery sputum develop. This type of plague can also be spread from person to person when bubonic or septicemic plague goes untreated after the disease has spread to the lungs. At this point, the disease can be transmitted to someone else by Y. pestis-carrying respiratory droplets that are released into the air when the infected individual coughs.

Virulence factors of Yersinia pestis are encoded on the chromosome (e.g., iron transport functions and antigen 4), a 100-kb toxin or Tox plasmid (murine exotoxin and capsular fraction 1 antigen), a 70-kb low-calcium response or Lcr plasmid (yersinia outer membrane peptides termed Yops), and a 10-kb pesticin or Pst plasmid (plasminogen activator). The enteropathogenic yersiniae (Yersinia pseudotuberculosis and Yersinia enterocolitica) possess only the Lcr plasmid and thus lack a number of determinants necessary for expression of severe systemic disease (Brubaker et al. (1991) Clin. Microbiol. Rev. 4:309-324). The common Lcr plasmid mediates restriction of growth at 37° C. unless the environment contains mammalian extracellular levels of Ca²⁺ (2.5 mM). Cells of Y. pestis arrested in this physiological state fail to synthesize stable RNA (Charnetzky et al. (1982) J. Bacteriol. 149:1089-1095) or bulk vegetative protein (Mehigh et al. (1993) Infect. Immun. 61:13-22; Mehigh et al. (1989) Microb. Pathog. 6:203-217; Zahorchak et al. (1979) J. Bacterial. 39:792-799), but either continue synthesis or induce expression of stress functions (e.g., GroEL-like protein) as well as most of the virulence factors noted above (Lcr⁺) (Mehigh et al. (1993) Infect. lmmun. 61:13-22).

The Yops encoded by the Lcr plasmid are secreted peptides that, in Y. pestis, can be distinguished in vitro by whether they are released intact into culture supernatant fluids or appear as small fragments after undergoing posttranslational degradation (Sample et al. (1987) Microb. Pathog. 3:239-248; Sample et al. (1987) Microb. Pathog. 2:443-453) mediated by the species-specific plasminogen activator (Sodeinde et al. (1988) Infect. lmmun. 56:2749-2752). Export in vitro of degradable Yops in intact form by Pst plasmid-deficient enteropathogenic yersiniae is promoted by Lcr plasmid-encoded functions (Michiels et al. (1991) J. Bacteriol. 173:1677-1685; Michiels et al. (1991) J. Bacteriol. 173:4994-5009) involving evident recognition without processing of the N-terminal end of these peptides (see, e.g., Michiels et al. (1990) Infect. lmmun. 58:2840-2849). Similarly, the stable (i.e., non-degradable) YopM of Y. pestis is secreted from the bacterium with an intact N terminus (Reisner et al. (1992) Infect. Immun. 60:5242-5252). Degradable Yops E (see, e.g., Rosqvist et al. (1990) Mol. Microbial. 4:657-667; Yops K plus YopsL (Straley et al. (1989) Infect. Immun. 57:1200-1210), and probably YopsB (Hakaussou et al. (1993) Infect. Immun. 61:71-80), as well as stable YopM (see, e.g., Leung et al. (1990) Infect. Immun. 58:3262-3270), are all established virulence factors. YpkA also belongs in this category (Galyov et al. (1993) Nature 361:730-732). All of these degradable peptides possess properties consistent with roles as cytotoxins (see, e.g., Rosqvist et al. (1990) Mol. Microbial. 4:657-667;), whereas YopM binds to thrombin and might thus function in concert with plasminogen activator during terminal disease (Sodeinde et al. (1992) Science 258:1004-1007) to promote hemorrhagic sequelae (Leung et al. (1990) Infect. Immun. 58:3262-3271; Leung et al. (1989) J. Bacteriol. 171:4623-4632; Reisner et al. (1992) Infect. Immun. 60:5242-5252). Degradable YopD may serve to deliver cytotoxic Yops to target cells (Hakaussou et al. (1993) Infect. Immun. 61:71-80; Rosqvist et al. (1991) Infect. Immun. 59:4562-4569), and stable YopN was assigned a role in sensing Ca²⁺ (Forsberg et al. (1991) Mol. Microbiol. 5:977986).

A putative virulence factor encoded by the Lcr plasmid (Perry et al. (1986) Infect. Immun. 54:428-434) is LcrV (V antigen), initially described as a major exported peptide of wild-type Y. pestis (Burrows (1956) Nature 177:426-427; Burrows et al. (1956) Br. J. Exp. Pathol. 37:481-493) and Y. pseudotuberculosis (Burrows et al. (1960) Br. J. Exp. Pathol. 41:38-44) and later identified in Lcr⁺ isolates of Y. enterocolitica (Carter et al. (1980) Infect. Immun. 28:638-640; Perry et al. (1983) Infect. Immun. 40:166-171). Results of genetic analysis positioned LcrV within an IcrGVH-yopBD operon (see, e.g., Bergman et al. (1991) J. Bacteriol. 173:1607-1616; Price et al. (1989) J. Bacterial. 171:5646-5653) and showed that a nonpolar deletion in lcrV promoted loss of the nutritional requirement for Ca²⁺ and resulted in a virulence (Hakaussou et al. (1993) Infect. Immun. 61:71-80; Price et al. (1991) J. Bacterial. 73:2649-2657). The V antigen (LcrV) of Yersinia pestis was implicated as a major determinant of virulence upon its discovery in 1956. Specific antibodies raised against crude preparations were also found to be immunogenic (Burrows et al. (1958) Brit. J. Exp. Pathol., 39:278-291; Lawton et al. (1963) J. Immunol., 91:179-184) but, due to the extraordinary liability of this 37.3-kDa protein (see, e.g., Brubaker et al. (1987) Microb. Pathogen, 2:49-62; formal proof of protection required rapid isolation of a homogenous staphylococcal Protein A-LcrV fusion protein by affinity chromatography (Motin et al. (1994) Infect. Immun., 62:4192-4201). A similar hexahistidine-tagged LcrV fusion actively immunized mice against intravenous challenge with 10⁷ plague bacilli assuring a protective role for at least one internal epitope located between amino acids 168-275 (Motin et al. (1994) Infect. Immun., 62:4192-4201; Motin et al. (1996) Infect. Immun., 64:4313-4318). LcrV is encoded within an lcrGVH-yopBD operon of pCD (Perry et al. (1986) Infect. Immun., 54:428-434), a 70.5-kb virulence plasmid (Ferber et al. (1981) Infect. Immun., 31:839-841) that mediates a type III secretion system (TTSS) capable of translocating essential virulence effectors termed Yops into host cell cytoplasm (Mota et al. (2005) Ann. Med., 37:234-249).

LcrV was reported to regulate expression of Yops (see, e.g., Bergman et al. (1991) J. Bacteriol., 173:1607-1616; promote assembly of the TTSS injectisome (Goure et al. (2005) J. Infect. Dis., 192:218-225; Sarker et al. (1998) J. Bacteriol., 180:1207-1214), serve as an integral component of the Yop injectisome (see, e.g., Mueller et al. (2005) Science, 310:674-676), and target host cells for delivery of Yops (Lee et al. (2000) J. Biol. Chem., 275:36869-36875). LcrV is secreted abundantly at 37° C. into Ca²⁺-deficient culture media (Lawton et al. (1963) J. Immunol., 91:179-184) and in vivo (Smith et al. (1960) Br. J. Exp. Pathol., 41:452-459) where it blocks innate immunity by upregulating the major anti-inflammatory cytokine interleukin-10 (IL-10) (Nedialkov et al. (1997) Infect. Immun., 65:1196-1203). The latter removes NF-κB from host cell nuclei thereby downregulating numerous proinflammatory functions including cytokines required for activation of professional phagocytes (Moore et al. (2001) Ann. Rev. Immunol., 19:683-765).

Amplification of IL-10 by LcrV requires co-expression of Toll-like receptor-2 (TLR-2) plus the differentiation factor CD14 and fails to occur in IL-10^{-/-} knockout mice, which are highly resistant to infection (Reithmeier-Rost et al. (2004) Cell Immunol., 231:63-74; Sing et al. (2002) J. Immunol., 168:1315-1321; Sing et al. (2002) J. Exp. Med., 196:1017-1024). This mechanism is distinct from anti-inflammatory processes reported for YopJ (Viboud et al. (2004) Annu. Rev. Microbiol., 59:69-89; Zhang et al. (2005) J. Immunol., 174:7939-7949), which is not required for expression of virulence in Y. pestis (Goguen et al. (1984) J. Bacteriol., 160:842-848), and YopH (Bruckner et al. (2004) J. Biol. Chem., 280:10388-10394) that promote apoptosis or otherwise prevent net generation of nuclear NF-κB. Anti-LcrV might serve as an opsonin (see, e.g., Cowan et al. (2005) Infect. Immun., 73:6127-6137; prevent translocation of Yops (see, e.g., Mueller et al. (2005) Science, 310:674-676; or block amplification of IL-10 (Overheim et al. (2005) Infect. Immun., 73:5152-5159). Any one of these activities could promote immunity by preventing the ability of LcrV to block migration of neutrophils (Welkos et al. (1998) Microb. Pathol., 24:185-196) and inflammatory cells to infectious foci where they generate protective granulomas (see, e.g., Nakajima et al. (1995) Infect. Immun., 63:3021-3029.

Historically, plague has destroyed entire civilizations. In the 1300s, the “Black Death,” as it was called, killed approximately one-third (20 to 30 million) of Europe's population. In the mid-1800s, it killed 12 million people in China. Even today, with better living conditions, antibiotics, and improved sanitation available, current World Health Organization statistics show there were 2,118 cases of plague in the year 2003 worldwide. Worldwide, there have been small plague outbreaks in Asia, Africa, and South America. Approximately 10 to 20 people in the United States develop plague each year from flea or rodent bites-primarily from infected prairie dogs—in rural areas of the southwestern United States. About one in seven of those infected die from the disease. There is also renewed concern about Yersinia pestis as an agent of bioterrorism. Bioterrorism is a real threat to the United States and around the world, and, although the United States does not currently expect a plague attack, it is possible that pneumonic plague could occur via an aerosol distribution. Indeed, the Y. pestis bacterium is widely available in microbiology banks around the world, and thousands of scientists have worked with plague, making a biological attack a serious concern.

Killed whole vaccines against Yersinia pestis have been used since the 1890s (Williamson, (2001) J. Appl. Microbiol., 91:606-608). The whole-cell killed vaccine previously was available for people at possible high risk of exposure, such as military or laboratory personnel. Side effects were common, and multiple boosters were necessary. It also was unclear how well this vaccine protected against the pneumonic form of plague (Smego et al. (1999) Eur. J. Clin. Microbiol. Infect. Dis., 18:1-15). Therefore, production of the vaccine was discontinued by the manufacturer in 1999 (Inglesby et al. (2000) JAMA, 283:2281-2290) A live attenuated vaccine, EV76, also was in use in humans in some areas of the world, but it also is not commercially available (Williamson, (2001) J. Appl. Microbiol., 91:606-608). Previous experiments in mice revealed that purified F1 antigen was more effective in protecting against plague than the killed whole-cell vaccine (Friedlander et al. (1995) Clin. Infect. Dis., 21:S178-S181). However, attempts to develop a vaccine using only the F1 antigen were less than fully successful (Clin. Infect. Dis., 21:S178-S181).

Left untreated, bubonic plague bacteria can quickly multiply in the bloodstream, causing septicemic plague, or even progress to the lungs, causing pneumonic plague. Antibiotics are the primary treatment option currently available for treating and preventing bubonic, septicemic, and pneumonic plague. When the disease is suspected and diagnosed early, a health care provider can prescribe specific antibiotics, typically streptomycin or gentamycin, as treatment options. Certain other antibiotics are also effective. Notably, as described above, effective commercial vaccines against plague are not readily available commercially. Accordingly, new and enhanced immunological compositions and methods for combating Yersinia infection and disease would be useful in preventing new outbreaks as well as in treating diseased individuals.

### 2. SUMMARY OF THE INVENTION

The invention is based, in part upon the finding that antisera raised against recombinant V antigen or a stable staphylococcal protein A-V antigen fusion peptide (P A V) (which can be purified to homogeneity in one step by immunoglobulin G [IgG] affinity chromatography) can provide statistically significant protection against 10 minimum lethal doses of Y. pestis and Y. pseudotuberculosis but not Y. enterocolitica. The invention is further based upon the finding that this passive immunity is mediated by at least one internal protective epitope as shown by the absorption of anti-PAV with an excess of progressively smaller truncated derivatives of V antigen. Accordingly, the invention provides immunoprotective V antigen polypeptide fragments, including amino-terminally truncated, carboxy-terminally truncated LcrV proteins and LcrV polypeptide fragments, that are useful as Yersinia vaccines and for raising antibodies that provide passive resistance to Yersinia infection.

The invention is still further based upon the finding that LcrV possesses two non-cooperative binding domains capable of recognizing both free TLR-2 and IFN-γ bound to its receptor (IFN-γR-IFN-γ)—an N-terminal region spanning amino acids 31-50 of LcrV and a downstream site spanning amino acids 193-210 which also functions within the native LcrV molecule—to upregulate IL-10, downregulate LPS-induced TNF-α, and prevent oxidative killing by neutrophils. Accordingly, the invention provides LcrV proteins and polypeptides having these TLR-2 and IFN-γR-IFN-γ binding activities that upregulate major host anti-inflammatory cytokines including Interleukin-10 (IL-10), which, in turn, block the ability of host nuclear NF-κB to activate transcription of a plethora of inflammatory activities including proinflammatory cytokines. The invention thereby provides methods of blocking innate immunity and thereby facilitating allograft retention (preventing graft rejection), as well as methods of treating and preventing certain infectious diseases, such as HIV, and cancers.

In one aspect, the invention provides immunogenic recombinant LcrV polypeptides substantially free of antigenic contaminating proteins. In certain embodiments, the immunogenic LcrV polypeptide substantially free of antigenic contaminating protein consists essentially of an LcrV polypeptide fragment, or a carboxy-terminal or amino-terminal truncation of a Yersinia LcrV protein. In particular embodiments, the immunogenic carboxy-terminal or amino-terminal LcrV polypeptide truncation is fused to purification tag. In further particular embodiments, the purification tag is Protein A. In further embodiments, the immunogenic LcrV polypeptide substantially free of antigenic contaminating proteins includes an antigenic polypeptide sequence of the 259 carboxy-terminal amino acids of V antigen. In another embodiment, the immunogenic LcrV polypeptide substantially free of antigenic contaminating proteins consists essentially of an antigenic polypeptide sequence of the 259 Carboxy-terminal amino acids of V antigen. In particular embodiments, the immunogenic recombinant LcrV polypeptide is encoded by construct pAV13 shown in FIG. 1. In still other embodiments, the 259 carboxy-terminal amino acids of immunogenic V antigen LcrV polypeptide have the sequence of amino acid residues 68 to 326 of SEQ ID NO:1. In still further embodiments, the immunogenic LcrV polypeptide having an antigenic polypeptide sequence of the 259 carboxy-terminal amino acids of V antigen includes at least a 31.5 kDa portion of the 259 carboxy-terminal amino acids of V antigen. In yet another embodiment, the immunogenic LcrV polypeptide having an antigenic polypeptide sequence of the 259 carboxy-terminal amino acids of V antigen includes at least a 19.3 kDa portion of the 259 carboxy-terminal amino acids of V antigen. In still further embodiments, the immunogenic LcrV polypeptide having an antigenic polypeptide sequence of the 259 carboxy-terminal amino acids of V antigen includes at least a 31.5 kDa amino-terminal portion of the 259 carboxy-terminal amino acids of V antigen. In still further embodiments, the immunogenic LcrV polypeptide having an antigenic polypeptide sequence of the 259 carboxy-terminal amino acids of V antigen includes an antigenic polypeptide sequence of the 31.5 kDa amino-terminal portion of the 259 carboxy-terminal amino acids of V antigen. In yet other embodiments, the immunogenic LcrV polypeptide having an antigenic polypeptide sequence of the 259 carboxy-terminal amino acids of V antigen does not include an antigenic polypeptide of the 19.5 kDa amino-terminal portion of the 259 carboxy-terminal amino acids of V antigen. In yet other embodiments, the immnunogenic LcrV polypeptide having an antigenic polypeptide sequence of the 259 carboxy-terminal amino acids of V antigen does not include an antigenic polypeptide of the 19.5 kDa amino-terminal portion of the 259 carboxy-terminal amino acids of V antigen. In further embodiments, the immunogenic LcrV polypeptide having an antigenic polypeptide sequence of the 259 carboxy-terminal amino acids of V antigen does not a portion of the 19.5 kDa amino-terminal portion of the 259 carboxy-terminal amino acids of V antigen. In still further embodiments, the immunogenic LcrV polypeptide having an antigenic polypeptide sequence of the 259 carboxy-terminal amino acids of V antigen, the antigenic polypeptide sequence includes a sequence between amino acids 168 and 275. In other embodiments, the immunogenic LcrV polypeptide having an antigenic polypeptide sequence of the 259 carboxy-terminal amino acids of V antigen, the antigenic polypeptide sequence includes a sequence between amino acids 68 and 275.

In another aspect, the invention provides V antigen-based Yersinia vaccines that include an LcrV polypeptide sequence that include an immunogenic polypeptide sequence of the 259 carboxy-terminal amino acids of V antigen. In a further aspect, the invention provides V antigen-based Yersinia vaccines that consist essentially of an immunogenic polypeptide sequence of the 259 carboxy-terminal amino acids of V antigen LcrV polypeptide sequence. In particular embodiments, the V antigen-based Yersinia vaccine the immunogenic polypeptide subsequence is encoded by construct pAV13 shown in FIG. 1. In other embodiments, the V antigen-based Yersinia vaccine includes an immunogenic polypeptide subsequence that includes at least 31.5 kDa of the 259 carboxy-terminal amino acids of V antigen. In further embodiments, the V antigen-based Yersinia vaccine includes an immunogenic polypeptide subsequence that includes an antigenic polypeptide sequence of the 31.5 kDa amino-terminal portion of the 259 carboxy-terminal amino acids of V antigen. In still further embodiments, the V antigen-based Yersinia vaccine includes as immunogenic polypeptide subsequence that includes at least 19.3 kDa of the 259 Carboxy-terminal amino acids of V antigen. In yet further embodiments, the V antigen-based Yersinia vaccine includes an immunogenic polypeptide sequence that includes a sequence between amino acids 68 and 275. In other embodiments, the V antigen-based Yersinia vaccine includes an immunogenic polypeptide subsequence that is not a part of the 19.5 kDa amino-terminal portion of the 259 Carboxy-terminal amino acids of V antigen.

In another aspect, the invention provides an isolated antibody, or binding fragment thereof, that specifically binds to an antigenic polypeptide sequence of the 259 Carboxy-terminal amino acids of V antigen. In still other aspects, the invention provides an isolated antibody, or binding fragment thereof, that specifically binds to an antigenic polypeptide sequence of the 31.5 kDa amino-terminal portion of the 259 carboxy-terminal amino acids of V antigen. In particular embodiments, the isolated antibody, or binding fragment thereof, binds specifically to an antigenic polypeptide sequence that is not a part of the 19.5 kDa N-terminal portion of the 259 Carboxy-terminal amino acids of V antigen.

In yet another aspect, the invention provides polyclonal antisera that includes antibodies that specifically binds to antigenic polypeptide sequences of the 259 carboxy-terminal amino acids of V antigen. In another aspect, the invention provides polyclonal antisera that consists essentially of antibodies that specifically binds to antigenic polypeptide sequences of the 259 carboxy-terminal amino acids of V antigen, and do not bind to the amino-terminal 67 amino acids of V antigen. In still a further aspect, the invention provides polyclonal antisera that include antibodies that specifically bind to antigenic polypeptide sequences of the 31.5 kDa N-terminal portion of the 259 carboxy-terminal amino acids of V antigen. In particular aspects, the polyclonal antisera consist essentially of antibodies that specifically bind to antigenic polypeptide sequences of the 31.5 kDa N-terminal portion of the 259 carboxy-terminal amino acids of V antigen, and does not include antibodies that specifically bind to V antigen outside this region. In particular embodiments, the polyclonal antiserum includes antibodies that specifically bind to an antigenic polypeptide sequence that is not a part of the 19.5 kDa N-terminal portion of the 259 carboxy-terminal amino acids of V antigen.

In yet another aspect, the invention provides anti-Yersinia antisera made by injecting a mammal with an immunogenic amount of any of the above-described immunogenic LcrV polypeptides of the invention. In a further aspect, the invention provides an anti-bubonic plague antiserum made by injecting a mammal with an immunogenic amount of any of the above-described immunogenic LcrV polypeptides of the invention. In a further aspect, the invention provides methods of treating or preventing a Yersinia infection in a mammal by administering an immunoprotective amount of an anti-plague antiserum raised against any of the above-described immunogenic LcrV polypeptides of the invention. In still another aspect, the invention provides a method of treating or preventing bubonic plague in a mammal by administering an immunoprotective amount of an anti-plague antiserum raised against any of the above-described immunogenic LcrV polypeptides of the invention.

In still another aspect, the invention provides isolated recombinant V protein antigens truncated at their amino-terminal end by 67 amino acids and encoded by all but the 201 amino-terminal base pairs of a V antigen gene. In particular embodiments, the truncated recombinant V protein antigen is encoded by construct pAV13 shown in FIG. 1.

In yet another aspect, the invention provides a method of preventing or controlling Y. pestis in a mammal by providing a vaccine formulation that includes an isolated recombinant V protein antigen truncated at its amino-terminal end by 67 amino acids; and administering an effective immunizing amount of the vaccine to the mammal. In a further aspect, the invention provides a method of preventing or controlling Y. pestis in a mammal by providing a truncated recombinant V protein antigen encoded by construct pAV13 shown in FIG. 1 and administering an effective immunizing amount of the vaccine to the mammal. In still another aspect, the invention provides a method of treating a mammal infected with Y. pestis by providing a vaccine formulation that includes an isolated recombinant V protein antigen truncated at its amino-terminal end by 67 amino acids; and administering an effective immunizing amount of the vaccine to the mammal. In yet another aspect, the invention provides a method of treating a mammal infected with Y. pestis by providing a truncated recombinant V protein antigen encoded by construct pAV13 shown in FIG. 1 and administering an effective immunizing amount of the vaccine to the mammal.

In a further aspect, the invention provides a polypeptide consisting essentially of a Yersinia V-antigen immunogenic polypeptide having the sequence VLEELVQLVK DKNIDISIKY. In still further aspects, the invention provides a polypeptide consisting essentially of a Yersinia V-antigen immunogenic polypeptide having the sequence INLMDKNLYG YTDEEIFKAS. In yet another aspect, the immunogenic polypeptide mixture comprising a polypeptide consisting essentially of the sequence VLEELVQLVK DKNIDISIKY and a polypeptide consisting essentially of the sequence INLMDKNLYG YTDEEIFKAS. In still another embodiment, the amino and carboxy termini of the polypeptide consisting essentially of a Yersinia V-antigen immunogenic polypeptide having the sequence VLEELVQLVK DKNIDISIKY are joined intramolecularly to form cyclo[VLEELVQLVK DKNIDISIKY]. In yet other embodiments, the amino and carboxy termini of the polypeptide consisting essentially of a Yersinia V-antigen immunogenic polypeptide having the sequence INLMDKNLYG YTDEEIFKAS are joined intramolecularly to form cyclo[INLMDKNLYG YTDEEIFKAS].

In a further aspect, the invention provides a polypeptide having 2 or more contiguous repeats of the polypeptide sequence VLEELVQLVK DKNIDISIKY. In additional embodiments, the polypeptide has the form [VLEELVQLVK DKNIDISIKY]ₙ, where n is from two to five contiguous repeats of the sequence VLEELVQLVK DKNIDISIKY. In particular embodiments, the amino and carboxy termini are joined intramolecularly to form cyclo[VLEELVQLVK DKNIDISIKY]ₙ.

In still a further aspect, the invention provides a polypeptide having 2 or more contiguous repeats of the polypeptide sequence INLMDKNLYG YTDEEIFKAS. In additional embodiments, the polypeptide has the form [INLMDKNLYG YTDEEIFKAS]ₙ, where n is from two to five contiguous repeats of the sequence INLMDKNLYG YTDEEIFKAS. In particular embodiments, the amino and carboxy termini are joined intramolecularly to form cyclo[INLMDKNLYG YTDEEIFKAS]ₙ.

In another aspect, the invention provides immunogenic polypeptide conjugates having a Yersinia V-antigen polypeptide that consists essentially of a [VLEELVQLVK DKNIDISIKY]ₙ polypeptide, where n is one to five, linked to a carrier. In particular embodiments, the carrier is polylysine or polyserine.

In a further aspect, the invention provides immunogenic polypeptide conjugates having a Yersinia V-antigen polypeptide that consists essentially of a [INLMDKNLYG YTDEEWFKAS]ₙ polypeptide, wherein n is one to five, linked to a carrier. In particular embodiments, the carrier is polylysine or polyserine.

In another aspect, the invention provides a Yersinia V-antigen immunogenic polypeptide that includes one or more polypeptide repeats consisting essentially of the sequence VLEELVQLVK DKNIDISIKY and one or more polypeptide repeats consisting essentially of the sequence INLMDKNLYG YTDEEIFKAS. In particular embodiments, the Yersinia V-antigen immunogenic polypeptide has the sequence VLEELVQLVK DKNIDISIKY INLMDKNLYG YTDEEIFKAS. In further embodiments, the Yersinia V-antigen immunogenic polypeptide has the sequence INLMDKNLYG YTDEEIFKAS VLEELVQLVK DKNIDISIKY. In still further embodiments, the Yersinia V-antigen immunogenic polypeptide has the sequence VLEELVQLVK DKNIDISIKY INLMDKNLYG YTDEEIFKAS VLEELVQLVK DKNIDISIKY INLMDKNLYG YTDEEIFKAS. In yet further embodiments, the Yersinia V-antigen immunogenic polypeptide has the sequence INLMDKNLYG YTDEEIFKAS VLEELVQLVK DKNIDISIKY INLMDKNLYG YTDEEIFKAS. In still other embodiments, the amino and carboxy termini of the Yersinia V-antigen immunogenic polypeptide are joined intramolecularly to form a cyclic peptide. In particular embodiments, the amino and carboxy termini are joined intramolecularly to form cyclo[VLEELVQLVK DKNIDISIKY INLMDKNLYG YTDEEIFKAS]. In other particular embodiments, the amino and carboxy termini are joined intramolecularly to form cyclo[INLMDKNLYG YTDEEIFKAS VLEELVQLVK DKNIDISIKY]. In still further embodiments, the Yersinia V-antigen immunogenic polypeptide further includes a carrier, which can be polylysine or polyserine.

In still another aspect, the invention provides a Yersinia vaccine that includes any of the above-described Yersinia V-antigen immunogenic polypeptides or immunogenic polypeptide mixtures. In particular embodiments, the vaccine having any of the above-described Yersinia V-antigen immunogenic polypeptides or immunogenic polypeptide mixtures, further includes a protein carrier. In other embodiments, the vaccine having any of the above-described Yersinia V-antigen immunogenic polypeptides or immunogenic polypeptide mixtures, further includes an adjuvant. In particular embodiments, the adjuvant can be alum, a polymer adjuvant, a co-polymer adjuvant, Freund's complete adjuvant, Freund's incomplete adjuvant, sorbitan monooleate, QS 21, muramyl dipeptide, a CpG oligonucleotide adjuvant, trehalose, a bacterial extract adjuvant, a detoxified endotoxin adjuvant, a membrane lipid adjuvant, or a combination of any of these adjuvant agents. In particularly useful embodiments, the vaccine further includes an immunogenic Pla polypeptide sequence, such as a polypeptide of the sequence shown in FIG. 19A. In further particular embodiments the Pla polypeptide consists essentially of the Pla polypeptide sequence [ATGGSYSYNNGAYTGNFPKGVRVIGYNQRF]ₙ, where n is 1 or a multiple of contiguous repeats. In other particular embodiments, the immunogenic Pla polypeptide consists essentially of [RAHDNDEHYMRDLTFREKTS]ₙ, where n is 1 or a multiple of contiguous repeats. In still other embodiments, the immunogenic Pla polypeptide consists essentially of [KGGTQTIDKNSGDSVSIGGDAAGISNKN]ₙ, where n is 1 or a multiple of contiguous repeats. In still further embodiments, the Pla polypeptide consists essentially of the polypeptide of SEQ ID No. 9. In yet other embodiments, the vaccine further includes a Psn polypeptide sequence, such as a polypeptide of the sequence shown in FIG. 20A. In particular embodiments, the Psn polypeptide consists essentially of the polypeptide of SEQ ID No. 11.

In another aspect, the invention provides a Yersinia vaccine that includes an immunogenic Pla polypeptide. In particular embodiments, the immunogenic Pla polypeptide consists essentially of [ATGGSYSYNNGAYTGNFPKGVRVIGYNQRF]ₙ, where n is 1 or a multiple of contiguous repeats. In other embodiments, the immunogenic Pla polypeptide consists essentially of [RAHDNDEHYMRDLTFREKTS]ₙ, wherein n is 1 or a multiple of contiguous repeats. In still other embodiments, the immunogenic Pla polypeptide consists essentially of [KGGTQTIDKNSGDSVSIGGDAAGISNKN]ₙ, where n is 1 or a multiple of contiguous repeats. In further embodiments, the immunogenic Pla polypeptide consists essentially of the polypeptide of SEQ ID No. 9. In another embodiment, the vaccine further includes a Psn polypeptide. In particular embodiments, the Psn polypeptide consists essentially of the polypeptide of SEQ ID No. 11. In other embodiments, the vaccine further includes a protein carrier. In further particularly useful embodiments, the vaccine includes an adjuvant. In particular embodiments, the adjuvant can be alum, a polymer adjuvant, a co-polymer adjuvant, Freund's complete adjuvant, Freund's incomplete adjuvant, sorbitan monooleate, QS 21, muramyl dipeptide, a CpG oligonucleotide adjuvant, trehalose, a bacterial extract adjuvant, a detoxified endotoxin adjuvant, a membrane lipid adjuvant, or a combination of any of these adjuvant agents. In still further embodiments, the Yersinia vaccine that includes an immunogenic Pla polypeptide also includes a V-antigen polypeptide.

In another aspect, the invention provides Yersinia vaccines that include an immunogenic Pla polypeptide and any of the above-described immunogenic Yersinia V-antigen polypeptides or polypeptide mixtures of the invention.

In a further aspect, the invention provides methods of treating or preventing a Yersinia infection in a mammal by administering to the mammal an immunogenic amount of a polypeptide of any of the above-described immunogenic Yersinia V-antigen polypeptides or polypeptide mixtures of the invention. In particular embodiments, the mammal treated is a human. In another aspect, the invention provides methods of treating or preventing a Yersinia infection in a mammal by administering to the mammal an immunogenic amount of a Yersinia vaccine that includes any of the above-described Yersinia V-antigen immunogenic polypeptides or immunogenic polypeptide mixtures. In particular embodiments, the mammal treated is a human. In a further aspect, the invention provides methods of treating or preventing a Yersinia infection in a mammal by administering to the mammal an immunogenic amount of a Yersinia vaccine that includes an immunogenic Pla polypeptide sequence, such as a sequence of the Pla polypeptide shown in FIG. 19A. In particular embodiments, the mammal treated is a human. In still another aspect, the invention provides methods of treating or preventing a Yersinia infection in a mammal by administering to the mammal an immunogenic amount of a Yersinia vaccine that include an immunogenic Pla polypeptide and any of the above-described immunogenic Yersinia V-antigen polypeptides or polypeptide mixtures of the invention. In particular embodiments, the mammal treated is a human.

In another aspect the invention provides a method of treating or preventing a Yersinia infection in a mammal by administering to the mammal an immunoprotective amount of antibodies from an immune serum of a second mammal that has been treated with an immunogenic amount of a polypeptide of any of the above-described immunogenic Yersinia V-antigen polypeptides or polypeptide mixtures of the invention. In particular embodiments, the mammal treated is a human. In another aspect the invention provides a method of treating or preventing a Yersinia infection in a mammal by administering to the mammal an immunoprotective amount of antibodies from an immune serum of a second mammal that has been treated with an immunogenic amount of a Yersinia vaccine that includes any of the above-described Yersinia V-antigen immunogenic polypeptides or immunogenic polypeptide mixture. In particular embodiments, the mammal treated is a human. In another aspect the invention provides a method of treating or preventing a Yersinia infection in a mammal by administering to the mammal an immunoprotective amount of antibodies from an immune serum of a second mammal that has been treated with an immunogenic amount of a Yersinia that includes an immunogenic Pla polypeptide sequence, such as a sequence of the Pla polypeptide shown in FIG. 19A. In particular embodiments, the mammal treated is a human.

In another important aspect, the invention provides polypeptides that include an immunogenic Yersinia V-antigen consensus polypeptide consisting essentially of VLEELXXXXX DKN. In further important aspects, the invention provides a polypeptide having an immunogenic Yersinia V-antigen consensus polypeptide consisting essentially of DKNXXX XTDEEIF. In still other important aspects, the invention provides an immunogenic polypeptide mixture having at least one polypeptide that carries the immunogenic Yersinia V-antigen consensus sequence VLEELXXXXX DKN; and at least one other polypeptide that carries the immunogenic Yersinia V-antigen consensus sequence DKNXXX XTDEEIF. In still another important aspect, the invention provides an immunogenic polypeptide conjugate that includes include a Yersinia V-antigen consensus polypeptide consisting essentially of VLEELXXXXX DKN or DKNXXX XTDEEIF linked to a carrier.

In a further particularly useful aspect, the invention provides a Yersinia vaccine having any of the above-described immunogenic polypeptides or immunogenic polypeptide mixtures of the invention. In particular embodiments, the vaccine further includes a protein carrier. In particularly useful embodiments, the vaccine further includes an adjuvant. In particular embodiments, the adjuvant is alum, a polymer adjuvant, a co-polymer adjuvant, Freund's complete adjuvant, Freund's incomplete adjuvant, sorbitan monooleate, QS 21, muramyl dipeptide, a CpG oligonucleotide adjuvant, trehalose, a bacterial extract adjuvant, a detoxified endotoxin adjuvant, a membrane lipid adjuvant, or any combination of these adjuvant agents.

In still further aspects, the invention provides methods of treating or preventing a Yersinia infection in a mammal by administering to the mammal an immunogenic amount of a polypeptide of any of the above-described immunogenic polypeptides or immunogenic polypeptide mixtures of the invention. In particular embodiments, the mammal treated is a human. In another aspect, the invention provides a method of treating or preventing a Yersinia infection in a mammal by administering to the mammal an immunogenic amount of a Yersinia vaccine having any of the above-described immunogenic polypeptides or immunogenic polypeptide mixtures of the invention. In particular embodiments, the Yersinia vaccine administered includes an adjuvant, such as alum, a polymer adjuvant, a co-polymer adjuvant, Freund's complete adjuvant, Freund's incomplete adjuvant, sorbitan monooleate, QS 21, muramyl dipeptide, a CpG oligonucleotide adjuvant, trehalose, a bacterial extract adjuvant, a detoxified endotoxin adjuvant, a membrane lipid adjuvant, or any combination of these agents. In particular embodiments, the mammal treated is a human.

In a further important aspect, the invention provides a method of treating a first mammal, e.g., a human, by first administering to a second mammal an immunogenic amount of a polypeptide of any of the above-described immunogenic polypeptides or immunogenic polypeptide mixtures of the invention, and then collecting immune serum from the second mammal and administering the immune serum to the first mammal, e.g. a human in need thereof. Accordingly, the invention provides methods of treating or preventing a Yersinia infection in a mammal, e.g., a human, by administering to the mammal an immunoprotective amount of antibodies from an immune serum of a second mammal that has been treated with an immunogenic amount of a polypeptide of any of the above-described immunogenic polypeptides or immunogenic polypeptide mixtures of the invention.

In another important aspect, the invention provides a method of screening for a Yersinia infection immunomodulatory compound by first contacting a V-antigen binding unit (e.g., LcrV, or a polypeptide having a Yersinia V-antigen consensus polypeptide consisting essentially of VLEELXXXXX DKN or DKNXXX XTDEEIF) with an interferon gamma receptor/interferon gamma ligand ternary complex (IFN-γR-IFN-γ) in the presence of a test compound, and then measuring the amount of binding of the V-antigen binding unit to the interferon gamma receptor/interferon gamma ligand ternary complex (IFN-γR-IFN-γ) in the presence of the test compound. The amount of binding of the V-antigen binding unit to the interferon gamma receptor/interferon gamma ligand ternary complex (IFN-γR-IFN-γ) in the presence of the test compound with the amount of binding of the V-antigen binding unit to the interferon gamma receptor/interferon gamma ligand ternary complex (IFN-γR-IFN-γ) in the absence of the test compound. In general, the test compound is a Yersinia infection immunomodulatory compound if the amount of binding in the presence of the test compound is different, particularly lower, than the amount of binding in the absence of the test compound. In particular embodiments, the interferon gamma receptor/interferon gamma ligand ternary complex (IFN-γR-IFN-γ) is expressed on the surface of a CD14-negative cell, such as a human monocyte or a human neutrophilic leukocyte.

In yet another aspect, the invention provides a method of stimulating an anti-inflammatory response in a host by administering an LcrV protein or polypeptide. In particular embodiment, the anti-inflammatory response stimulated includes upregulation of an anti-inflammatory cytokine. In further particular embodiments, the anti-inflammatory cytokine that is upregulated is IL-10.

In a further aspect, the invention provides a method of stimulating an anti-inflammatory response in a host by administering an LcrV protein or polypeptide having a TLR-2 receptor binding site that includes amino acid residues 32 to 35 or 203 to 206 of SEQ ID NO: 1. In particular embodiments, the LcrV protein or polypeptide includes the consensus sequence VLEELXXXXX DKN. In other embodiments, the LcrV protein or polypeptide consists essentially of the consensus sequence VLEELXXXXX DKN. In a further embodiment, the LcrV protein or polypeptide includes the sequence VLEELVQLVK DKNIDISIDY. In yet other embodiments, the LcrV protein or polypeptide includes the consensus sequence DKNXXX XTDEEIF. In other embodiments, the LcrV protein or polypeptide consists essentially of the consensus sequence DKNXXX XTDEEIF. In particular embodiments, the LcrV protein or polypeptide includes the sequence VLEELVQLVK DKNIDISIDY. In further embodiments of this method of the invention, the host has an inflammatory disease or disorder. In other embodiments, the host has cancer. In still other embodiments, the host is infected with HIV. In yet other embodiments, the host has an allograft.

In another aspect, the invention provides a method of treating or preventing an inflammatory disease or condition in a host by administering a pharmaceutically effective amount of an LcrV protein or polypeptide. In particular embodiments, the LcrV protein or polypeptide has a TLR-2 receptor binding site that includes amino acid residues 32 to 35 or 203 to 206 of SEQ ID NO: 1. In particular embodiments, the LcrV protein or polypeptide includes the consensus sequence VLEELXXXXX DKN. In other embodiments, the LcrV protein or polypeptide consists essentially of the consensus sequence VLEELXXXXX DKN. In a further embodiment, the LcrV protein or polypeptide includes the sequence VLEELVQLVK DKNIDISIDY. In yet other embodiments, the LcrV protein or polypeptide includes the consensus sequence DKNXXX XTDEEIF. In other embodiments, the LcrV protein or polypeptide consists essentially of the consensus sequence DKNXXX XTDEEIF. In particular embodiments, the LcrV protein or polypeptide includes the sequence VLEELVQLVK DKNIDISIDY. In further embodiments of this method of the invention, the inflammatory condition is an allograft. In other embodiments, the inflammatory condition is a wound in need of healing. In still other embodiments, the LcrV protein or polypeptide used represses inflammation but not specific immunity. In particular embodiments, the inflammatory disease or condition is systemic lupus (erythematosus), multiple sclerosis, inflammatory bowel disease, rheumatoid arthritis, septic shock, erythema nodosum leprosy, septicemia, or uveitis.

In a further aspect, the invention provides a method of inhibiting an NF-κB-dependent disease process in a host by administering a pharmaceutically effective amount of an LcrV protein or polypeptide. In particular embodiments, the NF-κB-dependent disease process is a malignant cell growth. In other embodiment, the NF-κB-dependent disease process is HIV replication. In further particular embodiments, the LcrV protein or polypeptide has a TLR-2 receptor binding site that includes amino acid residues 32 to 35 or 203 to 206 of SEQ ID NO: 1. In particular embodiments, the LcrV protein or polypeptide includes the consensus sequence VLEELXXXXX DKN. In other embodiments, the LcrV protein or polypeptide consists essentially of the consensus sequence VLEELXXXXX DKN. In a further embodiment, the LcrV protein or polypeptide includes the sequence VLEELVQLVK DKNIDISIDY. In yet other embodiments, the LcrV protein or polypeptide includes the consensus sequence DKNXXX XTDEEIF. In other embodiments, the LcrV protein or polypeptide consists essentially of the consensus sequence DKNXXX XTDEEIF. In particular embodiments, the LcrV protein or polypeptide includes the sequence VLEELVQLVK DKNIDISIDY.

### 3. BRIEF DESCRIPTION OF THE FIGURES

FIG. 1A is a schematic representation of the construction of recombinant plasmid pPA V13 encoding a staphylococcal protein A-V antigen fusion peptide termed PAV.

FIG. 1B is a schematic representation of the characterization of PAV with the sites of acid-labile Asp-Pro cleavage sites indicated by arrowheads.

FIG. 2 is a diagrammatic representation of the deletional variants of V antigen constructed from the pBVP5 clone of lcrGVH-yopBD operon of the Lcr plasmid of Y. pseudotuberculosis.

FIG. 3A is a photographic representation of a protein gel loaded with extracts of E. coli containing vector plasmid pK223-3 (lane 1) or recombinant plasmid PKVE14 (lane 2), or various stages of purified V antigen prepared from the recombinant plasmid PKVE14 E. coli extract (lanes 3-8).

FIG. 3B is a photographic representation of an immunoblot of the protein gel in FIG. 3A immunoblotted against rabbit polyclonal anti-native V antigen.

FIG. 3C is a photographic representation of an immunoblot of the protein gel in FIG. 3A immunoblotted against mouse monoclonal anti-native V antigen 5A4.8.

FIG. 3D is a photographic representation of an immunoblot of the protein gel in FIG. 3A immunoblotted against mouse monoclonal anti-native V antigen 3A4.1.

FIG. 3E is a photographic representation of an immunoblot of the protein gel in FIG. 3A immunoblotted against rabbit polyclonal anti-PAV.

FIG. 3F is a photographic representation of an immunoblot of the protein gel in FIG. 3A immunoblotted against rabbit polyclonal anti-truncated staphylococcal protein A.

FIG. 4A is a photographic representation of an immunoblot of various V antigen preparations prepared with polyclonal anti-native V antigen.

FIG. 4B is a photographic representation of an immunoblot of various V antigen preparations prepared with mouse monoclonal anti-V antigen 17A5.1 directed against truncated protein A.

FIG. 5A is a photographic representation of an immunoblot of extracts of Ca²⁺-starved whole cells of various Yersinia bacterial species, and strains thereof, prepared with absorbed rabbit polyclonal anti-native V antigen purified from Y. pestis KIM.

FIG. 5B is a photographic representation of an immunoblot of extracts of Ca²⁺-starved whole cells of various Yersinia bacterial species, and strains thereof, prepared with anti-recombinant V antigen.

FIG. 5C is a photographic representation of an immunoblot of extracts of Ca²⁺-starved whole cells of various Yersinia bacterial species, and strains thereof, prepared with prepared with anti-PAV.

FIG. 5D is a photographic representation of an immunoblot of extracts of Ca²⁺-starved whole cells of various Yersinia bacterial species, and strains thereof, prepared with anti-truncated protein A.

FIG. 6A is a photographic representation of an immunoblot prepared with absorbed rabbit polyclonal anti-native V antigen blotted against bacterial extracts from control (lane 1) and various V-antigen recombinant E. coli hosts (lanes 2-6) as well as Lcr⁺ Y. pestis KIM (lane 7), and Lcr⁻ Y. pestis KIM (lane 8).

FIG. 6B is a photographic representation of an immunoblot prepared with mouse monoclonal anti-V antigen 15A4.8 blotted against bacterial extracts from control (lane 1) and various V-antigen recombinant E. coli hosts (lanes 2-6) as well as Lcr⁺ Y. pestis KIM (lane 7), and Lcr⁻ Y. pestis KIM (lane 8).

FIG. 6C is a photographic representation of an immunoblot prepared with mouse monoclonal anti-V antigen 17A5.1 blotted against bacterial extracts from control (lane 1) and various V-antigen recombinant E. coli hosts (lanes 2-6) as well as Lcr⁺ Y. pestis KIM (lane 7), and Lcr⁻ Y. pestis KIM (lane 8).

FIG. 7A is a photographic representation of an immunoblot prepared with rabbit polyclonal anti-PAV (without absorption) blotted against bacterial extracts expressing V₀ (lane 1), V₁, (lane 2), V₂ (lane 3), and a vector plasmid control) (lane 4).

FIG. 7B is a photographic representation of an immunoblot prepared with rabbit polyclonal anti-PAV (after exhaustive absorption with preparations of E. coli BL21 (DE3) transformed with pBluescript SK⁺ containing shared proteins alone) blotted against bacterial extracts expressing V₀ (lane 1), V₁, (lane 2), V₂ (lane 3), and a vector plasmid control (lane 4).

FIG. 7C is a photographic representation of an immunoblot prepared with rabbit polyclonal anti-PAV (after exhaustive absorption with preparations of E. coli BL21[(DE3) transformed with pVBP514D shared proteins plus excess V₂) blotted against bacterial extracts expressing V₀ (lane 1), V₁, (lane 2), V₂ (lane 3), and a vector plasmid control (lane 4).

FIG. 7D is a photographic representation of an immunoblot prepared with rabbit polyclonal anti-PAV (after exhaustive absorption with preparations of E. coli BL21 (DE3) transformed with pBVP53D shared proteins plus excess V₁) blotted against bacterial extracts expressing V₀ (lane 1), V₁, (lane 2), V₂ (lane 3), and a vector plasmid control (lane 4).

FIG. 7E is a photographic representation of an immunoblot prepared with rabbit polyclonal anti-PAV (after exhaustive absorption with preparations of E. coli BL21(DE3) transformed with pBVP5 containing shared proteins alone shared proteins plus excess V₀) blotted against bacterial extracts expressing V₀ (lane 1), V₁, (lane 2), V₂ (lane 3), and a vector plasmid control (lane 4).

FIG. 8 is a diagrammatic representation summarizing the ability of IgG isolated from normal rabbit serum, as well as antisera raised against various V antigen antisera preparations indicated and a protein A control, to provide passive immunity against intravenous challenge with the various indicated Yersinia species and strains.

FIG. 9A is a schematic representation of the polypeptide sequence of a Y. pestis V antigen (SEQ ID NO. 1) showing the conserved dual binding sites in bold and conserved EE motif underlined (see also GenBank Accession No. CAB54908), and the 76 amino acids deleted from the N-terminal truncated derivative (LCRV₆₈₋₃₂₆) indicated in italics.

FIG. 9B is a schematic representation of the nucleotide sequence of a Y. pestis V antigen-encoding nucleic acid sequence corresponding to GenBank Accession No. AL117189 (SEQ ID NO. 2) showing the initiation and termination codons of the V-antigen coding sequence are underlined.

FIG. 10A is a graphical representation of a scatchard analysis of the specific binding of ¹²⁵I-LcrV to the synthetic fragment of the mouse TLR-2 extracellular domain.

FIG. 10B is a graphical representation of a Scatchard plot of the specific binding of ¹²⁵I-LcrV₆₈₋₃₂₆ to the synthetic fragment of the mouse TLR-2 extracellular domain.

FIG. 10C is a graphical representation of a Scatchard plot of the specific binding of ¹²⁵I-LcrV₁₉₃₋₂₁₀ to the synthetic fragment of the mouse TLR-2 extracellular domain.

FIG. 10D is a graphical representation of a Scatchard plot of the specific binding of ¹²⁵I-LcrV₃₁₋₅₀ to the synthetic fragment of the mouse TLR-2 extracellular domain.

FIG. 11A is a graphical representation of a Scatchard plot analysis of the specific binding of ¹²⁵I-LcrV₃₁₋₅₀ to human thymic epithelial VTEC2.HS cells.

FIG. 11B is a graphical representation of a Scatchard plot analysis of the specific binding of ¹²⁵I-LcrV₆₈₋₃₂₆ to human thymic epithelial VTEC2.HS cells.

FIG. 11C is a graphical representation of a competitive binding analysis illustrating inhibition of the specific binding of ¹²⁵I-LcrV₃₁₋₅₀ to VTEC2.HS cells by unlabeled LcrV (open circles) or LcrV₆₈₋₃₂₆ (filled circles).

FIG. 12A is a graphical representation of a Scatchard plot analysis of the specific binding of ¹²⁵I-LcrV to U937 human monocytic leukemia cells in the presence of IFN-γ.

FIG. 12B is a graphical representation of a Scatchard plot analysis of the specific binding of ¹²⁵I-LcrV₆₈₋₃₂₆ to U937 human monocytic leukemia cells in the presence of IFN-γ.

FIG. 12C is a graphical representation of a Scatchard plot analysis of the specific binding of ¹²⁵I-LcrV₃₁₋₅₀ to U937 human monocytic leukemia cells in the presence of IFN-γ.

FIG. 12D is a graphical representation of a Scatchard plot analysis of the specific binding of ¹²⁵I-LcrV to U937 human monocytic leukemia cells in the presence of the IFN-γC-terminal peptide SQMLFRGRRASQ.

FIG. 13 is a graphical representation of the expression of IL-10 in culture supernatants of human monocytes after addition of 120 nM of LcrV (filled triangles), LcrV₆₈₋₃₂₆ (filled inverted triangles), LcrV₃₁₋₅₀ (filled diamonds), and LPS (1.0 μg/ml) provided 1 hr. before treatment with LcrV (filled squares).

FIG. 14 is a graphical representation of an experiment showing the inhibition of LPS-induced expression of TNF-α in human monocytes by LcrV.

FIG. 15 is a graphical representation of an experiment showing the inhibition of the oxidative burst of neutrophils (open bars) in the presence of LcrV (120 nM) (closed bars).

FIG. 16A is a schematic representation of the polypeptide sequence of a Y. pseudotuberculosis V antigen (SEQ ID NO:3).

FIG. 16B is a schematic representation of the nucleotide sequence of a Y. pseudotuberculosis V antigen-encoding nucleic acid sequence (SEQ ID NO:4).

FIG. 17A is a schematic representation of the polypeptide sequence of a Y. pestis V antigen (SEQ ID NO:5).

FIG. 17B is a schematic representation of the nucleotide sequence of a Y. pestis V antigen-encoding nucleic acid sequence (SEQ ID NO:6).

FIG. 18A is a schematic representation of the polypeptide sequence of a Y. enterocolitica V antigen (SEQ ID NO:7).

FIG. 18B is a schematic representation of the nucleotide sequence of a Y. enterocolitica V antigen-encoding nucleic acid sequence (SEQ ID NO:8).

FIG. 19A is a schematic representation of the polypeptide sequence of a Y. pestis Pla antigen (SEQ ID NO:9).

FIG. 19B is a schematic representation of the nucleotide sequence of a Y. pestis Pla antigen-encoding nucleic acid sequence (SEQ ID NO:10).

FIG. 20A is a schematic representation of the polypeptide sequence of a Y. pestis Psn antigen (SEQ ID NO:11).

FIG. 20B is a schematic representation of the nucleotide sequence of a Y. pestis Psn antigen-encoding nucleic acid sequence (SEQ ID NO:12).

### 4. DETAILED DESCRIPTION OF THE INVENTION

All patents, patent applications and publications cited herein are hereby incorporated by reference in their entirety. The disclosures of these publications in their entireties are thereby included with this application in order to more fully describe the state of the art as known to those skilled therein as of the date of the invention described and claimed herein.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986). The scientific literature referred to herein establishes the knowledge that is available to those with skill in the art, and is formally incorporated by reference herein.

4.1 General

In general, the instant invention provides Yersinia pestis LcrV-related compositions and associated methods of use for preventing and/or treating Yersinia pestis infection. In particular, the invention provides LcrV protein deletions, truncations, polypeptide fragments and protein fusions, that are useful in raising immunoprotective antibodies and in providing immunoprotective vaccine compositions. The invention further provides immunomodulatory LcrV proteins and polypeptides that are related to conserved TLR2 and IFN-γR-IFN-γ-binding subregions of LcrV, as well as associated methods of use for preventing pro-inflammatory responses, facilitating allograft retention and treating certain infectious diseases, such as HIV, and cancers.

The following detailed description of the elements and exemplary embodiments of the invention are provided in support of the claimed invention summarized above.

4.2 Definitions

The terms “a”, “an” and “the” as used herein are defined to mean one or more and include the plural unless the context is inappropriate.

As used herein, the term “about” means approximately, in the region of, roughly, or around. When the term “about” is used in conjunction with a numerical range, it modifies that range by extending the boundaries above and below the numerical values set forth, e.g., to modify a numerical value by plus or minus 10% of the stated value, rounded to the nearest whole number.

The use of the term “or” in the claims is used to mean “and/or” unless explicitly indicated to refer to alternatives only or the alternatives are mutually exclusive, although the disclosure supports a definition that refers to only alternatives and “and/or.”

“Biological property” when used in conjunction with LcrV protein or polypeptides means having any of the activities associated with a native LcrV protein or polypeptide. Such biological activities include, but are not limited to, TLR-2 binding and repression of innate immunity and pro-inflammatory cytokines.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

The terms “Peptides”, “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. The terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free alpha-amino group on the amino acid at the amino terminal of the peptide, or to the alpha-amino group (imino group when participating in a peptide bond) of an amino acid at any other location within the peptide. Similarly, the term “carboxy terminus” (abbreviated C-terminus) refers to the free carboxyl group on the amino acid at the carboxy terminus of a peptide, or to the carboxyl group of an amino acid at any other location within the peptide.

Furthermore, one of skill in the art will recognize that individual substitutions, deletions or additions in the amino acid sequence of the proteins and polypeptides, or in the nucleotide sequence encoding for the amino acids in the proteins and polypeptides, which alter, add or delete a single amino acid or a small percentage of amino acids (typically less than 5%, more typically less than 1%) in an encoded sequence are conservatively modified variations, wherein the alterations result in the substitution of an amino acid with a chemically similar amino acid. Conservative substitution tables providing functionally similar amino acids are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

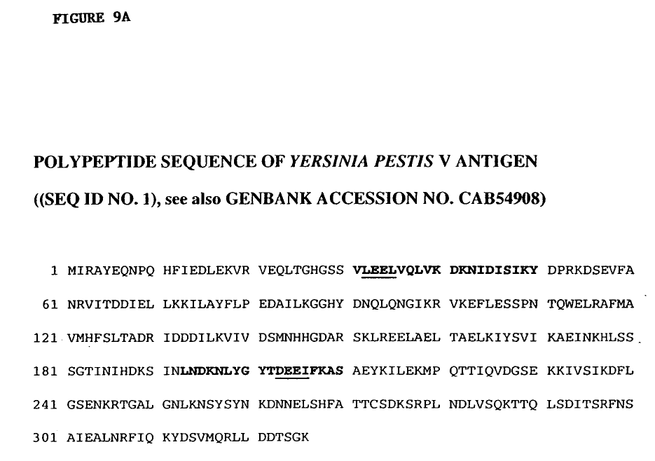

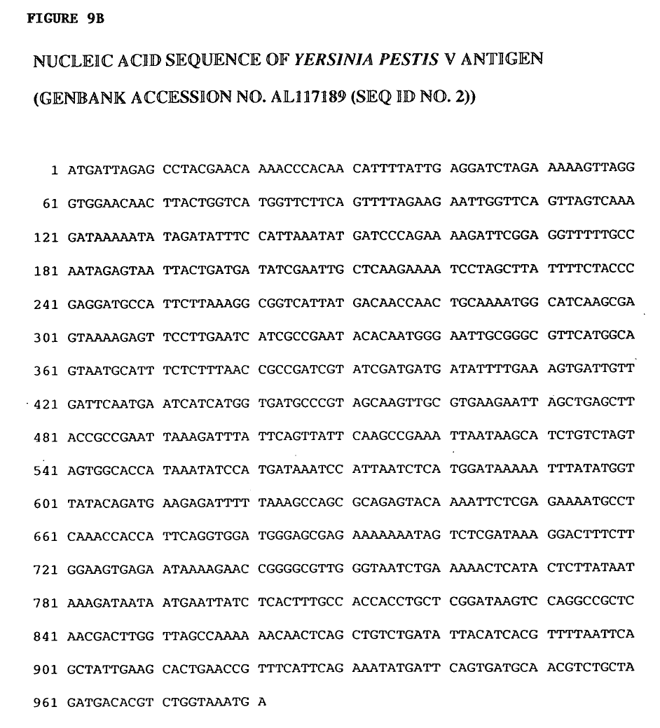

An “LcrV protein or polypeptide fragment” is a portion of a naturally occurring full-length LcrV protein or polypeptide sequences having one or more amino acid residues deleted. The deleted amino acid residue(s) may occur anywhere in the polypeptide, including at either the N-terminal or C-terminal end or internally. Accordingly, a “LcrV protein or polypeptide fragment” of the invention may or may not possess one or more biological activities of LcrV protein or polypeptide. LcrV protein or polypeptide fragments typically, will have a consecutive sequence of at least 20, 30, or 40 amino acid residues of a LcrV protein or polypeptide (e.g., Y. pestis LcrV protein or polypeptide alpha and beta subunits shown in FIG. 9A. Useful LcrV protein or polypeptide fragments have about 20-100 residues, which are identical to the sequence of human LcrV protein or polypeptide. Other LcrV protein or polypeptide fragments include those produced as a result of chemical or enzymatic hydrolysis or digestion of the purified LcrV protein or polypeptide, as described further herein.

The term “LcrV variants” or “LcrV sequence variants” as defined herein mean biologically active LcrV as defined below having less than 100% sequence identity with a native Yersinia LcrV polypeptide that is isolated from, e.g., the deduced amino acid sequence shown in FIG. 9A. Ordinarily, a biologically active LcrV protein or polypeptide variant has an amino acid sequence having at least about 70% amino acid sequence identity with human LcrV protein or polypeptide, at least about 75%, or at least about 80%, or at least about 85%, or at least about 90%, and at least about 95%, at least about 97%, at least about 98%, at least about 99% or higher.

“Percent amino acid sequence identity” with respect to the LcrV protein or polypeptide sequence is defined herein as the percentage of amino acid residues in the candidate sequence that are identical with the residues in a LcrV protein or polypeptide polypeptide sequence, e.g., a LcrV protein or polypeptide alpha and beta subunits shown in FIG. 9A, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the LcrV protein or polypeptide sequence is construed as affecting sequence identity or homology. Percent amino acid sequence identity may be conveniently determined using an appropriate algorithm (e.g., the BLAST algorithm available through NCBI at www.ncbi.nlm.nih.gov/).

A “chimeric LcrV protein or polypeptide” is a polypeptide comprising full-length LcrV protein or polypeptide or one or more fragments thereof fused or bonded to a second protein or one or more fragments thereof.

The term “epitope tagged,” when used herein, refers to a chimeric polypeptide comprising an entire LcrV protein or polypeptide sequence, or a portion thereof, fused to a “tag polypeptide”. The tag polypeptide has enough residues to provide an epitope against which an antibody there against can be made, yet is short enough such that it does not interfere with activity of the LcrV protein or polypeptide. The tag polypeptide may be fairly unique so that the antibody there against does not substantially cross-react with other epitopes. Suitable tag polypeptides generally have at least 6 amino acid residues and usually between about 8-50 amino acid residues (typically between about 9-30 residues).

“Isolated LcrV protein or polypeptide”, “highly purified LcrV protein or polypeptide” and “substantially homogeneous LcrV protein or polypeptide” are used interchangeably and mean LcrV protein or polypeptide that has been purified from a LcrV protein or polypeptide source or has been prepared by recombinant or synthetic methods and is sufficiently free of other peptides or proteins. “Homogeneous” here means less than about 10 or less than about 5% contamination with other source proteins.

An “antigenic function” means possession of an epitope or antigenic site that is capable of cross-reacting with antibodies raised against native sequence LcrV protein or polypeptide. The principal antigenic function of a LcrV protein or polypeptide is that it binds with an affinity of at least about 10⁶ L/mole (binding affinity constant, i.e., Kₐ) to an antibody raised against LcrV protein or polypeptide. Ordinarily the polypeptide binds with an affinity of at least about 10⁷ L/mole. The binding affinity of the subject LcrV protein or polypeptide antibodies may also be measured in terms of a binding dissociation constant (K_{d}), which refers to the concentration of a binding protein (i.e., the antibody) at which 50% of the antigen protein (i.e., LcrV protein or polypeptide) is occupied. In general, particularly useful LcrV protein or polypeptide antibodies of the invention have a K_{d} value in the range of 0.1 to 3 nM (corresponding to a Kₐ of approximately 3×10⁸ L/mole to 1×10¹⁰ L/mole).

“Antigenically active” LcrV protein or polypeptide is defined as a polypeptide that possesses an antigenic function of LcrV protein or polypeptide, and that may (but need not) in addition possess a biological activity of LcrV protein or polypeptide.

The word “sample” refers to body fluid, tissue or a cell from a patient. Normally, the body fluid, tissue or cell will be removed from the patient, but in vivo diagnosis is also contemplated. A particularly useful sample is whole blood or blood serum. Other patient samples, including urine, serum, sputum, cell extracts, lymph, spinal fluid, feces and the like, are also included within the meaning of the term.

“Isolated LcrV protein or polypeptide nucleic acid” is RNA or DNA containing greater than 16, and typically 20 or more, sequential nucleotide bases that encodes biologically active LcrV protein or polypeptide or a fragment thereof, is complementary to the RNA or DNA, or hybridizes to the RNA or DNA and remains stably bound under moderate to stringent conditions. This RNA or DNA is free from at least one contaminating source nucleic acid with which it is normally associated in the natural source and typically substantially free of any other mammalian RNA or DNA. The phrase “free from at least one contaminating source nucleic acid with which it is normally associated” includes the case where the nucleic acid is present in the source or natural cell but is in a different chromosomal location or is otherwise flanked by nucleic acid sequences not normally found in the source cell. An example of isolated LcrV protein or polypeptide nucleic acid is RNA or DNA that encodes a biologically active LcrV protein or polypeptide sharing at least 75%, 80%, 85%, 90%, or even 95% sequence identity with the human LcrV protein or polypeptide alpha and beta subunits shown in FIG. 9A.

The expression “labeled” when used herein refers to a molecule (e.g., LcrV protein or polypeptide or anti-LcrV protein or polypeptide antibody) that has been conjugated, directly or indirectly, with a detectable compound or composition. The label may be detectable by itself (e.g., radioisotope labels or fluorescent labels) or, in the case of an enzymatic label, may catalyze a chemical alteration of a substrate compound or composition, which is detectable. A particularly useful label is an enzymatic one which catalyzes a color change of a non-radioactive color reagent.

“Operably linked” when referring to nucleic acids means that the nucleic acids are placed in a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The term “antibody” is used in the broadest sense and specifically covers single anti-LcrV protein or polypeptide monoclonal antibodies and anti-LcrV antibody compositions with polyepitopic specificity (including neutralizing and non-neutralizing antibodies).

The term “monoclonal antibody” as used herein refers to an antibody obtained from a population of substantially homogeneous antibodies, i.e., the individual antibodies comprising the population are identical except for possible naturally occurring mutations that may be present in minor-amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. Novel monoclonal antibodies or fragments thereof mean in principle all immunoglobulin classes such as IgM, IgG, IgD, IgE, IgA or their subclasses such as the IgG subclasses or mixtures thereof. IgG and its subclasses are particularly useful, such as IgG₁, IgG₂, IgG₂ₐ, IgG2b, IgG₃ or IgGM. The IgG subtypes IgG_{1/kappa} and IgG_{2b/lkapp} are included.

The monoclonal antibodies herein include hybrid and recombinant antibodies produced by splicing a variable (including hypervariable) domain of an anti-LcrV protein or polypeptide antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)2, and Fv), so long as they exhibit the desired biological activity. (See, e.g., U.S. Pat. No. 4,816,567 and Mage & Lamoyi, in Monoclonal Antibody Production Techniques and Applications, pp. 79-97 (Marcel Dekker, Inc.), New York (1987)). Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler & Milstein, Nature 256:495 (1975), or may be made by recombinant DNA methods (U.S. Pat. No. 4,816,567). The “monoclonal antibodies” may also be isolated from phage libraries generated using the techniques described in McCafferty et al., Nature 348:552-554 (1990), for example.

“Humanized” forms of non-human (e.g., murine) antibodies are specific chimeric immunoglobulins, immunoglobulin chains or fragments thereof (such as Fv, Fab, Fab′, F(ab)2 or other antigen-binding subsequences of antibodies) which contain minimal sequence derived from non-human immunoglobulin. For the most part, humanized antibodies are human immunoglobulins (recipient antibody) in which residues from the complementary determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues that are found neither in the recipient antibody nor in the imported CDR or FR sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also will comprise at least a portion of an immunoglobulin constant region (Fc), typically that of a human immunoglobulin.

“Treatment” refers to both therapeutic treatment and prophylactic or preventative measures. Those in need of treatment include those already with the disorder, as well as those prone to have the disorder or those in which the disorder is to be prevented.

“Mammal” for purposes of treatment refers to any animal classified as a mammal, including humans, domestic and farm animals, and zoo, sports, or pet animals, such as sheep, dogs, horses, cats, cows, etc. In certain instances, the mammal herein is human.

4.3 LcrV Proteins and Polypeptides and Nucleic Acids

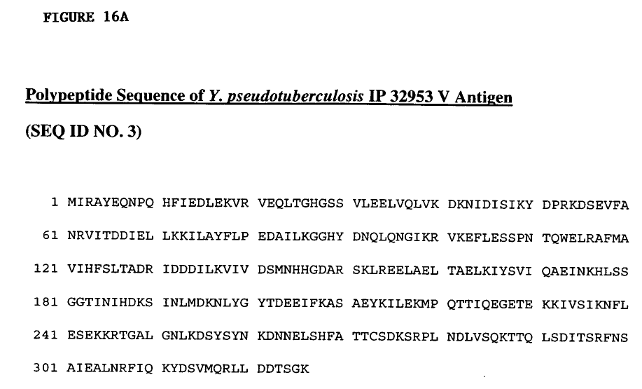

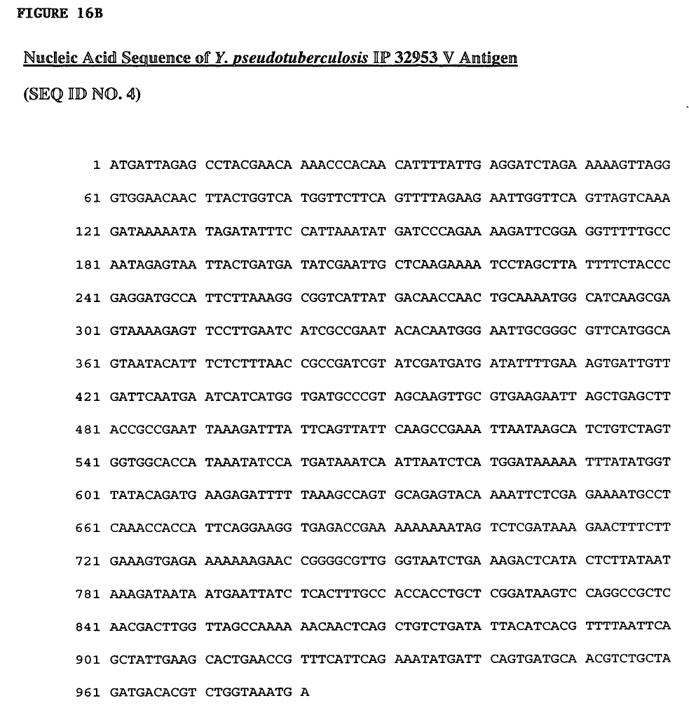

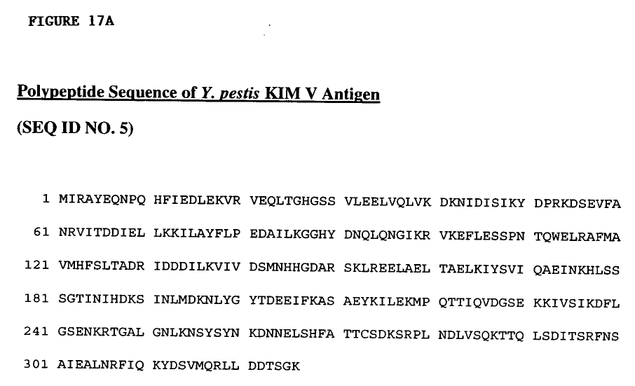

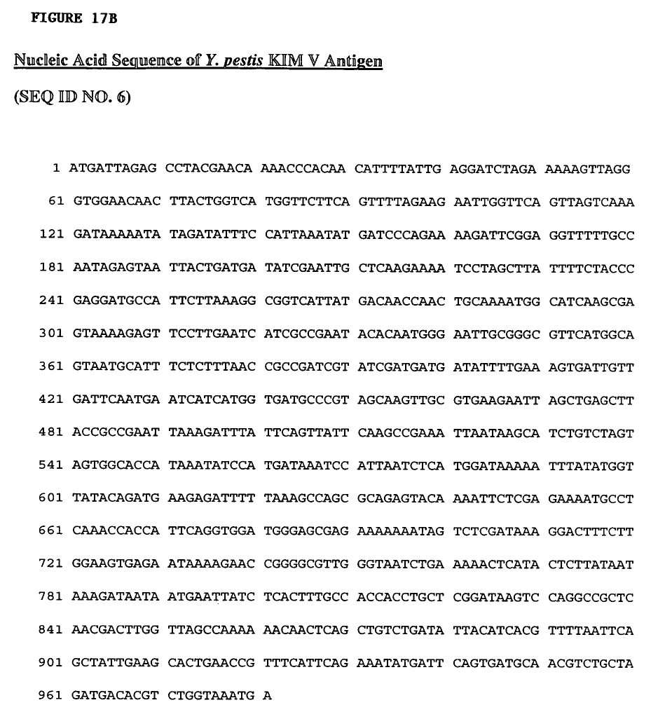

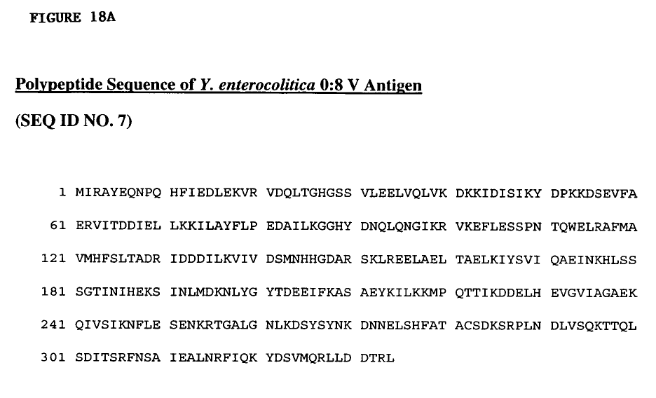

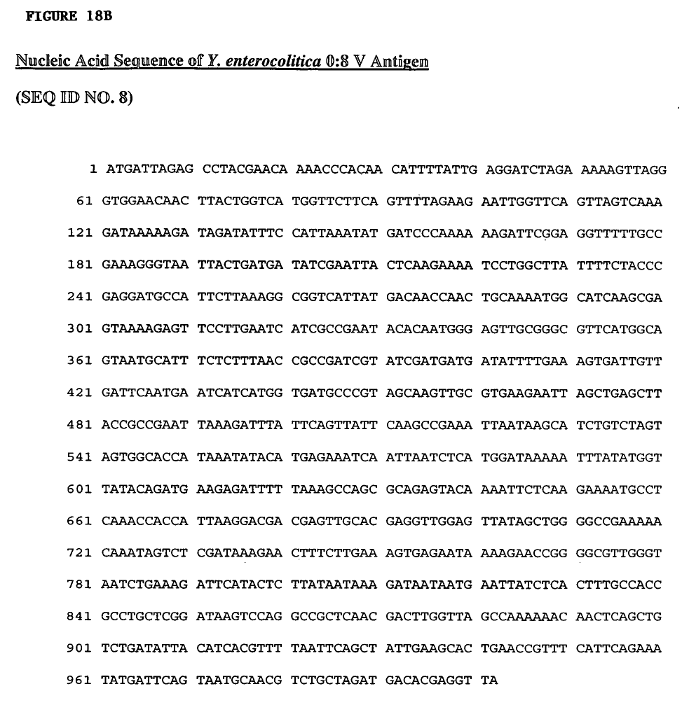

The invention includes LcrV proteins and polypeptides for use in the various embodiments of the present invention. The invention further provides recombinant polynucleotides encoding the modified recombinant LcrV proteins and polypeptides of the invention. The nucleic acid sequences for some non-limiting wild-type Yersinia LcrV proteins include S38727, M57893, BX936399 (Yersinia pseudotuberculosis); AF053946, AF074612, AE017043, AL117189, M26405 (Yersinia pestis ); and AF102990, AF336309, NC 004564 (Yersinia enterocolitis), all of which are incorporated by reference and can be used to prepare a modified LcrV protein of the invention. Further Yersinia DNA and polypeptide sequences of the invention are found in FIGS. 9, 16, 17 and 18 (SEQ ID Nos:1-8).

The proteins of the invention can be prepared by any means known in the art. For example, the proteins can be synthesized in solution or on a solid support in accordance with conventional techniques. Various automatic synthesizers are commercially available and can be used in accordance with known protocols, (see, e.g., Stewart and Young, (1984); Tam et al., (1983); Merrifield, (1986); and Barany and Merrifield (1979). Alternatively, recombinant DNA technology may be employed wherein a nucleotide sequence which encodes a peptide of the invention is inserted into an expression vector, transformed or transfected into an appropriate host cell and cultivated under conditions suitable for expression. In general, in vitro protein production involves transduction with a vector. Methods of immunoaffinity purification for obtaining highly purified LcrV protein or polypeptide immunogen are also known (see, e.g., Vladutiu et al., (1975) 5: 147-59 Prep. Biochem.).

As used in this application, the term “polynucleotide” refers to a nucleic acid molecule that either is recombinant or has been isolated free of total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be RNA, DNA, analogs thereof, or a combination thereof.

In this respect, the term “gene” is used for simplicity to refer to a functional protein, polypeptide, or peptide-encoding unit (including any sequences required for proper transcription, post-translational modification, or localization). As will be understood by those in the art, this functional term includes genomic sequences, cDNA sequences, and smaller engineered gene segments that express, or may be adapted to express, proteins, polypeptides, domains, peptides, fusion proteins, and mutants. A nucleic acid encoding all or part of a native or modified polypeptide may contain a contiguous nucleic acid sequence encoding all or a portion of such a polypeptide of the following lengths: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs.

It also is contemplated that a particular polypeptide from a given species may be represented by natural variants that have slightly different nucleic acid sequences but, nonetheless, encode the same protein as described further herein.

The invention in part, relates to isolated DNA segments and recombinant vectors incorporating DNA sequences that encode a modified LcrV protein. Thus, an isolated DNA segment or vector containing a DNA segment may encode, for example, a modified LcrV protein that is immunogenic but is less immunosuppressant compared to the cognate LcrV protein (protein sequence from which it was derived). The term “recombinant” may be used in conjunction with a polypeptide or the name of a specific polypeptide, and this generally refers to a polypeptide produced from a nucleic acid molecule that has been manipulated in vitro or that is the replicated product of such a molecule.

The invention also includes isolated DNA segments and recombinant vectors incorporating DNA sequences that encode a modified LcrV polypeptide or peptide that can be used to generate an immune response in a subject. These composition can be used as DNA vaccines in some embodiments of the invention.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length being limited by the ease of preparation and use in the intended recombinant DNA protocol. In some cases, a nucleic acid sequence may encode a polypeptide sequence with additional heterologous coding sequences, for example to allow for purification of the polypeptide, transport, secretion, post-translational modification, or for therapeutic benefits such as targetting or efficacy. As discussed above, a tag or other heterologous polypeptide may be added to the modified polypeptide-encoding sequence, wherein “heterologous” refers to a polypeptide that is not the same as the modified polypeptide.

The polynucleotides used in the present invention encompass modified LcrV polypeptides and peptides that may be biologically and functionally equivalent in some aspects to an unmodified LcrV protein but different in others. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein, to decrease immunosuppression caused by the protein, to reduce toxicity effects of the protein in vivo to a subject given the protein, or to increase the efficacy of any treatment involving the protein.

In certain instances, the invention concerns isolated DNA segments and recombinant vectors that include within their sequence a contiguous nucleic acid sequence from that shown in sequences identified herein (and/or incorporated by reference). Such sequences, however, may be modified to yield a protein product whose activity is altered with respect to wild-type, as discussed herein.

It also will be understood that this invention is not limited to the particular nucleic acid and amino acid sequences of these identified sequences. For example, the invention includes modified LcrV proteins that have a deletion of one or more amino acids compared to the unmodified LcrV protein. Deletions may be internal deletions (not encompassing the amino acid at either the 5′ or 3′ end) or they may be terminal deletions. In some cases, a modified LcrV may have multiple regions deleted, including internal and/or terminal regions. Such deletions can be readily constructed by the skilled artisan, including by the methods described in the Examples.

The present invention also concerns DNA vaccines. The vehicle for a DNA segment encoding a protein against which an immune response is desired is well established (see, e.g., U.S. Pat. Nos. 6,821,957, 6,825,029, 6,841,360, 6,846,487, and 6,848,808). Such a vehicle often contains unmethylated immunostimulatory CpG-S motifs, such as those described in U.S. Pat. No. 6,821,957. These motifs serve as a self-adjuvant, and such a polynucleotide can be used with or without other adjuvants, which are discussed infra.

Modified LcrV polypeptides may be encoded by a nucleic acid molecule in a vector. The term “vector” is used to refer to a carrier nucleic acid molecule into which an exogenous nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art can construct a vector through standard recombinant techniques, which are described in Sambrook et al., ((2001) Molecular Cloning A Laboratory Manual, 2nd Ed., ed. by Sambrook, Fritsch and Maniatis (Cold Spring Harbor Laboratory Press: 1989). In addition to encoding a modified LcrV polypeptide, a vector may encode non-LcrV polypeptide sequences such as a tag or targeting molecule. Useful vectors encoding such fusion proteins include pIN vectors, vectors encoding a stretch of histidines, and pGEX vectors, for use in generating glutathione S-transferase (GST) soluble fusion proteins for later purification and separation or cleavage. A targeting molecule is one that directs the modified polypeptide to a particular organ, tissue, cell, or other location in a subject's body.

Vectors of the invention may be used in a host cell to produce a modified LcrV polypeptide that may subsequently be purified for administration to a subject or the vector may be purified for direct administration to a subject for expression of the protein in the subject.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

When the antigenic epitope peptides to be used are relatively short in length (i.e., less than about 50 amino acids), they are often synthesized using standard chemical peptide synthesis techniques. Solid phase synthesis, in which the C-terminal amino acid of the sequence is attached to an insoluble support followed by sequential addition of the remaining amino acids in the sequence, is a useful method for the chemical synthesis of the antigenic epitopes described herein. Techniques for solid phase synthesis are known to those skilled in the art.

Alternatively, the antigenic epitopes described herein are synthesized using recombinant nucleic acid methodology. Generally, this involves creating a nucleic acid sequence that encodes the peptide or polypeptide, placing the nucleic acid in an expression cassette under the control of a particular promoter, expressing the peptide or polypeptide in a host, isolating the expressed peptide or polypeptide and, if required, renaturing the peptide or polypeptide. Techniques sufficient to guide one of skill through such procedures are found in the literature.

While the antigenic epitopes are often joined directly together, one of skill in the art is aware that the antigenic epitopes may be separated by a spacer molecule such as, for example, a peptide, consisting of one or more amino acids. Generally, the spacer will have no specific biological activity other than to join the antigenic epitopes together, or to preserve some minimum distance or other spatial relationship between them. However, the constituent amino acids of the spacer may be selected to influence some property of the molecule such as the folding, net charge, or hydrophobicity.

Once expressed, recombinant peptides, polypeptides and proteins can be purified according to standard procedures known to one of skill in the art, including, but not limited to, ammonium sulfate precipitation, affinity purification, column chromatography, gel electrophoresis and the like. Substantially pure compositions of about 50% to 95% and from 80% to 95% or greater homogeneity are useful as therapeutic agents.

Specific design criteria for the immunomodulatory LcrV proteins and polypeptides are described for example, in Tam (1988) Proc. Natl. Acad. Sci. USA 85:5409-5413, Rizo et al. (1992) Ann. Rev. Biochem., 61:387-418, Cudic et al. (2000) Tetrahedron Lett., 41:4527-4531, and Oomen et al. (2003) J. Mol. Biol., 328:1083-1089. These publications provide design criteria for producing and optimizing synthetic peptides for vaccine and other immunological applications (e.g., providing an anti-inflammatory TLR2 binding site activity) that include use of branched, circular and other constrained polypeptide sequences as well as head to tail tandem repetitive polypeptide sequences and circular versions thereof. Further considerations for design of these LcrV proteins and polypeptides of the invention is provided by the known structure of Further considerations for design of these LcrV proteins and polypeptides of the invention is provided by the known secondary and tertiary structure of Yersinia pestis V-Antigen, which is describe in Derewenda et al. (2004) Structure 12: 301-306.

One of skill in the art will recognize that after chemical synthesis, biological expression or purification, the LcrV antigenic peptide epitopes, polypeptides and proteins of the invention may possess a conformation substantially different than the native conformations of the constituent peptides. In this case, it is often necessary to denature and reduce the polypeptide and then to cause the polypeptide to refold into a favored conformation. Methods of reducing and denaturing proteins and inducing refolding are well known to those of skill in the art.

4.4 Antibodies to LcrV Protein and Polypeptides

The invention also provides antibodies directed against LcrV protein or polypeptide for use in treating and detecting infectious disease, e.g. by Yersinia species including Y. pestis. Such antibodies include polyclonal and monoclonal antibodies, and recombinant and humanized antibodies, and LcrV-binding fragments thereof. Accordingly, the term “antibody” is used in the broadest sense and specifically covers single anti-LcrV protein or polypeptide monoclonal and polyclonal antibides as well as anti-LcrV protein or polypeptide antibody fragments (e.g, Fab, F(ab)2 and Fv) and anti-LcrV protein or polypeptide antibody compositions with polyepitopic specificity (including binding and non-binding antibodies).

LcrV protein or polypeptide or anti-LcrV protein or polypeptide monoclonal antibodies or fragments thereof mean in principle all immunoglobulin classes such as IgM, IgG, IgD, IgE, IgA or their subclasses such as the IgG subclasses or mixtures thereof. IgG and its subclasses are, such as IgG1, IgG2, IgG2a, IgG2b, IgG3 or IgGM. The IgG subtypes IgG1/kappa and IgG 2b/kapp are included as embodiments. Fragments which may be mentioned are all truncated or modified antibody fragments with one or two antigen-complementary binding sites which show high binding and binding activity toward mammalian LcrV protein or polypeptide, such as parts of antibodies having a binding site which corresponds to the antibody and is formed by light and heavy chains, such as Fv, Fab or F(ab′)2 fragments, or single-stranded fragments. Truncated double-stranded fragments such as Fv, Fab or F(ab′)2 are. These fragments can be obtained, for example, by enzymatic means by eliminating the Fc part of the antibody with enzymes such as papain or pepsin, by chemical oxidation or by genetic manipulation of the antibody genes. It is also possible and advantageous to use genetically manipulated, non-truncated fragments. The anti-LcrV protein or polypeptide antibodies or fragments thereof can be used alone or in mixtures.

The novel antibodies, antibody fragments, mixtures or derivatives thereof advantageously have a binding affinity for LcrV protein or polypeptide with a dissociation constant value within a log-range of from about 1×10⁻¹¹ M (0.01 nM) to about 1×10⁻⁸ M (10 nM), or about 1×10⁻¹⁰ M (0.1 nM) to about 3×10⁹⁹ M (3 nM).

The antibody genes for the genetic manipulations can be isolated, for example from hybridoma cells, in a manner known to the skilled worker. For this purpose, antibody-producing cells are cultured and, when the optical density of the cells is sufficient, the MRNA is isolated from the cells in a known manner by lysing the cells with guanidinium thiocyanate, acidifying with sodium acetate, extracting with phenol, chloroform/isoamyl alcohol, precipitating with isopropanol and washing with ethanol. cDNA is then synthesized from the mRNA using reverse transcriptase. The synthesized cDNA can be inserted, directly or after genetic manipulation, for example by site-directed mutagenesis, introduction of insertions, inversions, deletions or base exchanges, into suitable animal, fungal, bacterial or viral vectors and be expressed in appropriate host organisms. Useful bacterial or yeast vectors include, but are not limited to, pBR322, pUC18/19, pACYC184, lambda or yeast mu vectors for the cloning of the genes and expression in bacteria such as E. coli or in yeasts such as S. cerevisiae.

The invention furthermore relates to cells that synthesize LcrV protein or polypeptide antibodies. These include animal, fungal, bacterial cells or yeast cells after transformation as mentioned above. They are advantageously hybridoma cells or trioma cells. These hybridoma cells can be produced, e.g., in a known manner from animals immunized with LcrV protein or polypeptide and isolation of their antibody-producing B cells, selecting these cells for LcrV protein or polypeptide-binding antibodies and subsequently fusing these cells to, for example, human or animal, for example, mouse mylemoa cells, human lymphoblastoid cells or heterohybridoma cells (see, e.g., Koehler et al. (1975) Nature 256: 496), or by infecting these cells with appropriate viruses to produce immortalized cell lines. The hybridoma cell lines of the invention secrete antibodies of the IgG type. The binding of the mAb antibodies of the invention, bind with high affinity to LcrV protein or polypeptide.

The invention further includes derivates of these anti-LcrV protein or polypeptide antibodies, which usefully retain their LcrV protein or polypeptide-binding activity while altering one or more other properties related to their use as a pharmaceutical agent, e.g., serum stability or efficiency of production. Examples of such anti-LcrV protein or polypeptide antibody derivatives include, but are not limited to, peptides, peptidomimetics derived from the antigen-binding regions of the antibodies, and antibodies, fragments or peptides bound to solid or liquid carriers such as polyethylene glycol, glass, synthetic polymers such as polyacrylamide, polystyrene, polypropylene, polyethylene or natural polymers such as cellulose, Sepharose or agarose, or conjugates with enzymes, toxins or radioactive or nonradioactive markers such as ³H, ¹²³I, ¹²⁵I, ¹³¹I, ³²P, ³⁵S, ¹⁴C, ⁵¹Cr, ³⁶Cl, ⁵⁷Co, ⁵⁵Fe, ⁵⁹Fe, ⁹⁰Y, ⁹⁹mTc (metastable isomer of Technetium 99), ⁷⁵Se, or antibodies, fragments or peptides covalently bonded to fluorescent/chemiluminescent labels such as rhodamine, fluorescein, isothiocyanate, phycoerythrin, phycocyanin, fluorescamine, metal chelates, avidin, streptavidin or biotin.

The novel antibodies, antibody fragments, mixtures and derivatives thereof can be used directly, after drying (e.g., freeze drying), after attachment to the abovementioned carriers or after formulation with other pharmaceutical active and ancillary substances for producing pharmaceutical preparations. Non-limiting examples of active and ancillary substances which may be mentioned are other antibodies, antimicrobial active substances with a microbiocidal or microbiostatic action such as antibiotics in general or sulfonamides, antitumor agents, water, buffers, salines, alcohols, fats, waxes, inert vehicles or other substances customary for parenteral products, such as amino acids, thickeners or sugars. These pharmaceutical preparations are used to control diseases, usefully to control Yersinia infection.

The anti-LcrV protein or polypeptide antibodies of the invention can be administered as determined by a physician, e.g., orally or parenterally subcutaneously, intramuscularly, intravenously or interperitoneally.

The antibodies, antibody fragments, mixtures or derivatives thereof can be used in therapy or diagnosis directly or after coupling to solid or liquid carriers, enzymes, toxins, radioactive or nonradioactive labels or to fluorescent/chemiluminescent labels as described above. LcrV protein or polypeptide can be detected on a wide variety of cell types, including particularly eoplastic cells. The Yersinia LcrV protein or polypeptide monoclonal antibody of the present invention may be obtained as follows. Those of skill in the art will recognize that other equivalent procedures for obtaining LcrV protein or polypeptide antibodies are also available and are included in the invention.

First, a mammal is immunized with Yersinia LcrV protein or polypeptide. The mammal used for raising anti-Yersinia LcrV protein or polypeptide antibody is not restricted and may be a primate, a rodent such as mouse, rat or rabbit, bovine, sheep, goat or dog.

Next, antibody-producing cells such as spleen cells are removed from the immunized animal and are fused with myeloma cells. The myeloma cells are well-known in the art (e.g., p3x63-Ag8-653, NS-0, NS-1 or P3U1 cells may be used). The cell fusion operation may be carried out by methods well-known in the art.

The cells, after being subjected to the cell fusion operation, are then cultured in, e.g., HAT selection medium so as to select hybridomas. Hybridomas, which produce antihuman monoclonal antibodies, are then screened. This screening may be carried out, for example, by sandwich ELISA (enzyme-linked immunosorbent assay) or the like in which the produced monoclonal antibodies are bound to the wells to which Yersinia LcrV protein or polypeptide is immobilized. In this case, as the secondary antibody, an antibody specific to the immunoglobulin of the immunized animal, which is labeled with an enzyme such as peroxidase, alkaline phosphatase, glucose oxidase, beta-D-galactosidase or the like, may be employed. The label may be detected by reacting the labeling enzyme with its substrate and measuring the generated color. As the substrate, 3,3-diaminobenzidine, 2,2-diaminobis-o-dianisidine, 4-chloronaphthol, 4-aminoantipyrine, o-phenylenediamine or the like may be produced.

By the above-described operation, hybridomas, which produce anti-Yersinia LcrV protein or polypeptide antibodies are selected. The selected hybridomas are then cloned by the conventional limiting dilution method or soft agar method. If desired, the cloned hybridomas may be cultured on a large scale using a serum-containing or a serum free medium, or may be inoculated into the abdominal cavity of mice and recovered from ascites.

Those selected anti-Yersinia LcrV protein or polypeptide monoclonal antibodies that have an ability to bind LcrV protein or polypeptide are then further analyzed and manipulated.

The monoclonal antibodies herein further include hybrid and recombinant antibodies produced by splicing a variable (including hypervariable) domain of an anti-LcrV protein or polypeptide antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)2, and Fv), so long as they exhibit the desired biological activity. (See, e.g., U.S. Pat. No. 4,816,567 and Mage & Lamoyi, in Monoclonal Antibody Production Techniques and Applications, pp. 79-97 (Marcel Dekker, Inc.), New York (1987)).

Thus, the term “monoclonal” indicates that the character of the antibody obtained is from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256:495 (1975), or may be made by recombinant DNA methods (U.S. Pat. No. 4,816,567). The “monoclonal antibodies” may also be isolated from phage libraries generated using the techniques described in McCafferty et al., Nature 348:552-554 (1990), for example.

“Humanized” forms of non-human (e.g., murine) antibodies are specific chimeric immunoglobulins, immunoglobulin chains or fragments thereof (such as Fv, Fab, Fab′, F(ab)2 or other antigen-binding subsequences of antibodies) which contain minimal sequence derived from non-human immunoglobulin. For the most part, humanized antibodies are human immunoglobulins (recipient antibody) in which residues from the complementary determining regions (CDRs) of the recipient antibody are replaced by residues from the CDRs of a non-human species (donor antibody) such as mouse, rat or rabbit having the desired specificity, affinity and capacity. In some instances, Fv framework region (FR) residues of the human immunoglobulin are replaced by corresponding non-human FR residues. Furthermore, the humanized antibody may comprise residues that are found neither in the recipient antibody nor in the imported CDR or FR sequences. These modifications are made to further refine and optimize antibody performance. In general, the humanized antibody comprises substantially all of at least one, and typically two, variable domains, in which all or substantially all of the CDR regions correspond to those of a non-human immunoglobulin and all or substantially all of the FR residues are those of a human immunoglobulin consensus sequence. The humanized antibody optimally also comprises at least a portion of an immunoglobulin constant region (Fc), typically that of a human immunoglobulin.

Methods for humanizing non-human antibodies are well known in the art. Generally, a humanized antibody has one or more amino acid residues introduced into it from a source, which is non-human. These non-human amino acid residues are often referred to as “import” residues, which are typically taken from an “import” variable domain. Humanization can be essentially performed following the method of Winter and co-workers (Jones et al., (1986) Nature 321: 522-525; Riechmann et al., (1988) Nature, 332: 323-327; and Verhoeyen et al., (1988) Science 239: 1534-1536), by substituting rodent CDRs or CDR sequences for the corresponding sequences of a human antibody. Accordingly, such “humanized” antibodies are chimeric antibodies, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., (1993) J. Immunol., 151:2296; and Chothia and Lesk (1987) J. Mol. Biol., 196:901). Another method uses a particular framework derived from the consensus sequence of all human antibodies of a particular subgroup of light or heavy chains. The same framework may be used for several different humanized antibodies (Carter et al., (1992) Proc. Natl. Acad. Sci. (USA), 89: 4285; and Presta et al., (1993) J. Immnol., 151:2623).

It is further important that antibodies be humanized with retention of high affinity for the antigen and other favorable biological properties. To achieve this goal, according to a method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

Human antibodies directed against LcrV protein or polypeptide are also included in the invention. Such antibodies can be made, for example, by the hybridoma method. Human myeloma and mouse-human heteromyeloma cell lines for the production of human monoclonal antibodies have been described, for example, by Kozbor (1984) J. Immunol., 133, 3001; Brodeur, et al., Monoclonal Antibody Production Techniques and Applications, pp. 51-63 (Marcel Dekker, Inc., New York, 1987); and Boerner et al., (1991) J. Immunol., 147:86-95. Specific methods for the generation of such human antibodies using, for example, phage display, transgenic mouse technologies and/or in vitro display technologies, such as ribosome display or covalent display, have been described (see Osbourn et al. (2003) Drug Discov. Today 8: 845-51; Maynard and Georgiou (2000) Ann. Rev. Biomed. Eng. 2: 339-76; and U.S. Pat. Nos.: 4,833,077; 5,811,524; 5,958,765; 6,413,771; and 6,537,809.

Transgenic animals (e.g., mice) are capable, upon immunization, of producing a full repertoire of human antibodies in the absence of endogenous immunoglobulin production. For example, the homozygous deletion of the antibody heavy-chain joining region (JH) gene in chimeric and germ-line mutant mice results in complete inhibition of endogenous antibody production. Transfer of the human germ-line immunoglobulin gene array in such gem-line mutant mice results in the production of human antibodies upon antigen challenge (see, e.g., Jakobovits et al., (1993) Proc. Natl. Acad. Sci. (USA), 90: 2551).

Alternatively, phage display technology (McCafferty et al., (1990) Nature, 348: 552-553) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats (for review see, e.g., Johnson et al., (1993) Curr. Opin. in Struct. Bio., 3:564-571). Several sources of V-gene segments can be used for phage display. (See, e.g., Clackson et al., ((1991) Nature, 352: 624-628). A repertoire of V genes from unimmunized human donors can be constructed and antibodies to a diverse array of antigens (including self-antigens) can be isolated (see, e.g., Marks et al., ((1991) J. Mol. Biol., 222:581-597, or Griffith et al., (1993) EMBO J., 12:725-734).

In a natural immune response, antibody genes accumulate mutations at a high rate (somatic hypermutation). Some of the changes introduced will confer higher affinity, and B cells displaying high-affinity surface immunoglobulin are replicated and differentiated during subsequent antigen challenge. This natural process can be mimicked by employing the technique known as “chain shuffling” (see Marks et al., (1992) Bio/Technol., 10:779-783). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires has been described by Waterhouse et al., ((1993) Nucl. Acids Res., 21:2265-2266).

Gene shuffling can also be used to derive human LcrV antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e., the epitope governs (imprints) the choice of partner. When the process is repeated in order to replace the remaining rodent V domain, a human antibody is obtained (see PCT WO 93/06213). Unlike traditional humanization of rodent antibodies by CDR grafting, this technique provides completely human antibodies, which have no framework or CDR residues of rodent origin.

By using the above-described monoclonal antibody of the present invention, Yersinia LcrV protein or polypeptide in a sample can be detected or quantified. The detection or quantification of the Yersinia LcrV protein or polypeptide in a sample can be carried out by any method known in the art, e.g., by an immunoassay utilizing the specific binding reaction between the monoclonal antibody of the present invention and Yersinia LcrV protein or polypeptide. Various immunoassays are well-known in the art and any of them can be employed. Non-limiting examples of the immunoassays include sandwich method employing the monoclonal antibody and another monoclonal antibody as primary and secondary antibodies, respectively, sandwich methods employing the monoclonal antibody and a polyclonal antibody as primary and secondary antibodies, staining methods employing gold colloid, agglutination method, latex method and chemical luminescence. Among these, especially is sandwich ELISA. As is well-known, in this method, a primary LcrV antibody is immobilized on, for example, the inner wall of a well and then a sample is reacted with the immobilized primary antibody. After washing, a secondary antibody is reacted with the antigen-antibody complex immobilized in the well. After washing, the immobilized secondary antibody is quantified. As the primary antibody, an antibody specifically reacts with Yersinia LcrV protein or polypeptide is usefully employed.

The quantification of the secondary antibody may be carried out by reacting a labeled antibody (e.g., enzyme-labeled antibody) specific to the immunoglobulin of the animal from which the secondary antibody was obtained with the secondary antibody, and then measuring the label. Alternatively, a labeled (e.g., enzyme-labeled) antibody is used as the secondary antibody and the quantification of the secondary antibody may be carried out by measuring the label on the secondary antibody.

4.5 LcrV Protein and Polypeptide Vaccines

Immunological compositions, including vaccines, and other pharmaceutical compositions containing the LcrV protein or polypeptide protein, or portions thereof, are included within the scope of the present invention. One or more of the LcrV protein or polypeptide, or active or antigenic fragments thereof, or fusion proteins thereof can be formulated and packaged, alone or in combination with other antigens, using methods and materials known to those skilled in the art for vaccines. The immunological response may be used therapeutically or prophylactically and may provide antibody immunity or cellular immunity, such as that produced by T lymphocytes.

To enhance immunogenicity, the proteins may be conjugated to a carrier molecule. Suitable immunogenic carriers include, but are not limiting to, proteins, polypeptides or peptides such as albumin, hemocyanin, thyroglobulin and derivatives thereof, particularly bovine serum albumin (BSA) and keyhole limpet hemocyanin (KLH), polysaccharides, carbohydrates, polymers, and solid phases. Other protein derived or non-protein derived substances are known to those skilled in the art. An immunogenic carrier typically has a molecular mass of at least 1,000 Daltons, usefully greater than 10,000 Daltons. Carrier molecules often contain a reactive group to facilitate covalent conjugation to the hapten. The carboxylic acid group or amine group of amino acids or the sugar groups of glycoproteins are often used in this manner. Carriers lacking such groups can often be reacted with an appropriate chemical to produce them. Typically, an immune response is produced when the immunogen is injected into animals such as mice, rabbits, rats, goats, sheep, guinea pigs, chickens, and other animals. Alternatively, a multiple antigenic peptide comprising multiple copies of the protein or polypeptide, or an antigenically or immunologically equivalent polypeptide, may be sufficiently antigenic to improve immunogenicity without the use of a carrier.

The LcrV protein, or polypeptide protein or portions thereof, such as consensus or variable sequence amino acid motifs, or combination of proteins, may be administered with an adjuvant in an amount effective to enhance the immunogenic response against the conjugate. One adjuvant widely used in humans has been alum (aluminum phosphate or aluminum hydroxide). Saponin and its purified component Quil A, Freund's complete adjuvant and other adjuvants used in research and veterinary applications are also available. Chemically defined preparations such as muramyl dipeptide, monophosphoryl lipid A, phospholipid conjugates (see, e.g., Goodman-Snitkoff et al. (1991) J. Immunol. 147:410-415), encapsulation of the conjugate within a proteoliposome (see, e.g., Miller et al. (1992) J. Exp. Med. 176:1739-1744) and encapsulation of the protein in lipid vesicles such as Novasome™ lipid vesicles (Micro Vescular Systems, Inc., Nashua, N.H.) may also be useful.

A variety of other adjuvants known to one of ordinary skill in the art may be administered in conjunction with the protein in the vaccine composition. Such adjuvants include, but are not limited to, polymers, co-polymers such as polyoxyethylene-polyoxypropylene copolymers, including block co-polymers; polymer P1005; Freund's complete adjuvant (for animals); Freund's incomplete adjuvant; sorbitan monooleate; squalene; CRL-8300 adjuvant; alum; QS 21, muramyl dipeptide; CpG oligonucleotide motifs and combinations of CpG oligonucleotide motifs; trehalose; bacterial extracts, including mycobacterial extracts; detoxified endotoxins; membrane lipids; or combinations thereof. In addition, the present invention provides a composition comprising the LcrV protein or polypeptide protein or polypeptide fragment of the invention in combination with a suitable adjuvant. Such a composition can be in a pharmaceutically acceptable carrier, as described herein. As used herein, “adjuvant” or “suitable adjuvant” describes a substance capable of being combined with the LcrV protein or polypeptide protein or polypeptide to enhance an immune response in a subject without deleterious effect on the subject. A suitable adjuvant can be, but is not limited to, for example, an immunostimulatory cytokine, SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Other suitable adjuvants are well known in the art and include QS-21, Freund's adjuvant (complete and incomplete), alum, aluminum phosphate, aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion. The adjuvant, such as an immunostimulatory cytokine can be administered before the administration of the LcrV protein or polypeptide protein or LcrV protein or polypeptide-encoding nucleic acid, concurrent with the administration of the LcrV protein or polypeptide protein or LcrV protein or polypeptide-encoding nucleic acid or up to five days after the administration of the LcrV protein or polypeptide protein or LcrV protein or polypeptide-encoding nucleic acid to a subject. QS-21, similarly to alum, complete Freund's adjuvant, SAF, etc., can be administered within hours of administration of the fusion protein.

The invention may also utilize combinations of adjuvants, such as immunostimulatory cytokines co-administered to the subject before, after or concurrent with the administration of the LcrV protein or polypeptide protein or LcrV protein or polypeptide-encoding nucleic acid. For example, combinations of adjuvants, such as immunostimulatory cytokines, can consist of two or more of immunostimulatory cytokines of this invention, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants may be determined by measuring the immune response directed against the LcrV protein or polypeptide with and without the adjuvant or combination of adjuvants, using standard procedures, as described herein.

LcrV protein or polypeptide polypeptide subsequences, or a corresponding nucleic acid sequence that encodes them in the case of DNA vaccines, are selected so as to be highly immunogenic. The principles of antigenicity for the purpose of producing anti-LcrV protein or polypeptide vaccines apply also to the use of LcrV protein or polypeptide polypeptide sequences for use as immunogens for generating anti-LcrV protein or polypeptide polyclonal and monoclonal antibodies for use in the LcrV protein or polypeptide-based diagnostics and therapeutics described herein.

Computer assisted algorithms for predicting polypeptide subsequence antigenicity are widely available. For example “Antigenic” looks for potential antigenic regions using the method of Kolaskar (see Kolaskar and Tongaonkar (1990) FEBS Letters 276:172-174).

Another method for determining antigenicity of a polypeptide subsequence is the algorithm of Hopp and Woods ((1981) Proc. Natl. Acad. Sci. 86: 152-6). There are publicly available web sites for Hopp and Woods algorithm analysis of a user-input polypeptide sequence and convenient graphical output of the resulting analysis (see, e.g., http://hometown.aol.com/_ht_a/lucatoldo/myhomepage/JaMBW/3/1/7/).

Further design criteria for the immunomodulatory LcrV proteins and polypeptides are also known (see, e.g., Tam (1988) Proc. Natl. Acad. Sci. USA 85:5409-5413, Rizo et al. (1992) Annu. Rev. Biochem., 61:387-418, Cudic et al. (2000) Tetrahedron Lett., 41:4527-4531, and Oomen et al. (2003) J. Mol. Biol., 328:1083-1089). These known methods provide design criteria for producing and optimizing synthetic peptides for vaccine and other immunological applications (e.g., providing an anti-inflammatory TLR2 binding site activity) that include use of branched, circular and other constrained polypeptide sequences as well as head to tail tandem repetitive polypeptide sequences and circular versions thereof. Further considerations for design of these LcrV proteins and polypeptides of the invention is provided by the known structure of Further considerations for design of these LcrV proteins and polypeptides of the invention is provided by the known secondary and tertiary structure of Yersinia pestis V-Antigen (see, e.g., Derewenda et al. (2004) Structure 12: 301-306).

Furthermore, the present invention provides a composition comprising the LcrV protein or polypeptide protein or LcrV protein or polypeptide-encoding nucleic acid and an adjuvant, such as an immunostimulatory cytokine or a nucleic acid encoding an adjuvant, such as an immunostimulatory cytokine. Such a composition can be in a pharmaceutically acceptable carrier, as described herein. The immunostimulatory cytokine used in this invention can be, but is not limited to, GM/CSF, interleukin-2, interleukin-12, interferon-gamma, interleukin-4, tumor necrosis factor-alpha, interleukin-1, hematopoietic factor flt3L, CD40L, B7.1 con-stimulatory molecules and B7.2 co-stimulatory molecules.

The term “vaccine” as used herein includes DNA vaccines in which the nucleic acid molecule encoding LcrV protein or polypeptide or antigenic portions thereof, such as any consensus or variable sequence amino acid motif, in a pharmaceutical composition is administered to a patient. For genetic immunization, suitable delivery methods known to those skilled in the art include direct injection of plasmid DNA into muscles (Wolff et al. (1992) Hum. Mol. Genet. 1:363), delivery of DNA complexed with specific protein carriers (Wu et al. (1989) J. Biol. Chem. 264:16985, coprecipitation of DNA with calcium phosphate (Benvenisty and Reshef (1986) Proc. Natl. Acad. Sci. 83:9551), encapsulation of DNA in liposomes (Kaneda et al. (1989) Science 243:375,), particle bombardment (Tang et al., (1992) Nature 356:152, and Eisenbraun et al. (1993) DNA Cell Biol. 12:791), and in vivo infection using cloned retroviral vectors (Seeger et al. (1984) Proc. Natl. Acad. Sci. 81:5849).

According to the invention, the vaccine can be a polynucleotide which comprises contiguous nucleic acid sequences capable of being expressed to produce a LcrV protein or polypeptide or immunostimulant gene product upon introduction of said polynucleotide into eukaryotic tissues in vivo. The encoded gene product either acts as an immunostimulant or as an antigen capable of generating an immune response. Thus, the nucleic acid sequences in this embodiment encode an immunogenic epitope, and optionally a cytokine or a T-cell costimulatory element, such as a member of the B7 family of proteins.

There are advantages to immunization with a LcrV gene rather than its gene product include the following. First, is the relative simplicity with which native or nearly native antigen can be presented to the immune system. Mammalian LcrV proteins expressed recombinantly in bacteria, yeast, or even mammalian cells may require extensive treatment to ensure appropriate antigenicity. A second advantage of DNA immunization is the potential for the immunogen to enter the MHC class I pathway and evoke a cytotoxic T cell response (see, e.g., Montgomery, et al. (1997) Cell Mol Biol. 43(3):285-92; and Ulmer, J. et al. (1997) Vaccine 15(8):792-794). Cell-mediated immunity is important in controlling infection. Since DNA immunization can evoke both humoral and cell-mediated immune responses, its advantage may be that it provides a relatively simple method to survey a large number of LcrV protein or polypeptide genes and gene fragments for their vaccine potential.

The invention also includes known methods of preparing and using vaccines in conjunction with chemokines for use in treating or preventing infectious disease. Chemokines are a group of usually small secreted proteins (7-15 kDa) induced by inflammatory stimuli and are involved in orchestrating the selective migration, diapedesis and activation of blood-born leukocytes that mediate the inflammatory response (see Wallack (1993) Ann. New York Acad. of Sci. 178). Chemokines mediate their function through interaction with specific cell surface receptor proteins. At least four chemokine subfamilies have been identified as defined by a cysteine signature motif, termed CC, CXC, C and CX₃ C, where C is a cysteine and X is any amino acid residue. Structural studies have revealed that at least both CXC and CC chemokines share very similar tertiary structure (monomer), but different quaternary structure (dimer). For the most part, conformational differences are localized to sections of loop or the N-terminus. In the instant invention, for example, a Yersinia LcrV protein or polypeptide polypeptide sequence (such as that shown in FIG. 9A), or polypeptide fragment thereof, and a chemokine sequence are fused together and used in an immunizing vaccine. The chemokine portion of the fusion can be a human monocyte chemotactic protein-3, a human macrophage-derived chemokine or a human SDF-1 chemokine. The LcrV protein or polypeptide portion of the fusion is, usefully, a portion shown in routine screening to have a strong antigenic potential.

Additionally, the invention includes vaccines comprising an LcrV protein or polypeptide in combination with another component such as lipopoly-saccharinae or a second Yersinia polypeptide.

For example, some vaccines of the invention include an F1 antigen which is known to provide protective immunity. The F1 antigen, which provides a surface capsule function, provided protective immunity when injected at a dose of 1 μg of recombinant protein produced in E. coli and challenged with an LD₅₀ dose of 106 cfu of Y. pestis strain NM77-538 (LD₅₀ (i.p.)=1.8×10² cfu (see, e.g., Simpson et al. (1990); see also Williamson et al. (1995) FEMS Immunol. Med. Microbiol., 12:223-230; Andrews et al. (1996) Infect. Immun., 64:2180-2187; Andrews et al. (1999) Infect. Immun., 67:1533-1537).

The vaccines of the invention may also include a YopD antigen. The YopD antigen, which provides a Type III system-translocation Yop function, provided protective immunity when injected at a dose of 3×30 μg of recombinant protein produced in E. coli, when given with Ribi R-730 adjuvant, and challenged with an LD₅₀ dose of 140 cfu of Y. pestis strain C092 (Andrews et al. (1999) Infect. Immun., 67:1533-1537).

The vaccines of the invention may also include a YopH antigen (Infect. Immun., 67:1533-1537). In addition, the vaccines of the invention also include a YopE antigen, which provides a Type III system-cytotoxin effector Yop function (see, e.g., Andrews et al. (1999) Infect. Immun., 67:1533-1537); Leary et al. (1999) Microb. Pathog., 26:159-169). The vaccines of the invention may also include a YopN antigen. Protective immunity studies of the YopN antigen, which provides a component that regulates a Yop release function, have been described (Andrews et al. (1999) Infect. Immun., 67:1533-1537; see also Leary et al. (1999) Microb. Pathog., 26:159-169). In addition, the vaccines of the invention may also include a YopK antigen. The YopK antigen, which provides a component that regulates a Yop release function, has been investigated in protective immunity studies (Andrews et al. (1999) Infect. Immun., 67:1533-1537; see also Leary et al. (1999) Microb. Pathog., 26:159-169). The vaccines of the invention may further include a YopM antigen, which provides a Type III system-effector Yop function. Protective immunity studies of the YopM antigen have been reported (Nemeth, J. Straley (1997) Infect. Immun., 65:924-930; Andrews et al. (1999) Infect. Immun., 67:1533-1537).

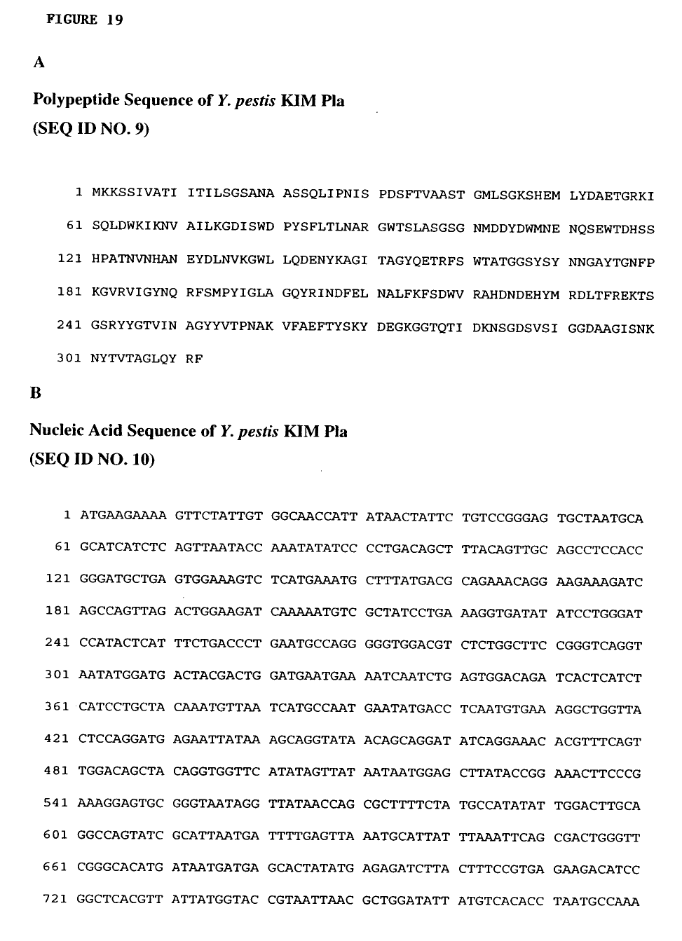

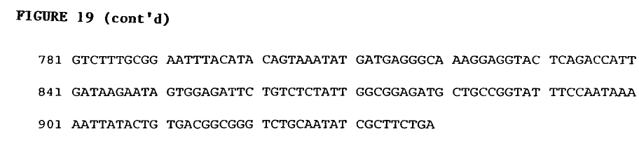

The vaccines of the present invention may further include a Yersinia Plasminogen activator (Pla) antigen. Pla is a surface serine preatease that activates plaminogen and inactivates a2-antiplasmin and enhances adherence to extracellular matrix and laminin and thereby enhances invasion of nonphagocytic cells. Exemplary non-limiting Pla polypeptide and nucleic acid sequences for use in the invention are shown in FIG. 19.

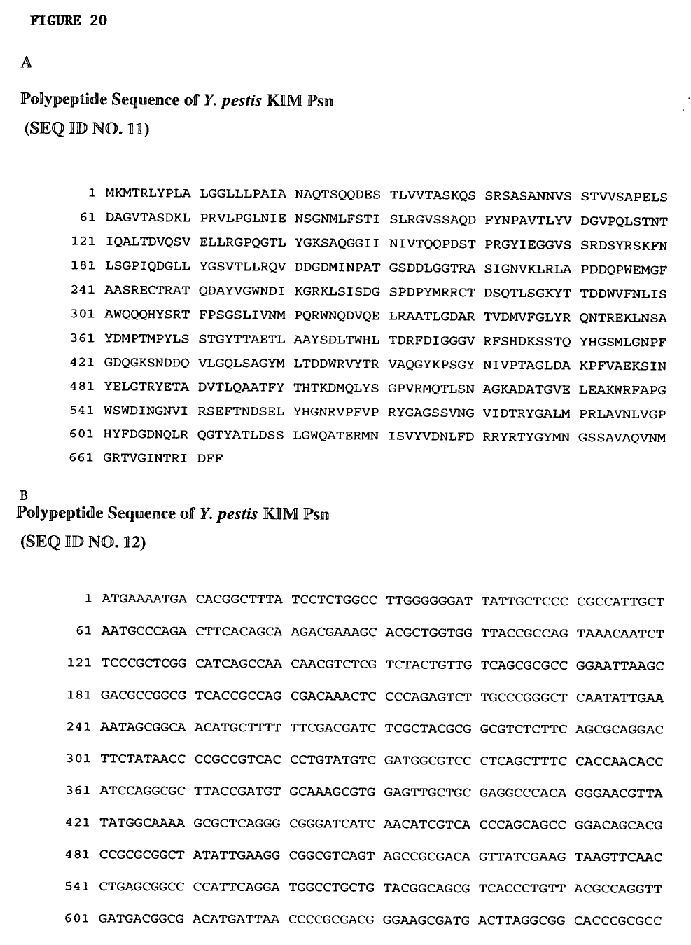

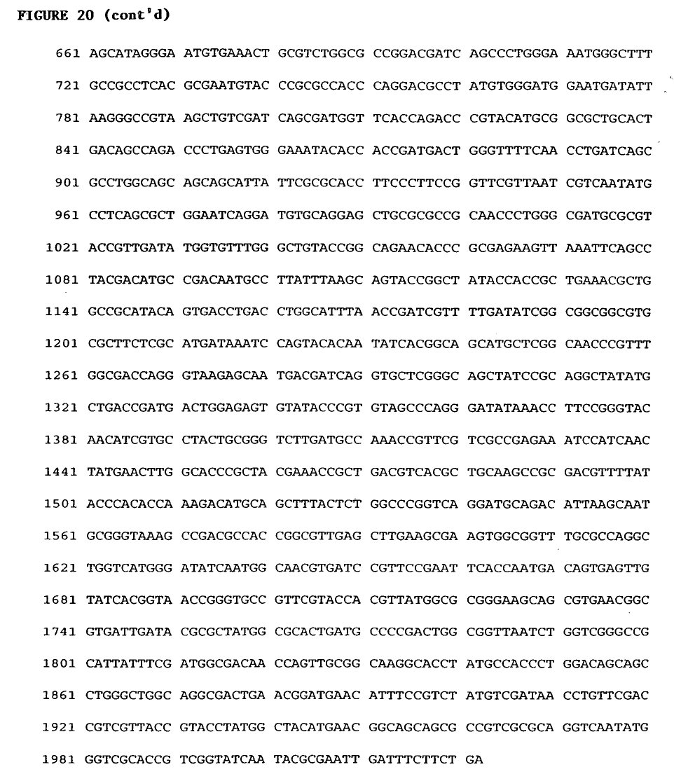

The vaccines of the present invention may further include a Yersinia Psn antigen. Exemplary Psn polypeptide and nucleic acid sequences for use in the invention are shown in FIG. 20.

The vaccines of the present invention may be administered to humans, especially individuals traveling to regions where Yersinia pestis infection is present, and also to inhabitants of those regions. The optimal time for administration of the vaccine as described below is about one to three months before the initial infection. However, the vaccine may also be administered after initial infection to ameliorate disease progression, or after initial infection to treat the disease.

4.6 Pharmaceutical Formulations and Methods of Administration and Treatment

The present invention provides for both prophylactic and therapeutic methods of treating infectious disease and inflammation. Subjects at risk for such a disease can be identified by a diagnostic or prognostic assay, e.g., as described herein. Administration of an LcrV-related a prophylactic agent, e.g., an LcrV protein or polypeptide, or an anti-LcrV protein or polypeptide antibody, can occur prior to the manifestation of symptoms characteristic of the infectious or inflammatory disease, such that development of the disease is prevented or, alternatively, delayed in its progression. In general, the prophylactic or therapeutic methods comprise administering to the subject an effective amount of a compound which comprises a LcrV protein or polypeptide that is capable of binding to cell surface TLR-2 and/or IFN-gamma receptor, and upregulating anti-inflammatory cytokine IL-10 or an anti-LcrV protein or polypeptide antibody that is capable of inhibiting a Yersinia infection.

Toxicity and therapeutic efficacy of such compounds can be determined by standard pharmaceutical procedures in cell cultures or experimental animals, e.g., for determining the LD₅₀ (The Dose Lethal To 50% Of The Population) and The ED₅₀ (the dose therapeutically effective in 50% of the population) (see Remmington's Pharmaceutical Sciences, Meade Publishing Co., Easton, Pa.). The dose ratio between toxic and therapeutic effects is the therapeutic index and it can be expressed as the ratio LD₅₀/ED₅₀. Compounds that exhibit large therapeutic induces are particularly useful. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies usefully within a range of circulating concentrations that include the ED₅₀ with little or no toxicity. The dosage may vary within this range depending upon the dosage form employed and the route of administration utilized. For any LcrV-related compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically acceptable carriers or excipients, as described above. Thus, the compounds and their physiologically acceptable salts and solvates may be formulated for administration by, for example, injection, inhalation or insulation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remington's Pharmaceutical Sciences, Meade Publishing Co., Easton, Pa. For systemic administration, injection is particularly useful, including intramuscular, intravenous, intraperitoneal, and subcutaneous. For injection, the compounds of the invention can be formulated in liquid solutions, usefully in physiologically compatible buffers such as Hank's solution or Ringer's solution. In addition, the compounds may be formulated in solid form and redissolved or suspended immediately prior to use. Lyophilized forms are also included.

Systemic administration can be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the LcrV-related active compound. For buccal administration, the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the LcrV-related compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possibility of local noninvasive delivery of drugs over an extended period of time. This technology utilizes microspheres of precapillary size which can be injected via a coronary catheter into any selected part of the e.g. heart or other organs without causing inflammation or ischemia. The administered therapeutic is slowly released from these microspheres and taken up by surrounding tissue cells (e.g. endothelial cells).

In clinical settings, a therapeutic and gene delivery system for the LcrV protein or polypeptide-targeted therapeutic can be introduced into a patient by any of a number of methods, each of which is familiar in the art. For instance, a pharmaceutical preparation of the LcrV protein or polypeptide-targeted therapeutic can be introduced systemically, e.g., by intravenous injection.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### 5. EXAMPLES

The present invention is illustrated by the following examples. It is to be understood that the particular examples, materials, amounts, and procedures are to be interpreted broadly in accordance with the scope and spirit of the invention as set forth herein.

### 5.1 Example 1

### Passive Immunity to Yersiniae Mediated by Anti-Recombinant V Antigen and Protein A-V Antigen Fusion Peptide

Summary

In this example, LcrV of Y. pestis was cloned into protease-deficient Escherichia coli BL21. The resulting recombinant V antigen underwent marked degradation from the C-terminal end during purification, yielding major peptides of 36, 35, 34, and 32 to 29 kDa. Rabbit gamma globulin raised against this mixture of cleavage products provided significant protection against 10 minimum lethal doses of Y. pestis (P<0.01) and Y. pseudotuberculosis (P<0.02). To both stabilize V antigen and facilitate its purification, plasmid pPAV13 was constructed so as to encode a fusion of LcrV and the structural gene for protein A (i.e., all but the first 67 N-terminal amino acids of V antigen plus the signal sequence and immunoglobulin G-binding domains but not the cell wall-associated region of protein A). The resulting fusion peptide, termed PAV, could be purified to homogeneity in one step by immunoglobulin G affinity chromatography and was stable thereafter. Rabbit polyclonal gamma globulin directed against PAV provided excellent passive immunity against 10 minimum lethal doses of Y. pestis (P<0.005) and Y. pseudotuberculosis (P<0.005) but was ineffective against Y. enterocolitica. Protection failed after absorption with excess PAV, cloned whole V antigen, or a large (31.5-kDa) truncated derivative of the latter but was retained (P<0.005) upon similar absorption with a smaller (19.3-kDa) truncated variant, indicating that at least one protective epitope resides internally between amino acids 168 and 275.

Materials and Methods

Bacteria

E. coli K-12 XL1-Blue {recA1 endA1 gyrA96 thi-1 hsdR17 supE44 relA1 lac[F′ proAB lacIqZD.M15 Tn10 (Tetr)]} (Stratagene, La Jolla, Calif.) was used as a host for genetic engineering manipulations, and protease-deficient E. coli BL21 (F⁻ompT lon r_{B} ⁻m_{B} ⁻) (Novagen, Madison, Wis.) was utilized for expression of IcrGVH-yopBD under control of the tac promoter and production of PAV and truncated protein A. E. coli BL21(DE3) was used for biosynthesis of cloned gene products under control of the T7 promoter (Grodberg et al. (1988) J. Bacteriol. 170:1245-1253). The latter strain is lysogenic for DE3 which carries the T7 RNA polymerase gene under control of lacUV5 (Studier et al. (1986) J. Mol. Biol. 189:113-130).

Passively immunized mice were challenged with wild-type cells of Y. pseudotuberculosis PB 1/+ (Burrows et al. (1960) Br. J. Exp. Pathol. 41:38-44) or Y. enterocolitica WA of the highly virulent 0:8 serotype (Carter et al. (1980) Infect. Immun. 28:638-640). This purpose was accomplished with Y. pestis KIM by use of a nonpigmented mutant (Jacksou et al. (1956) Br. J. Exp. Pathol. 37:570-576; Surgalla et al. (1969) Appl. Microbiol. 18:834-837) known to lack a spontaneously deletable ca. 100-kb chromosomal fragment encoding functions of iron transport and storage (Fetherston et al. (1992) Mol. Microbiol. 6:2693-2704; Lucier et al. (1992) J. Bacteriol. 174:2078-2086); this isolate retained all other known chromosomally encoded virulence functions plus the Tox, Lcr, and Pst plasmids (Ferber et al. (1981) Immun. 31:839-841; Straley et al. (1982) Infect. Immun. 36:129-135). Mutants of this phenotype are virulent by intravenous injection (50% lethal dose, ca. 10 bacteria (Une and Brubaker (1984) J. Immunol. 133: 2226-2230) but not by peripheral routes of infection (50% lethal dose, >10⁷ bacteria (Brubaker et al. (1965) Science 149: 422-24).

Plasmids

The vector pKK223-3 containing the tac promoter (Pharmacia, Uppsala, Sweden) was used to express a portion of the IcrGVH-yopBD operon of Y. pestis 358 (Kutyrev et al. (1988) Anti-Plague Institute “Microbe” Press, Saratov, Russia) as described below. The vector pRIT5 (Pharmacia) encoding staphylococcal protein A was used for construction of gene fusions, as was the recombinant plasmid pBVP5 containing the IcrGVH-yopBD operon of Y. pseudotuberculosis (Motin et al. (1992) Microb. Pathog. 12:165-175). The latter was also used in preparation of deletion derivatives of lcrV yielding truncated derivatives of V antigen. The vector plasmid pBluescript SK+ (Stratagene) was introduced into E. coli BL21(DE3) for use in absorption of antiserum.

Cloning

Methods for preparation of plasmid DNA and its digestion with restriction enzymes, ligation, sequencing, and transformation into E. coli have been described previously (Sambrook et al. (1989) Molecular cloning: a laboratory manual, 2nd ed. Cold Spring Harbor Laboratory, Cold Spring Harbor, N.Y.). The 3.5-kb HindIII fragment of the Lcr plasmid of Y. pestis 358 was introduced into the expression vector pKK223-3. The resulting recombinant plasmid pKVE14 was then selected to ensure that the direction of transcription of the lcrGVH sequence corresponded to the action of the tac promoter.

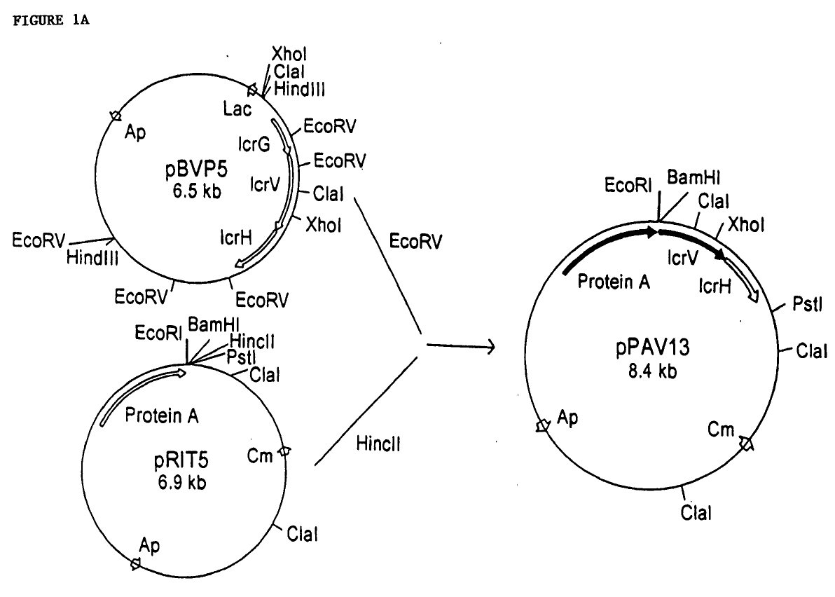

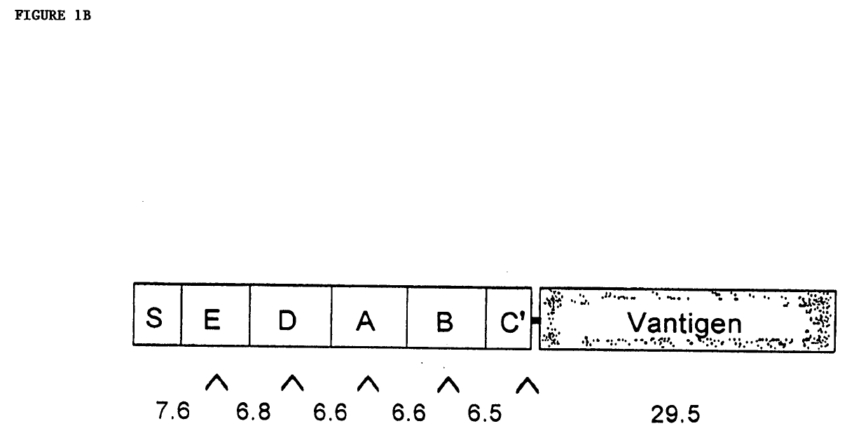

The schema used to construct pPAV13 containing a hybrid gene encoding a portion of protein A of Staphylococcus aureus and V antigen of Y. pseudotuberculosis is shown in FIG. 1A. The 1.5-kb EcoRV fragment of recombinant plasmid pBVP5 (Motin et al. (1992) Microb. Pathog. 12:165-175) was introduced into the vector pRIT5 encoding truncated protein A. The latter, either alone or fused with V antigen, maintained its signal sequence and most IgG-binding domains but lost the region mediating association with the bacterial cell surface (Nilsson et al. (1985) EMBO J. 4:1075-1080; Nilsson et al. (1985) Nucleic Acids Res. 13:1151 -1162) (see FIG. 1B). As a consequence of this fusion, lcrV lost 201 bp, causing deletion of the first 67 amino acide comprising the N-terminal portion of V antigen. The resulting PAV thus contained 305 N-terminal amino acids from protein A and 259 C-terminal amino acids from V antigen (FIG. 1B).

FIG. 1A shows the scheme of construction of recombinant plasmid pPA V13 encoding a staphylococcal protein A-V antigen fusion peptide termed PAV, and FIG. 1B shows the characterization of PAV protein—molecular masses (in kilodaltons) are indicated for each peptide arising after hydrolysis of the acid-labile Asp-Pro cleavage sites marked by arrowheads. Ap and Cm are locations of markers for resistance to ampicillin and chloramphenicol, respectively. Lac indicates the position of lacZ, which facilitates selection of recombinant plasmids in the vector pBluescript SK⁺. The genes lcrG, lcrV, and lcrH comprise a portion of the lcrGVH-yopBD operon of Y. pseudotuberculosis 995 (Motin et al. (1992) Microb. Pathog. 12:165-175), and the term protein A defines the location of the truncated protein A gene. The dark arrows in panel FIG. 1A represent the hybrid gene encoding PAV shown in FIG. 1B to consist of the signal sequence (S), IgG-binding domains (E to B), the defective domain C that has lost the ability to bind IgG, and truncated V antigen that has lost the first 67 amino acids of its N-terminal end. Molecular masses in kilodaltons are indicated for each peptide arising after hydrolysis of the acid-labile Asp-Pro cleavage sites marked by arrowheads (Uhlen et al. (1984) J. Biol. Chem. 259:1695-1702).

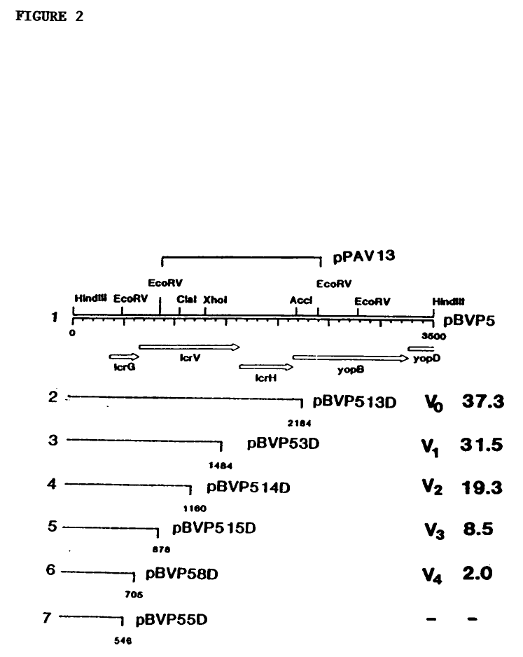

Deletion variants of lcrV were constructed by reducing the size of the 3.5-kb HindIII fragment of pBVP5 to 2.2 kb by cleavage of the AccI site downstream of lcrH. Prepared by this process, recombinant plasmid pBV513D contained the whole lcrGVH sequence under control of the T7 promoter (FIG. 2). Additional deletion variants were then prepared by digesting pBVP513D with exonuclease III followed by treatment with mung bean nuclease (Sambrook et al. (1989) Molecular cloning: a laboratory manual, 2nd ed. Cold Spring Harbor Laboratory, Cold Spring Harbor, N.Y.). The resulting set of plasmids retained the T7 promoter but lost progressively larger portions of the 3′ end of LcrV (which thus encoded a family of truncated V antigens that had lost correspondingly larger portions of the C-terminal end). Termini of each deletion variant were established by nucleotide sequencing and are shown in relationship to pPAV13 and pBVP5 with predicted molecular weights of the resulting truncated derivatives of V antigen (FIG. 2).

FIG. 2 shows the deletional variants constructed from pBVP5 consisting of the vector pBluescript SK⁺ HindIII fragment from the IcrGVH-yopBD operon of the Lcr plasmid of Y. pseudotuberculosis 995. The location of the EcoRV fragment used for construction of the fusion protein PAV is also indicated. Molecular masses (in kilodaltons) of truncated peptides of V antigen deduced from nucleotide sequences are shown on the right.

Termination of translation of these truncated derivatives of V antigen occurs in the vector portion of the established sequence of pBluescript SK+ (Stratagene) at bp 726 (frame 1) for pBVP53D, pBVP515D, and pBVP58D, thus increasing V₁, V₃, and V₄ by 9 amino acids; termination occurs at bp 776 (frame 3) for pBVP514D, thereby increasing V2 by 25 amino acids.

Purification of Recombinant V Antigen

Cells of E. coli BL21(pKVE14) were grown in fermentors as described previously (Brubaker et al. (1987) Microb. Pathog. 2:49-62) in medium consisting of 3% Sheffield NZ Amine, Type A (Kraft, Inc., Memphis, Tenn.), 0.5% NaCI, 1% lactose, and ampicillin (100 μg/ml) at 37° C. and harvested by centrifugation (10,000×g for 15 min) upon achieving an optical density at 620 nm of about 1.2. After disruption in a French pressure cell (SLM Instruments, Inc., Urbana, Ill.) and removal of insoluble matter by centrifugation (10,000×g for 30 min), V antigen was subjected to purification by an established procedure involving use of hydrophobic interaction chromatography with phenyl-Sepharose CL-4B (Pharmacia), ion-exchange chromatography with DEAE-cellulose (Whatman Inc., Clifton, N.J.), gel filtration with Sephacryl S-300F (Pharmacia), and calcium hydroxylapatite chromatography on Bio-Gel HTP (Bio-Rad, Richmond, Calif) (Brubaker et al. (1987) Microb. Pathog. 2:49-62). The original process was supplemented by a second chromatographic separation on DEAE-cellulose (linear gradient from 0 to 0.35 M NaCI) in order to remove high-molecular weight material unique to E. coli.

Preparation of Truncated Protein A and PAV

Cells of E. coli carrying pPAV 13 or pRIT5 were grown to late log phase at 37° C. in Luria broth containing ampicillin (50 μg/ml). Purification of these recombinant proteins was accomplished by affinity chromatography on IgG-Sepharose 6FF (Pharmacia) according to directions supplied by the manufacturer. Briefly, this process involved harvesting the organisms by centrifugation (10,000×g for 15 min) with resuspension at a ca. 10-fold increase in number in 0.01 M Tris-HCI, pH 8.0 (column buffer). Lysis was accomplished by initial addition of lysozyme (5 mg/m!) and then, after incubation for 1 h, further addition of Triton X-100 (0.1%), whereupon incubation was continued for 3 to 4 h. After clarification by centrifugation (10,000×g for 30 min), samples of 400 ml of the resulting soluble proteins were passed through a column (10 by 100 mm) containing a 10-ml packed volume of affinity resin that selectively bound truncated protein A or PAV. After addition and elution of 10 void volumes of column buffer to remove contaminating matter, the recombinant proteins were eluted with 0.2 M acetic acid (ca. pH 3.4), immediately frozen, and lyophilized. The resulting purified truncated protein A and PAV were then used directly for qualitative analysis and immunization.

Acid Hydrolysis of PAV

Purified PAV was treated with 70% formic acid for 20 hr. at 30° C. to cleave the four labile Asp-Pro peptide bonds within the truncated protein A domain (Dhlen et al. (1984) J. Biol. Chem. 259:1695-1702) and the additional site located at the junction with V antigen (Nilsson et al. (1985) EMBO J. 4:1075-1080) (FIG. 1B). After dialysis against column buffer, the partial hydrolysate was again passed through the IgG-Sepharose 6FF column as described above. In this case, the V antigen moiety plus fragments of the protein A domain lacking IgG-binding sites were immediately eluted whereas residual unhydrolyzed PAV remained bound to the affinity resin.

Preparation of Truncated Derivatives of V Antigen

Cells of E. coli BL21(DE3) transformed with pBVP5 and its deleted variants as well as the negative control pBluescript SK⁺ were grown in fermentors, harvested, and disrupted as described previously. After removal of insoluble material by centrifugation (10,000×g for 30 min), the resulting concentrated cell extract was subjected to molecular sieving on a column (5 cm by 1.5 m) of Sephadex G100 (Pharmacia) in 0.05 M CHES [2-(N-cyclohexylamino)-ethanesulfonic acid] buffer, pH 9.0. Samples containing V antigen or its truncated derivatives were identified by silver staining or immunoblotting, pooled, dialyzed against 0.05 M Tris-HCl, pH 8.0, and applied to a column (2.5 by 46 cm) of DEAE-cellulose equilibrated in the same buffer. All forms of V antigen became absorbed during this process, and, after passage of 2 void volumes of column buffer, they were eluted by batchwise application of the buffer containing 0.5 M NaCl. After dialysis, these concentrated samples were used directly to absorb IgG isolated from a known protective antiserum raised against PAV (described below) in order to determine the location of protective epitopes.

Preparation of Antisera

Rabbit polyclonal antiserum raised against V antigen purified from Y. pestis KIM, termed anti-native V antigen, has been characterized previously (Nakajima et al. (1993) Infect. Immun. 61:23-31) and was used as a positive immunological control. This antiserum plus the two rabbit polyclonal antisera directed against highly purified truncated protein A or PAV were obtained by use of Freund's adjuvant as described previously (Une et al. (1984) J. Immunol. 133:2226-2230). Less toxic TiterMax (Hunter's TiterMax no. R-1; CytRx Corp., Norcross, Ga.) was used to immunize rabbits against V antigen prepared from E. coli BL21(pKVEI4); this antiserum was termed anti-recombinant V antigen. Neither the latter nor antisera raised against the fusion proteins were absorbed with material from Lcr-bacteria, although highly purified gamma globulin was isolated from these reagents by the procedure used previously (Une et al. (1984) J. Immunol. 133:2226-2230).

Methods used for the preparation of monoclonal antibodies recognizing nonconformational epitopes of V antigen have been described previously (Brubaker (1991) Microbiol. Immunol. 12:127-133). As illustrated below, the first group of these antibodies reacted with an epitope present on the last 50 amino acids comprising the C-terminal part of V antigen (amino acids 276 to 326), as judged by ability to recognize V₀ (whole V antigen) but not V₁ or V₂ (monoclonal antibodies 3A4.1, 17A5.1, and 17A4.6). In contrast, monoclonal antibody 15A4.8 reacted with V₀ and V₁ but not V₂, indicating affinity for a shared internal epitope (amino acids 168 to 275).

Selective Absorption of Anti-PAV

Highly purified IgG prepared from anti-PAV was treated with excess PAV, V0, or its truncated derivative V₁ or V₂ according to an established protocol (Une et al. (1984) J. Immunol. 133:2226-2230). This process involved gentle aeration of the solution of IgG with an excess of a given antigen for 30 min. at 37° C. and then overnight incubation at 4° C. Precipitated material was removed by centrifugation (10,000×g for 30 min), and then the same process of absorption was repeated twice. Remaining free IgG and putative small IgG-V antigen complexes were then precipitated by 50% saturated (NH₄)₂S0₄, dialyzed against 0.05 M Tris-HCl, pH 7.8, and purified on a column (1.5 by 30 cm) of DEAE-cellulose by elution with the same buffer according to the method initially used for isolation of IgG. All forms of free V antigen or any IgG-V antigen complexes remaining after absorption were removed by this process. As a consequence, a set of highly specific antisera that progressively lost the ability to recognize the epitopes shared by truncated derivatives of V antigen were prepared.

Immunoblotting

Alkaline phosphatase conjugated with anti-rabbit or anti-mouse IgG (Sigma Chemical Co., St. Louis, Mo.) was usually used as a secondary antibody during immunoblotting by procedures described previously (Sample et al. (1987) Microb. Pathog. 3:239-248; Sample et al. (1987) Microb. Pathog. 2:443-453). These protocols were designed, in analysis of purified fractions, to maintain constant total activity of native V antigen (ca. 0.1 U per lane) and, in all other determinations, to maintain constant protein levels (7 to 10 μg per lane for cell lysates and 0.5 μg per lane for pure proteins). To prevent nonspecific reactions of monoclonal antibodies with truncated protein A and its derivatives, the nitrocellulose filter was first blocked with 5% fetal calf serum as usual and then incubated overnight with 1% normal human gamma globulin (Calbiochem, San Diego, Calif.); the latter (0.5%) was also added to solutions of primary and secondary antibodies (Lowenadler, B., et al. (1987) Gene 58:87-97). Fc-specific anti-mouse IgG (A-1418; Sigma) was used as a secondary antibody during immunoblotting of fusion proteins and their derivatives with monoclonal antibodies.

Passive Immunity

The ability of the antisera and preparations of purified IgG described above to provide passive immunity in Swiss Webster mice was determined by defined methods (Nakajima et al. (1993) Infect. Immun. 61:23-31; Une et al. (1984) J. Immunol. 133:2226-2230). This procedure involved intravenous injection of 10 minimum lethal doses of Y. pestis (102 bacteria), Y. pseudotuberculosis (102 bacteria), or Y. enterocolitica (103 bacteria) followed by intravenous administration of either 100 or 500 μg of purified IgG on postinfection days 1, 3, and 5.

Protein

Peptides were located in sodium dodecyl sulfate-polyacrylamide gel electrophoresis (SDS-PAGE) gels, prepared as previously described (Sample et al. (1987) Microb. Pathog. 3:239-248; Sample et al. (1987) Microb. Pathog. 2:443-453), by silver staining (Morrisey, J. H. (1981) Anal. Biochem. 117:307-310). Soluble protein was determined by the method of Lowry et al. (Lowry et al. (1951) J. Biol. Chem. 193:265-275). The statistical significance of the observed ability to provide passive immunity was determined by use of Fisher's exact probability test.

Results

Degradation of Recombinant V Antigen

Recombinant plasmid pKVE14 containing the lcrGVH-yopBD operon of Y. pestis under control of the tac promoter was transferred into protease-deficient E. coli BL21. After growth in fermentors, the bacteria were disrupted and the resulting extract was used to prepare nearly homogeneous recombinant V antigen (Table 1, below) by a method established for Ca²⁺-starved cells of Y. pestis (Brubaker et al. (1987) Microb. Pathog. 2:49-62). An additional step involving a second separation with DEAE-cellulose was necessary to eliminate major higher-molecular-weight proteins present in cytoplasm of E.

TABLE 1
Purification of recombinant V antigen from a cell extract of Escherichia coli BL21(pKVE14)

| | | Amt of | Total | Amt of V | Total V | | |
|---|---|---|---|---|---|---|---|
| | Vol | protein | protein | antigen | antigen | Sp | % |
| Preparation | (ml) | (mg/ml) | (nig) | (U/ml)^{a} | (U) | act^{b} | Recovery |
| Crude extract | 200 | 26 | 5,200 | 280 | 56,000 | 11 | 100 |
| Phenyl-Sepharose CL-4B | 220 | 1.6 | 350 | 140 | 30,800 | 88 | 55 |
| DEAE-cellulose | 40 | 1.5 | 60 | 170 | 6,800 | 113 | 12.1 |
| Sephacryl S-300SF | 24 | 0.7 | 17 | 140 | 3,360 | 200 | 6.0 |
| Ca hydroxylapatite | 35 | 0.25 | 8.8 | 50 | 1,750 | 200 | 3.1 |
| DEAE-cellulose (2nd separation) | 18 | 0.1 | 1.8 | 15 | 270 | 150 | 0.5 |

^{a}A unit of V antigen was defined as the reciprocal of the highest dilution capable of forming a visible precipitate against a standardized lot of rabbit polyclonal monospecific antiserum by diffusion in agar under conditions described previously (Brubaker et al. (1987) Microb. Pathog. 2: 49-62; Lawton et al. (1963) J. Immunol. 91: 179-184).
^{b}Specific activity is in units per milligram of protein.

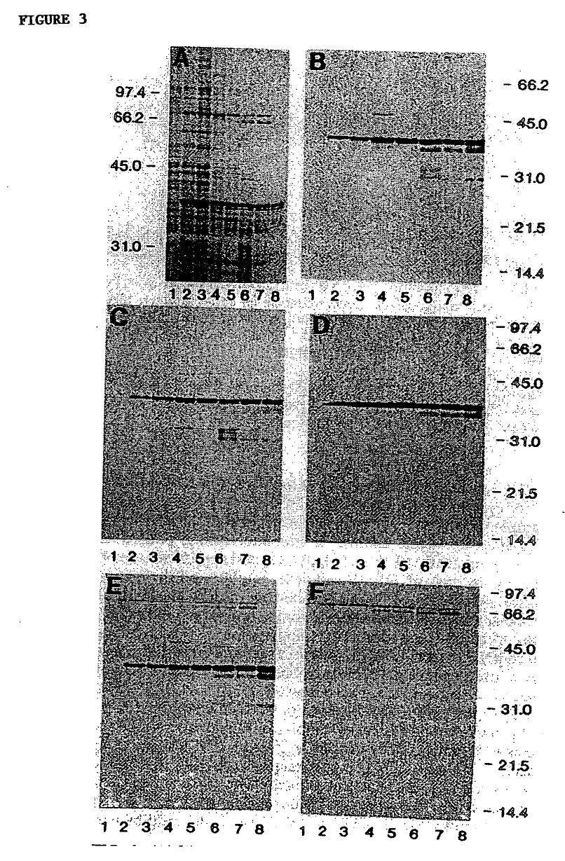

The initial specific activity of recombinant V antigen was almost fivefold greater than that obtained from Y. pestis starved of Ca²⁺ (Brubaker et al. (1987) Microb. Pathog. 2:49-62). Nevertheless, significant loss of precipitin activity occurred during every step of purification (Table 1). This phenomenon, as judged by inspection of a silver-stained lane gel (FIG. 3A), reflected gradual loss of the native 37-kDa form of V antigen with emergence of ca. 36- and 32-kDa and possibly smaller degradation forms. Analysis by immunoblotting was undertaken to prove that these new peptides shared epitopes with and thus arose from native V antigen. Use of rabbit polyclonal anti-native V antigen (FIG. 3B) or mouse monoclonal antibody 15A4.8, directed against a centrally located epitope (FIG. 3C), demonstrated emergence of new ca. 36-, 35-, and 34-kDa products early during the course of purification, with later appearance of a series of smaller fragments ranging from 32 to 29 kDa. The latter were not recognized by mouse monoclonal antibody 3A4.1 directed against an epitope located near the C-terminal end of native V antigen (FIG. 3D). These findings indicate that recombinant V antigen produced in the cytoplasmic background of E. coli BL21 undergoes evident spontaneous hydrolysis in a manner similar to that observed for native V antigen expressed in Y. pestis (Brubaker et al. (1987) Microb. Pathog. 2:49-62) and that this process of degradation is initiated at the C-terminal end of the peptide.

FIG. 3A shows a silver-stained extended SDS-12.5% PAGE gel of whole cells of E. coli BL21 containing the vector plasmid pK223-3 (lane 1) or recombinant plasmid pKVE14 (lane 2). Whole cells of the latter were disrupted and centrifuged to prepare a cell extract (lane 3) that was fractionated by chromatography on phenyl-Sepharose CL-4B (lane 4), DEAE-cellulose (lane 5), Sephacryl S-300SF (lane 6), calcium hydroxyapatite (lane 7), and second-passage DEAE-cellulose (lane 8); V antigen appears as a major peptide of 37 kDa in lanes 2 through 8. The same samples were immunoblotted against rabbit polyclonal antinative V antigen (B), mouse monoclonal anti-V antigen 15A4.8 (C), mouse monoclonal anti-V antigen 3A4.1 (D), rabbit polyclonal anti-PAV (E), and rabbit polyclonal anti-truncated staphylococcal protein A (F). Numbers on the left and right indicate molecular masses in kilodaltons.

Characterization of Truncated Protein A and PAV

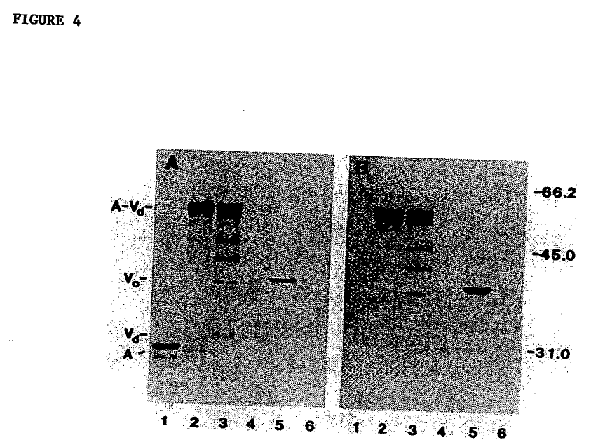

Additional constructions encoding a portion of the structural gene for staphylococcal protein A alone or this gene fused with lcrV (FIG. 1A) were found, after transformation into E. coli BL21, to promote significant synthesis of truncated protein A and PAV, respectively, as judged by the intensity and specificity of reactions observed in the immunoblots described below. These two peptides were purified to homogeneity in one step by affinity chromatography and then analyzed by immunoblotting. Polyclonal anti-native V antigen reacted nonspecifically with truncated protein A (FIG. 4A, lane 1) and both specifically and nonspecifically with PAV (FIG. 4A, lane 2). Proof that polyclonal anti-V antigen specifically recognized PAV and its derivatives (shown in lanes 2, 3, and 4 of FIG. 4A) was obtained by blocking the protein A domain with human gamma globulin and then immunoblotting with a monoclonal anti-V antigen. This process prevented visualization of truncated protein A (FIG. 4B, lane 1). Accordingly, all of the remaining bands visible in FIG. 4B reflect the occurrence of a specific interaction with an epitope of V antigen. Multiple bands appearing in samples of both truncated protein A (FIG. 4A, lane 1) and PAV (FIG. 4A, lane 2) represent accumulation of native and degraded forms of the protein A domain (FIG. 1B) within the periplasm of E. coli BL21 (Gaudecha et al. (1992) Gene 122:361-365). To prove that the linked V antigen domain was stable, a sample of PAV was hydrolyzed with 70% formic acid to cleave acid-labile Asp-Pro linkers (FIG. 1B), neutralized, and then applied to the affinity column. Essentially pure truncated V antigen (Vd) emerged immediately (FIG. 4B, lane 4). The absence of multiple bands in this sample indicates that the V antigen domain within PAV (FIG. 1B) did not undergo degradation during purification as was described above to occur with free V antigen.

FIG. 4A shows immunoblots prepared with polyclonal anti-native V antigen, and FIG. 4B shows immunoblots prepared with mouse monoclonal anti-V antigen 17A5.1 directed against truncated protein A (lanes 1), PAV (lanes 2), PAV partially hydrolyzed by formic acid (lanes 3), PAV partially hydrolyzed by formic acid and then passed through the IgG-Sepharose 6FF column (lanes 4), whole Ca²⁺-starved Lcr⁺ cells of Y. pestis KIM (lanes 5), and whole Ca²⁺-starved Lcr⁻ cells of Y. pestis KIM (lanes 6); A-V_{d} V₀, V_{d} and A indicate the positions of PAY, native V antigen (37.3 kDa), truncated V antigen (29.5 kDa), and truncated protein A, respectively. Human gamma globulin was used to block nonspecific reactions of monoclonal antibodies against IgG-binding domains of protein A (Lowenadler et al. (1987) Gene 58:87-97). Numbers on the right indicate molecular masses in kilodaltons.

The number of total units of PAV purified by affinity chromatography was always identical to that present in the crude extract applied to the column. No significant loss of purified PAV occurred during storage in 0.01 M Tris-HCl (pH 7.8) for 1 week at 4° C.

Characterization of Antisera Raised Against Recombinant V Antigens

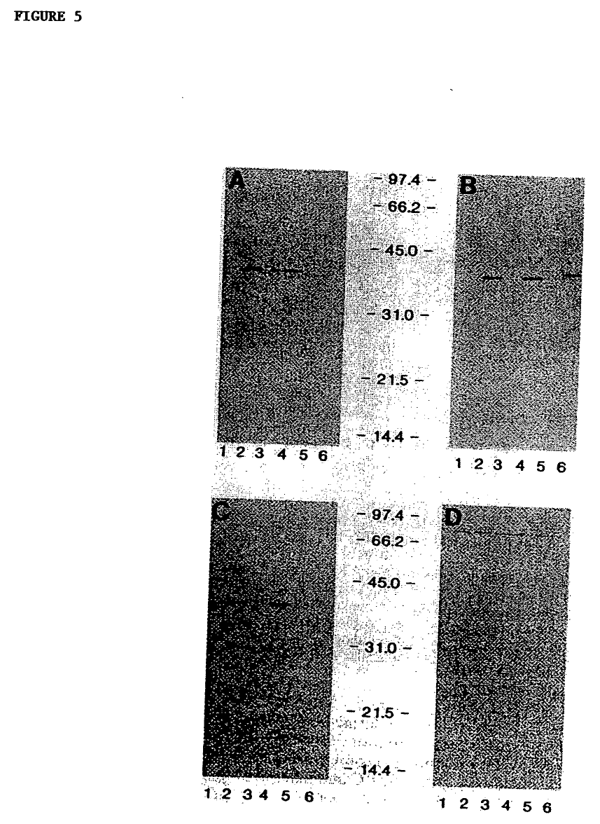

Preparations of homogeneous gamma globulin were isolated from unabsorbed rabbit antisera raised against purified recombinant V antigen, PAV, and truncated protein A. The specific reaction obtained by immunoblotting Lcr⁺ and Lcr⁻ yersiniae containing the native 37-kDa V antigen of Y. pestis and Y. pseudotuberculosis and the 42-kDa V antigen of Y. enterocolitica (Mulder et al. (1989) Infect. Immun. 57:2534-2541) with control absorbed anti-native V antigen (FIG. 5A) was duplicated with anti-recombinant V antigen (FIG. 5B) and anti-PAV (FIG. 5C) but not with anti-truncated protein A (FIG. 5D). However, normal serum (data not illustrated), as well as the three unabsorbed antisera, also recognized unknown high-molecular-mass antigens (ca. 70 kDa) shared by Lcr⁺ and Lcr⁻ organisms. Anti-PAV (FIG. 3E) but not anti-truncated protein A (FIG. 3F) reacted with the same degradation products of recombinant V antigen that were identified upon assay with anti-native V antigen (FIG. 3B) and monoclonal antibody 15A4.8 (FIG. 3C).

FIG. 5A shows immunoblots prepared with absorbed rabbit polyclonal anti-native V antigen purified from Y. pestis KIM, FIG. 5B shows immunoblots prepared with anti-recombinant V antigen, FIG. 5C shows immunoblots prepared with anti-PAV, FIG. 5D shows immunoblots prepared with anti-truncated protein A, each directed against Ca²⁺-starved whole cells of Lcr⁻ Y. pestis KIM (lanes 1), Lcr⁻ Y. pestis KIM (lanes 2), Lcr⁻ Y. pseudotuberculosis PB 1 (lanes 3), Lcr⁺ Y. pseudotuberculosis PB 1 (lanes 4), Lcr⁻ Y. enterocolitica WA (lanes 5), and Lcr⁺ Y. enterocolitica WA (lanes 6). Numbers down the middle indicate molecular masses in kilodaltons.

Passive Immunity Mediated by Anti-Recombinant V Antigens

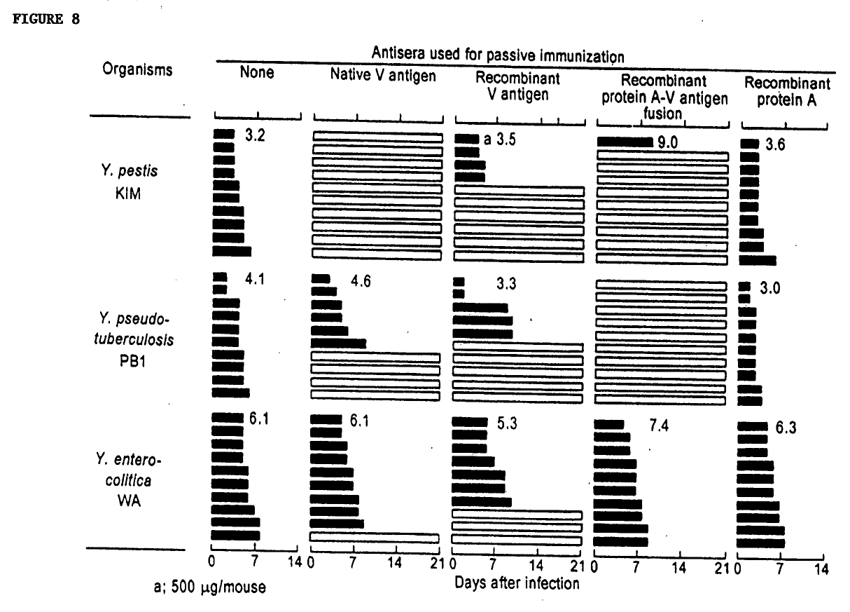

As anticipated from prior work (Une et al. (1984) J. Immunol. 133:2226-2230), control anti-native V antigen provided significant passive immunity against intravenous challenge with 10 minimum lethal doses of Lcr⁺ cells of Y. pestis (P<0.005) and Y. pseudotuberculosis (P<0.05) but not Y. enterocolitica (Table 2). Anti-recombinant V antigen provided similar protection against challenge with Y. pestis (P<0.01) and Y. pseudotuberculosis (P<0.02), as did anti-PAV (P<0.01 for Y. pestis and P<0.005 for Y. pseudotuberculosis), whereas treatment with anti-truncated protein A was without effect. FIG. 8 is a diagrammatic representation summarizing the ability of IgG isolated from normal rabbit serum, as well as antisera raised against various V antigen antisera preparations indicated and a protein A control, to provide passive immunity against intravenous challenge with the various indicated Yersinia species and strains.

TABLE 2
Ability of IgG isolated from normal rabbit serum and from antisera raised against native V
antigen, recombinant V antigen, PAV, and truncated protein A to provide passive immunity
against intravenous challenge with 10 minimum lethal doses of Lcr⁺ yersiniae

| | | | | | | | | | | | No. | |
|---|---|---|---|---|---|---|---|---|---|---|---|---|
| | | No. of mice surviving on | | | | | | | | | dead/ | |
| | | day after infection: | | | | | | | | | total | |
| Challenge Organism | Source of IgG^{a} | 1 | 4 | 5 | 6 | 7 | 8 | 9 | 10 | 21 | no. | pb |
| Y. pestis KIM | Normal Serum | 10 | 6 | 4 | 1 | 0 | | | | | 10/10 | |
| | Anti-native V antigen | 10 | 10 | 10 | 10 | 10 | 10 | 10 | 10 | 10 | 0/10 | <0.005 |
| | Anti-recombinant V antigen | 10 | 8 | 6 | 6 | 6 | 6 | 6 | 6 | 6 | 4/10 | <0.01 |
| | Anti-PAV | 10 | 10 | 10 | 10 | 10 | 10 | 9 | 9 | 9 | 1/10 | <0.01 |
| | Anti-truncated protein A | 10 | 3 | 1 | 0 | | | | | | 10/10 | NS |
| Y. pseudotuberculosis | Normal Serum | 10 | 8 | 4 | 1 | 0 | | | | | 10/10 | |
| PBI | Anti-native V antigen | 10 | 9 | 8 | 7 | 7 | 7 | 4 | 4 | 4 | 6/10 | <0.05 |
| | Anti-recombinant V antigen | 10 | 8 | 8 | 8 | 8 | 8 | 7 | 5 | 5 | 5/10 | <0.02 |
| | Anti-PAV | 10 | 10 | 10 | 10 | 10 | 10 | 10 | 10 | 10 | 0/10 | <0.005 |
| | Anti-truncated protein A | 10 | 2 | 0 | | | | | | | 10/10 | NS |
| Y. enteroocolitica WA | Normal Serum | 10 | 10 | 10 | 6 | 3 | 2 | 0 | | | 10/10 | |
| | Anti-native V antigen | 10 | 10 | 10 | 8 | 6 | 4 | 2 | 1 | 1 | 9/10 | NS |
| | Anti-recombinant V antigen | 10 | 10 | 10 | 10 | 7 | 7 | 4 | 3 | 3 | 7/10 | NS |
| | Anti-PAV | 10 | 10 | 10 | 9 | 7 | 4 | 2 | 0 | | 10/10 | NS |
| | Anti-truncated protein A | 10 | 10 | 10 | 7 | 4 | 2 | 0 | | | 10/10 | NS |

^{a}Mice received 100 μg of IgG in 0.1 ml of 0.033 M KHP0₄⁻, pH 7.0, by intravenous injection on postinfection days 1, 3, and 5 (except for anti-recombinant V antigen, of which 500 μg was injected).
^{b}Determined by Fisher's exact probability test; NS, not significant.

Truncated V Antigens

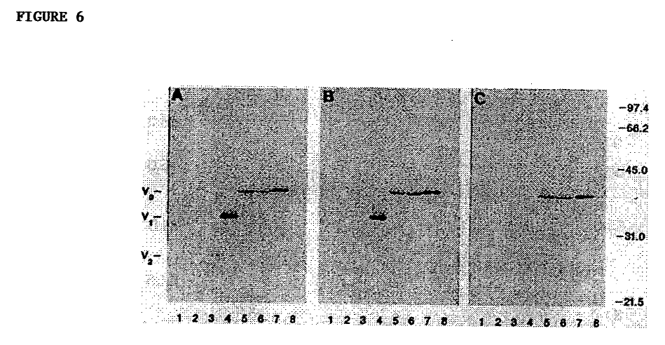

A series of recombinant plasmids containing deletions of increasing size in lcrV of Y. pseudotuberculosis was constructed; predicted molecular weights of the resulting entire V antigen (V₀) and its truncated derivatives (V₁ to V₄) are given in FIG. 2. The expression and actual sizes of these peptides were determined by immunoblotting. V₀ and V₁ exhibited strong reactions against anti-native V antigen, which barely detected V₂ (FIG. 6A); no interaction with V₃ or V₄ was observed (data not illustrated). Monoclonal antibody 15A4.8 failed to react with V₂ but recognized both V₀ and V₁ (FIG. 6B), indicating that its target epitope resides internally within the primary structure shared between the C-terminal ends of V₂ and V₁ (amino acids 168 to 275). In contrast, monoclonal antibody 17AS. 1 recognized only V₀ (FIG. 6C), demonstrating that its target epitope resides within the amino acid sequence located between the C-terminal ends of V₁ and V₀ (amino acids 276 to 326). Identical results were obtained with mouse monoclonal antibodies 3A4.1 and 17A4.6. These results demonstrate that V₀ and V₁ were produced in abundance. Significant levels of less antigenic V₂ (as opposed to V₃ and V₄) were also expressed, as judged by the ability of this peptide to selectively remove specific antibodies from anti-PAV (described below). These results suggest that polyclonal antisera directed against V antigen primarily recognize epitopes located near the C-terminal rather than the N-terminal end of the peptide.

FIG. 6 shows immunoblots prepared with absorbed rabbit polyclonal anti-native V antigen (FIG. 6A), mouse monoclonal anti-V antigen 15A4.8 (FIG. 6B), and mouse monoclonal anti-V antigen 17A5.1 (FIG. 6C) against extracts of E. coli BL21(DE3)/pBluescript SK+ (lanes 1), E. coli BL21(DE3)/pBVP515D (lanes 2), E. coli BL21(DE3)/pBVP514D (lanes 3), E. coli BL21(DE3)/pBVP53D (lanes 4), E. coli BL21(DE3)/pBVP513D (lanes 5), E. coli BL21(DE3)/pBVP5 (lanes 6), Lcr⁺ Y. pestis KIM (lanes 7), and Lcr⁻ Y. pestis KIM (lanes 8). Numbers on the right indicate molecular masses in kilodaltons.

Selective Absorption of Anti-PAV

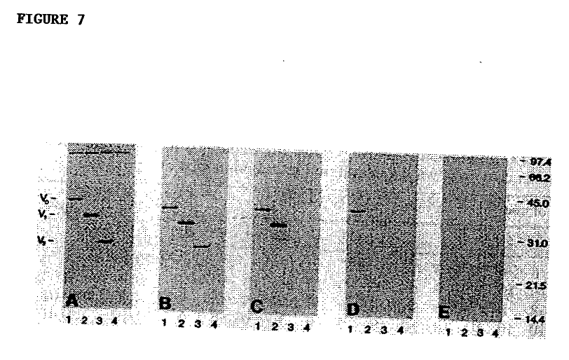

Cells of E. coli BL21 (DE3) carrying plasmids pBVP513D, pBVP53D, and pBVP514D encoding V₀, V₁, and V₂, respectively, were induced in fermentors, and, after disruption, the resulting cytoplasmic extracts were subjected to a process involving separation by size and net charge that resulted in isolation of sufficient concentrations of the three peptides to permit selective absorption of anti-PAY. As shown in FIG. 7A, unabsorbed anti-P A V recognized V₀, V₁, and V₂, as well as the high-molecular-weight antigens noted previously (FIGS. 3E and F and 5C and D). Antibodies to the latter could be removed by absorption with excess product obtained by parallel purification from extracts of control cells of E. coli BL21(DE3) containing the vector pBluescript SK⁺ alone (FIG. 7B). Similar absorption with excess products prepared from isolates of E. coli BL21(DE3) carrying pBVP514D, pBVP53D, and pBVP5 progressively removed antibodies directed specifically against V₂ (FIG. 7C), V₁ (FIG. 7D), and V₀ (FIG. 7E), respectively.

FIG. 7 shows immunoblots prepared with rabbit polyclonal anti-PAV without absorption (FIG. 7A), and after exhaustive absorption with preparations of E. coli BL21(DE3) transformed with pBluescript SK⁺, pVBP514D, pBVP53D, or pBVP5 containing shared proteins alone (FIG. 7B), shared proteins plus excess V₂ (FIG. 7C), shared proteins plus excess V₁ (FIG. 7D), or shared proteins plus excess V₀ (FIG. 7E), respectively. Extracts of E. coli BL21(DE3)/pBVP5 containing V₀ (lanes 1), E. coli BL21(DE3)/pBVP53D containing V₁, (lanes 2), E. coli BL21(DE3)/pBVP514D containing V₂ (lanes 3), and E. coli BL21 (DE3)/pBluescript SK⁺ (vector plasmid control) (lanes 4) are shown. Numbers on the right indicate molecular masses in kilodaltons.

Passive Immunity Mediated by Anti-PAY Absorbed with Excess Truncated V Antigens

IgG purified from anti-PAV, absorbed as described above with excess V₀ or its truncated derivatives, was used to assay for ability to provide passive immunity against 10 minimal lethal doses of Lcr⁺ cells of Y. pestis or Y. pseudotuberculosis. In this determination (Table 3), lethality to untreated mice was absolute and occurred rapidly in a pattern similar to those observed for controls treated with purified normal IgG or IgG isolated from anti-truncated protein A. In contrast, all mice survived following administration of IgG from unabsorbed anti-PAV (P<0.005) or that from anti-PAV absorbed with excess preparation of vector (P<0.005) or V₂ (P<0.005). Similar absorption of IgG from anti-PAV with excess V₁, V₀, or PAV itself rendered the antiserum ineffective. This finding provides formal proof that V antigen per se is a protective antigen and indicates that at least one epitope responsible for immunity resides internally within the primary structure spanning the C-terminal end points of V₂ and V₁ (amino acids 168 to 275).

TABLE 3
Ability of IgG isolated from rabbit polyclonal anti-PAV to provide passive immunity against
intravenous challenge with 10 minimum lethal doses of Y. pestis KIM following absorption with
excess PAV, V antigen, and truncated derivatives V₁and V₂

| | | | | | | | | | | | | No. | |
|---|---|---|---|---|---|---|---|---|---|---|---|---|---|
| | Product used | No. of mice surviving on day after infection: | | | | | | | | | | dead/ | |
| Source of IgG^{a} | for absorption | 0 | 1 | 2 | 3 | 4 | 5 | 6 | 7 | 14 | 21 | total no. | pb |

Y. pestis KIM

| | | | | | | | | | | | | | |
|---|---|---|---|---|---|---|---|---|---|---|---|---|---|
| None | None | 5 | 5 | 5 | 5 | 3 | 3 | 0 | | | | 5/5 | |
| Normal serum | None | 5 | 5 | 5 | 5 | 4 | 3 | 0 | | | | 5/5 | NS |
| Anti-truncated protein A | None | 5 | 5 | 5 | 5 | 4 | 3 | 1 | 0 | 0 | 0 | 5/5 | NS |
| Anti-PAV | None | 5 | 5 | 5 | 5 | 5 | 5 | 5 | 5 | 5 | 5 | 0/5 | <0.005 |
| Anti-PAV | Vector | 5 | 5 | 5 | 5 | 5 | 5 | 5 | 5 | 5 | 5 | 0/5 | <0.005 |
| Anti-PAV | V₂ | 5 | 5 | 5 | 5 | 5 | 5 | 5 | 5 | 5 | 5 | 0/5 | <0.005 |
| Anti-PAV | V₁ | 5 | 5 | 5 | 5 | 5 | 4 | 3 | 0 | | 0 | 5/5 | NS |
| Anti-PAV | V₀ | 5 | 5 | 5 | 5 | 4 | 3 | 1 | 0 | | 0 | 5/5 | NS |
| Anti-PAV | PAV | 5 | 5 | 5 | 5 | 4 | 3 | 0 | | | | 5/5 | NS |

Y. pseudotuberculosis PB1

| | | | | | | | | | | | | | |
|---|---|---|---|---|---|---|---|---|---|---|---|---|---|
| None | None | 5 | 5 | 5 | 5 | 4 | 3 | 0 | | | | 5/5 | |
| Normal serum | None | 5 | 5 | 5 | 5 | 4 | 3 | 0 | | | | 5/5 | NS |
| Anti-truncated protein A | None | 5 | 5 | 5 | 5 | 3 | 4 | 1 | 0 | | 0 | 5/5 | NS |
| Anti-PAV | None | 5 | 5 | 5 | 5 | 5 | 5 | 5 | 5 | 5 | 5 | 0/5 | <0.005 |
| Anti-PAV | Vector | 5 | 5 | 5 | 5 | 5 | 5 | 5 | 5 | 5 | 5 | 0/5 | <0.005 |
| Anti-PAV | V₂ | 5 | 5 | 5 | 5 | 5 | 5 | 5 | 5 | 5 | 5 | 0/5 | <0.005 |
| Anti-PAV | V₁ | 5 | 5 | 5 | 5 | 2 | 0 | 0 | | | | 5/5 | NS |
| Anti-PAV | V₀ | 5 | 5 | 5 | 5 | 4 | 2 | 1 | 0 | | 0 | 5/5 | NS |
| Anti-PAV | PAV | 5 | 5 | 5 | 5 | 4 | 3 | 0 | | | | 5/5 | NS |

^{a}Mice received 100 μg in 0.1 mg of 0.033 M KHP0₄⁻, pH 7.0, by intravenous injection on postinfection days 1, 3, and 5.
^{b}Determined by Fisher's exact probability test; NS, not significant.

Experimental evidence supporting the assumption that anti-V antigen provides immunity against plague was initially limited to the findings that active immunization with V antigen-rich fractions (Burrows et al. (1958) Br. J. Exp. Pathol. 39:278-291) or passive immunization with antisera raised against such fractions (Lawton et al. (1963) J. Immunol. 91:179-184) promoted protection against experimental disease. The observation that cloned V antigen expressed in the protease-deficient background of E. coli BL21, like native V antigen purified from Y. pestis, underwent marked degradation during preparation is consistent with either the occurrence of autocatalytic hydrolysis or conversion to a steric form after partial purification, resulting in vulnerability to the inherent stresses of physical isolation (or to distinct contaminating proteases).

A stable fusion protein PAV was developed with increase specificity and lower degradation rates. This fusion protein could be isolated in one step at high yield in a homogeneous state. Rabbit polyclonal anti-PAV, like anti-native or anti-recombinant V antigen, was effective in providing passive immunity against Y. pestis and Y. pseudotuberculosis. This finding emphasizes that expression of protection did not require the presence of antibody against LcrG (linked upstream) or N-terminal epitopes of V antigen, because these sequences were absent in PAV used for immunization. The decision to sacrifice the N-terminal rather than the C-terminal end of V antigen to construct a fusion with protein A was based in part on the assumption that this region, like the N terminal of Yops noted above, is involved in an exit reaction rather than catalysis of some biological activity directed against the host. Absorption of anti-PAV with excess truncated derivatives of V antigen lacking LcrH (linked downstream) provided further information about the location of protective epitopes. In these experiments, sufficient PAV, V₀, V₁ V₂ or a parallel preparation from cells carrying the plasmid vector alone was added to anti-PAV to selectively remove all corresponding antibodies detectable by immunoblotting. Assay of the resulting antisera showed that absorption with excess PAV, V₀, or V₁ but not V₂ or the vector control removed all protective antibodies. This observation suggests that at least one protective epitope is located between the C-terminal points of V₂ and V₁ (amino acids 168 to 275). Demonstration that a monospecific antiserum loses its ability to provide passive immunity upon absorption with an excess of its opposing antigen provides formal proof that antigen is protective. This criterion was met for V antigen in this study.

Full active immunization of mice with PAV may result in an equivalent increase in 50% lethal dose following challenge with Lcr⁺ cells of Y. pestis. In contrast, anti-V antigen was clearly ineffective in providing passive immunity against infection by highly invasive serotype 0:8 cells of Y. enterocolitica.

### 5.2 Example 2

### Binding of Y. pestis LcrV at Dual Sites to TLR-2 and IFN-γReceptor

In this example, dual binding sites of Yersiniae LcrV that mediate interaction with host TLR-2 and IFN-γ receptors were identified. As discussed above, LcrV of Yersinia pestis regulates, targets, and mediates type III translocation of cytotoxins into host cells and binds to TLR-2 thereby upregulating anti-inflammatory IL-10; and protective anti-LcrV neutralizes at least one of these functions. This example shows that native LcrV binds TLR-2 at an internal site before associating with the human TLR-2 receptor of monocytes causing prompt upregulation of IL-10 and inhibition of the oxidative burst. These responses were initiated by evident dual binding sites located at the N-terminus (amino acids 32-35) and internally (amino acids 203-206) comprising adjacent glutamic acid residues flanked by hydrophobic amino acids. High affinity attachment as evidenced by Scatchard analysis (characterized by dissociation constants of −10⁻¹⁰) occurred with adjoining arginine residues of human TLR-2 and the C-terminus of bound IFN-γ. Association of LcrV with TLR-2 and receptor-bound IFN-γ was not cooperative and only the latter site appears to function in native LcrV. Both binding sites are removed by five amino acids from aspartic acid-lysine-asparagine motifs. The interaction with the IFN-γ receptor is CD14-independent and caused prompt upregulation of IL-10 in human monocytes, concomitant downregulation of LPS-induced amplification of TNF-α, and inhibition of the oxidative burst in human neutrophils.

These dual binding sites of LcrV are targets for protective immunization as they facilitate pathogenesis of plague by serving as a surface adhesin promoting close bacterium-host cell contact necessary for Yop translocation. Accordingly, antibodies directed against these binding sites block an early stage of pathogenic Yersinia infection. These results demonstrate that LcrV possesses two non-cooperative binding domains capable of recognizing both free TLR-2 and IFN-γ bound to its receptor (IFN-γR-IFN-γ) and that the site unique to amino acids 168-275 functions within the native molecule. In addition, we demonstrate that LcrV utilizes both domains to upregulate IL-10, downregulate LPS-induced TNF-α, and prevent oxidative killing by neutrophils.

Materials and Methods

Recombinant Proteins

LcrV was produced using IcrV present within the IcrGVH-yopBD operon of pCD1 from Y. pestis strain KIM 10 (Finegold et al. (1968) Am. J. Pathol., 53:99-114) prepared by amplification with PCR using sites for EcoRI and BarrHI, and then inserted into the vector pRSET A (Invitrogen) opened with BamHI plus EcoRI. This construct, expressed in E. coli BL21(DE3), encodes N-terminal hexahistidine, an enterokinase cleavage site, and then LcrV in its entirety. Similar preparation of E. coli BL21(DE3) transformed with pVHB62 encoding LcrV₆₈₋₃₂₆ has been described (Motin et al. (1996) Infect. Immun., 64:4313-4318). LcrV and LcrV₆₈₋₃₂₆ encoded by these constructs were induced by IPTG, purified to near-homogeneity by Ni-affinity chromatography, and then freed of hexahistidine by treatment with enterokinase (Motin et al. (1996) Infect. Immun., 64:4313-4318). Recombinant human IFN-γ with antiviral activity of 1.5×10⁷ U/mg was supplied by Dr. V. Fedyukin (JSC “ImmunoPharm”, Obolensk, Russia). Highly purified human TNF-α, IFN-α₂ and EGF were purchased from PeproTech (UK).

FIG. 9A shows the primary amino acid sequence of LcrV from Y. pestis KIM (18), where the amino acids of the primary construct (LcrV68-326) initially used to formally demonstrate ability to raise protective anti-bodies and amplify IL-10 are in non-italicized font. The location of the synthetic peptides VLEELVQLVKDKNIDISIKY (LcrV31-50) (SEQ ID NO: ______) and LMDKNLYGYTDEEIFKAS (LcrV193-210) used in this study are in bold and their putative binding sites containing adjacent glutamic acid residues are underlined; note the presence of DKN motifs removed from each binding site by five amino acids.

Synthetic Peptides

The peptides VLEELVQLVKDKNIDISIKY (LcrV₃₁₋₅₀) (SEQ ID NO: ______) and LMDKNLYGYTDEEIFKAS (LcrV₁₉₃₋₂₁₀) comprise portions of LcrV and were synthesized with a solid-phase model 9500 peptide synthesizer (Biosearch, USA). The relationship of these peptides with previously established protective N-terminal truncated derivative (LcrV₆₈₋₃₂₆) (Motin et al. (1994) Infect. Immun., 62:4192-4201) and whole LcrV is shown in FIG. 9A. This apparatus was also used to synthesize the peptides FNPSESDVVSELGKVETVTIRRLHIPQ (SEQ ID NO: ______) and SQMLFRGRRASQ (SEQ ID NO: ______) from the C-terminus of IFN-γ and the extracellular domain of mouse TLR-2, respectively.

Excherichia coli LPS was purchased from Calbiochem (La Jolla, Calif.). Also used in experiments were LymphoSep™-lymphocyte separation medium, fetal calf serum (FCS), and RPMI1640 medium (ICN Biomedicals, Inc) Zymosan A, Luminol, Dulbecco's modified Eagle's medium (DMEM), 1,3,4,6-tetrachloro-3α,6α-DI-phenylglycouril (lodo-Gen), Triton X-100, propidium iodide, colorless Hank's Balanced Salt Solution (HBSS), and HEPES (Sigma, St. Lousi, Mo.); Sephadex G-25 (LκB, Sweden); Ficoll Hypaque (Pharmacia, Sweden), and Na¹²⁵I (Amersham, UK).

Cell Lines

Human monocytic leukemia cell line U937 and human thymic epithelial cell line VTEC2.H2 transformed with SV₄₀ virus were obtained from the Institute of Carcinogenesis (Moscow, Russia) and Institute of Immunology (Moscow, Russia), respectively. The former was maintained in suspension culture (2-9×10⁵ cells/ml) in RPMI 1640 medium supplemented with 10% (v/v) fetal calf serum, HEPES buffer (15.0 mM), L-glutamine (2.0 mM), penicillin (100 U/ml), and streptomycin (100 U/ml). VTEC2.H2 cells were cultured as a suspension (2 to 7×10⁵ cells/ml) in the same medium.

Cell Preparations

Normal human thymocytes were obtained from children who underwent thymectomy during cardiac surgery (Cardiocenter, Moscow, Russia). A suspension of individual cells was obtained upon thymus disintegration and subsequent purification using a Ficoll Hypaque gradient. The resulting free thymocytes were then washed three times in RPMI 1640 medium containing 2% fetal calf serum. Viability was 96 to 98% as judged by methylene blue staining (Hobbs et al. (1993) J. Imrnunol., 150:3602-3614). Monolayers of thymic epithelial cells were obtained as a consequence of 3-4 passages of adhered thymocytes incubated with human recombinant epidermal growth factor (20 ng/ml) in DMEM and RPMI 1640 media. At least 80% of these cells contained keratin.

Human neutrophils from blood purchased from the Blood Transfusion Station (Chekov, Moscow region, Russia) were isolated by centrifugation for 40 min. on a 400 g double density Ficoll Hypaque gradient (upper and lower layer densities of 1.077 and 1.119 g/ml, respectively). The resulting neutrophils, used for analysis of ability to generate reactive forms of oxygen, were recovered from the lower ring above the 1.119 density solution and found to be 96-98% pure. Human monocytes were isolated from blood by centrifugation over Lympho Separation Medium. Interface cells in RPMI 1640 medium with 10% fetal calf serum (10⁶ per ml) were placed in wells for monolayer formation and after 1 hr. cells unattached to the plastic surface were removed with replacement of volume with the same medium. The resulting monolayer contained over 98% viable monocytes after culture for 24 hr. at 37° C. with 5% CO₂ and was directly used to characterize expression of cytokines.

Radiolabeling

¹²⁵I-LcrV and derivatives (all 0.09 mCu/μg) as well as IFN-γ (0.1 mCu/μg) and IFN-α (0.1 mCu/μg) were prepared by iodination with Iodo-Gen (Sigma) and Na¹²⁵I and then separated by chromatography on Sephadex G-25 (LKB, Sweden).

Binding Assays of Radioactive Reagents

Following cultivation for 72 h, U937 or VTEC2.HS cells were collected, washed three times with RPMI-1640 medium, and then adjusted to a concentration of 10⁷ per ml of the same medium. Combinations of radioactive derivatives (¹²⁵I-LcrV₆₈₋₃₂₆ alone, ¹²⁵I-IFN-γ alone, ¹²⁵I-IFN-α alone, ¹²⁵I-LcrV₆₈₋₃₂₆ plus unlabled IFN-γ, and ¹²⁵I-LcrV₆₈₋₃₂₆ plus unlabeled IFN-α) were then added to individual cultures (total volume of 300 μl), which were then incubated for either 1 hr. at 4° C. or 15 min. at 37° C. Thereafter, 50 μl of the cell culture was layered on 250 μl of dibutylphthalate-bis (2-ethylhexyl)-phthalate (v/v) mixture and centrifuged for 2 min. at 14,000×g. Radioactivity in the resulting precipitate was measured using a model 1275 MINI GAMMA (LKB WALLAC, Sweden). Nonspecific binding was determined by incubation in 10,000-fold excess of corresponding unlabeled reagent. Results were expressed as the mean cpm (converted to molarity) from which nonspecific binding was subtracted. An essentially identical procedure was used to determine binding constants of LcrV and derivatives for the fragment representing the extracellular domain of TLR2.

Cytokine Determinations

Production of human IL-10 by human monocytes was measured with an ELISA kit obtained from BIOSOURCE International (USA) yielding a sensitivity of 1 pg/ml. Human TNF-α was similarly determined with according to the protocol specified for a specific ELISA kit purchased from the State Research Institute of Highly Pure Biopreparations, St. Petersburg, Russia (specificity of 1 pg/ml).

Luminol-Dependent Chemiluminescence

Production of reactive forms of oxygen by neutrophils (obtained from human blood) was evaluated by inducible chemiluminescence (van Tits et al. (2001) Free Radic. Biol. Med., 30:1122-1129). The assay mixture consisted of 400 μl of colorless Hank's balanced salt solution and Luminol (0.2 mM) either with or without LcrV. This mixture, contained in plastic tubes, was equilibrated to 37° C. within a thermostat before receiving neutrophils (2×10⁵) in 100 μl of colorless Hank's balanced salt solution. After incubation for 1 hr. to ensure adhesion to the tube walls, the reaction was initiated by addition of 100 μl of freshly opsonized (MacGregor et al. (1990) J. Gerontol., 45:M55-60) Zymosan A (Sigma) in Hank's solution (20 mg/ml). Intensity of luminescence was measured with a model 3603 Luminometer (Dialog, Moscow, Russia); Students t-test was used to evaluate the significance of the obtained results.

Results

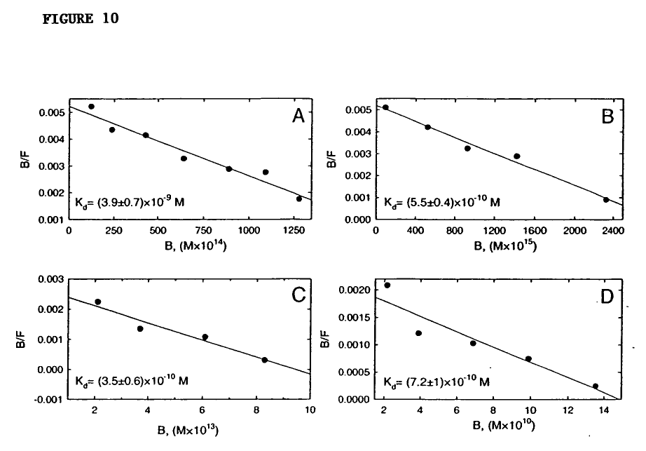

Results obtained by Scatchard analyses demonstrated that radioactive LcrV₃₁₋₅₀, LcrV₁₉₃₋₂₁₀, and LcrV₆₈₋₃₂₆ avidly bound the extracellular domain of TLR-2 at low K_{d} values of 7.2±×10⁻¹⁰ M, 3.5±0.6×10⁻¹⁰ M and 5.5±0.4×10⁻¹⁰ M, respectively. In contrast, native ¹²⁵I-LcrV exhibited a reduced K_{d} of 3.9±0.7×10⁹ M (FIG. 10).

FIG. 10 shows Scatchard analyses for specific binding of ¹²⁵I-LcrV (FIG. 10A), ¹²⁵I-LcrV₆₈₋₃₂₆ (FIG. 10B), ¹²⁵I-LcrV₁₉₃₋₂₁₀ (FIG. 10C), and ¹²⁵I-LcrV₃₁₋₅₀ to the synthetic fragment of the mouse TLR-2 extracellular domain. Molar concentrations of specifically bound radioactive LcrV and derivatives (B) are plotted as the abscissa; ratios of the bound- and free-labeled protein (B/F) constitute the ordinate. Individual specific binding constants (K_{d}) are provided.

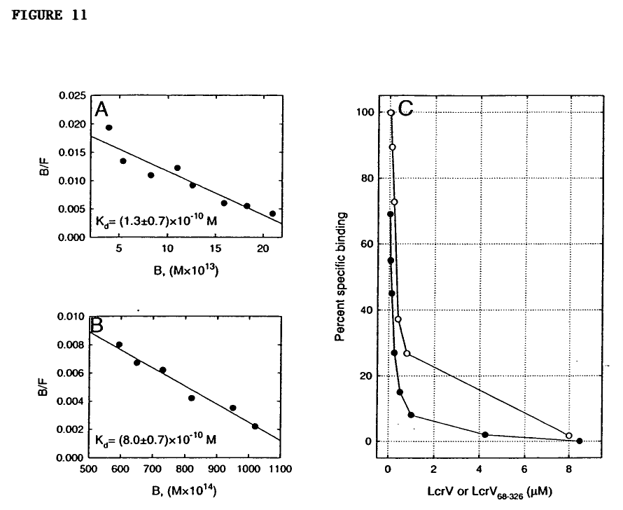

Further experiments revealed that LcrV and LcrV₆₈₋₃₂₆ successfully competed with LcrV₃₁₋₅₀ for binding to TLR-2 (data not shown) suggesting that these two structures share a second binding site at least functionally similar to that present in LcrV₃₁₋₅₀. ¹²⁵I-LcrV₃₁₋₅₀ and ¹²⁵I-LcrV₆₈₋₃₂₆ bound to the charged TLR-2 receptor of CD14-positive human thymic epithelial VTEC2.HS cells with similarly high K_{d} values of 1.3±0.7×10⁻¹⁰ M (FIG. 11A) and 8.0±0.7×10⁻¹⁰ M (FIG. 11B), respectively. In this case, binding of ¹²⁵I-LcrV was not detectable (K_{d}≧10⁻³) although unlabeled LcrV could displace TLR-2 receptor-bound ¹²⁵I-LcrV₃₁₋₅₀ but with less efficiency than did unlabeled LcrV₆₈₋₃₂₆ (FIG. 11C).

FIG. 11 shows Scatchard plot analyses of the specific binding of ¹²⁵I-LcrV₃₁₋₅₀ (FIG. 11A) and ¹²⁵I-LcrV₆₈₋₃₂₆ (FIG. 11B) to human thymic epithelial VTEC2.HS; (C) illustrates inhibition of the specific binding of ¹²⁵I-LcrV₃₁₋₅₀ to VTEC2.HS cells by unlabeled LcrV (open circles) or LcrV₆₈₋₃₂₆ (filled circles). In FIGS. 11A and 11B, molar concentrations of specifically bound radioactive LcrV derivatives (B) are plotted as the abscissa and ratios of the bound- and free-labeled protein (B/F) constitute the ordinate. Individual specific binding constants (K_{d}) are provided.

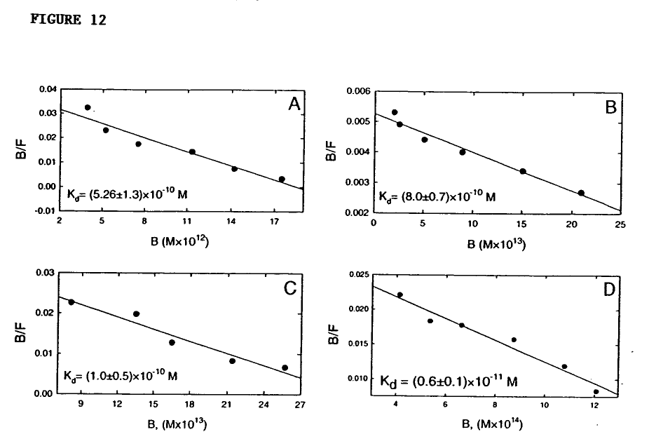

Certain CD14-negative cell lines bound LcrV and its derivatives in the absence of TLR-2. For example, LcrV, LcrV₃₁₋₅₀, and LcrV₆₈₋₃₂₆ exhibited high-affinity binding to U937 human monocytic leukemia cells provided that IFN-γ was added to the reaction. This interaction was not dependent on temperature (as judged by K_{d} values of 8.0±0.7×10⁻¹⁰ M and 1.6±0.9×10⁻¹¹ M at 4° C. and 37° C., respectively) and was essentially equivalent for LcrV₃₁₋₅₀ (FIG. 12A), LcrV₁₉₃₋₂₁₀ (FIG. 12B), LcrV₆₈₋₃₂₆ (FIG. 12C) and whole LcrV. LcrV₆₈₋₃₂₆ provided virtually identical binding to circulating professional phagocytes and cultivated thymocytes if human IFN-γ (but not IFN-α₂) was present (Table 4).

TABLE 4
Specific binding of LcrV₆₈₋₃₂₆, IFN-γ, and IFN-α₂ to receptors of U937 cells and human
neutrophils, monocytes, and thymocytes

| | Dissociation constant (Molar K_{d}) | | | | |
|---|---|---|---|---|---|
| | | | | LcrV₆₈₋₃₂₆ | LcrV₆₈₋₃₂₆ |
| Cell type | IFN-γ | IFN-α₂ | LcrV₆₈₋₃₂₆ | plus IFN-γ | plus IFN-α₂ |
| U937 | 6.8 ± 0.8 × 10⁻¹⁰ | 3.6 ± 0.4 × 10⁻¹⁰ | ≧10⁻³ | 8.0 ± 0.7 × 10⁻¹⁰ | ≧10⁻³ |
| Monocytes | 8.4 ± 0.7 × 10⁻¹⁰ | 2.1 ± 0.5 × 10⁻¹⁰ | ≧10⁻³ | 7.2 ± 0.6 × 10⁻⁹ | ≧10⁻³ |
| Neutrophils | 9.3 ± 0.8 × 10⁻¹⁰ | 8.7 ± 0.7 × 10⁻¹⁰ | ≧10⁻³ | 9.2 ± 0.9 × 10⁻¹⁰ | ≧10⁻³ |
| Thymocytes | 2.6 ± 0.4 × 10⁻¹⁰ | 5.5 ± 0.5 × 10⁻¹⁰ | ≧10⁻³ | 2.4 ± 0.3 × 10⁻⁹ | ≧10⁻³ |

¹²⁵I-LcrV bound to U937 cells at a K_{d} of 0.6±0.1×10⁻¹¹ M in the presence of the IFN-γC-terminal peptide SQMLFRGRRASQ (FIG. 12D). These findings indicate that TLR-2 receptors of CD14-positive cells are not essential for binding of LcrV to host target cells if IFN-γR-IFN-γ complexes are available.

FIG. 12 shows Scatchard plot analyses for specific binding of ¹²⁵I-LcrV (FIG. 12A), ¹²⁵I-LcrV₆₈₋₃₂₆ (FIG. 12B), and ¹²⁵I-LcrV₃₁₋₅₀ (FIG. 12C) to U937 human monocytic leukemia cells in the presence of IFN-γ. FIG. 12D shows a Scatchard plot analysis of the specific binding of ¹²⁵I-LcrV to U937 human monocytic leukemia cells in the presence of the IFN-γC-terminal peptide SQMLFRGRRASQ. Molar concentrations of specifically bound radioactive LcrV and derivatives (B) are plotted as the abscissa; ratios of the bound- and free-labeled protein (B/F) constitute the ordinate. Individual specific binding constants (K_{d}) are provided.

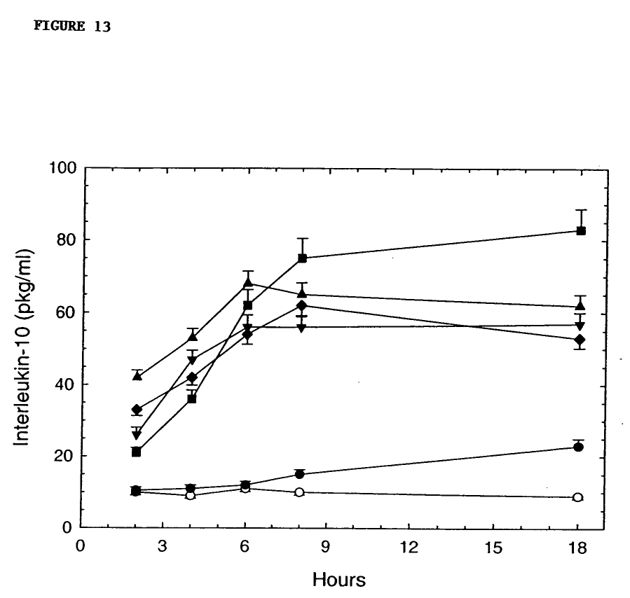

This discovery prompted evaluation of the ability of LcrV to promote synthesis of IL-10 in human blood monocytes. FIG. 13 shows a graph of the expression of IL-10 in culture supernatants of human monocytes after addition of 120 nM of LcrV (filled triangles), LcrV₆₈₋₃₂₆ (filled inverted triangles), LcrV₃₁₋₅₀ (filled diamonds), and LPS (1.0 μg/ml) provided 1 hr. before treatment with LcrV (filled squares). Controls illustrate no addition (open circles) and treatment with LPS alone (filled circles); error bars represent the standard deviation of triplicate determinations. As shown in FIG. 13, prompt upregulation of IL-10 by LcrV, LcrV₃₁₋₅₀, and LcrV₆₈₋₃₂₆ was observed in 2 hr. with maximum production occurring by 6 to 8 h. Note that the value obtained for LcrV plus LPS closely approximates the sum of those observed for LcrV alone and LPS alone.

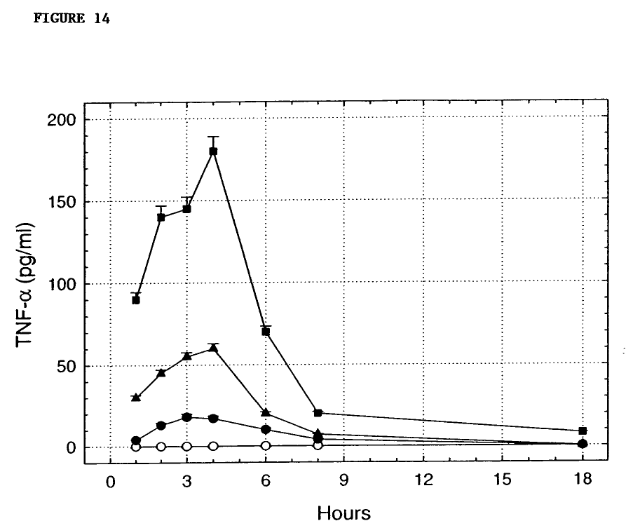

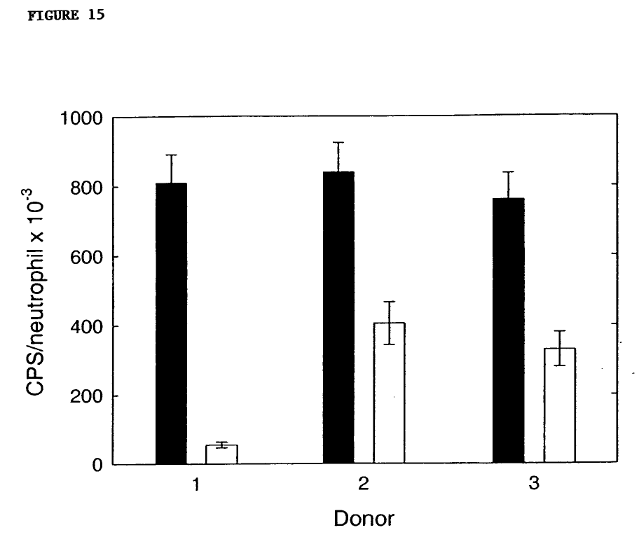

Further analysis of the role of IFN-γR-IFN-γ complexes showed that LcrV downregulated expression of TNF-α by human monocytes induced with LPS (FIG. 14) and inhibited the oxidative burst of human neutrophils (FIG. 15). FIG. 14 is a graph showing the inhibition of LPS-induced expression of TNF-α in human monocytes by LcrV. The latter were treated with LPS (100 ng/ml) for 10 min. before stimulation with LcrV (5 μg/ml); untreated monocytes (open circles), LcrV alone (filled circles), LPS plus LcrV (filled triangles), and LPS alone (filled squares). Error bars represent the standard deviation of triplicate determinations. FIG. 15 is a graph showing the inhibition of Luminol-dependent chemiluminescence of neutrophils (2×10⁵/ml) from normal human donors cultivated at 37° C. for 1 hr. in the absence (open bar) or presence (closed bar) of LcrV (120 nM); the oxidative burst was initiated by addition of 100 μl of a suspension of Zymosan A (20 mg/ml Hank's solution) per ml of neutrophil culture. Error bars represent the standard deviation of triplicate determinations.

Discussion

Considered together, these findings indicate that LcrV possesses at least two domains capable of upregulating IL-10 that, in turn, eliminates nuclear NF-κB thereby downregulating numerous inflammatory effectors (Moore et al. (2001) Ann. Rev. Immunol., 19:683-765) required for containment of Yersinia pestis . Inspection of LcrV₃₁₋₅₀ as well as the biologically active N-terminal sequences V5, V7, and V9 of Sing et al. (Sing et al. (2002) J. Exp. Med., 196:1017-1024) reveals a motif consisting of two acidic amino acids surrounded by hydrophobic residues (LEEL) at position 32 to 35; a similar motif (DEEI) comprises amino acids 203 to 206 in LcrV₁₉₃₋₂₁₀, LcrV₆₈₋₃₂₆, and LcrV. The two glutamic acid residues of these motifs are capable of electrostatic interaction with adjacent basic arginine residues in the IRRL sequence of the extracellular domain of TLR-2 and the GRRA sequence of the C-terminus of human IFN-γ. The surrounding hydrophobic amino acids in these motifs enhance attraction of the charged clusters thus promoting the strong binding that was observed. In this context, only receptor-bound IFN-γ possesses sufficient ordered secondary structure at the C-terminus to predict significant binding with LcrV (Walter et al. (1995) Biochem., 34:12118-12125). The LEEL motif of LcrV₃₁₋₅₀ is located within the sterically inaccessible α-helix 1 of LcrV whereas the DEEI sequence of LcrV₁₉₃₋₂₁₀ and LcrV₆₈₋₃₂₆ resides within exposed α-helix 9 and is movably connected to the molecular surface via two unstructured hydrophobic sequences (Derewenda et al. (2004) Structure, 12:301-306).

These results show that the observed binding of TLR-2 and IFN-γ occurs at the LEEL site of LcrV₃₁₋₅₀ and at the DEEI site of LcrV₁₉₃₋₂₁₀ and LcrV₆₈₋₃₂₆ and that, as judged by K_{d} values and steric constraints, binding is not cooperative and only the motif shared by LcrV₁₉₃₋₂₁₀ and LcrV₆₈₋₃₂₆ functions in native LcrV. Five amino acids separate both the LEEL and DEEI binding sites from DKN residues. These 3 amino acids may initiate events leading to upregulation of IL-10. LcrV lacking the N-terminal LEEL site and attendant DKN residue provided excellent protection as opposed to LcrV containing a more C-terminal deletion at amino acids 241 to 270 (Overheim et al. (2005) Infect. limun., 73:5152-5159).

As described above, LcrV is an integral component of the pCD-encoded TTSS injectisome, targets delivery of cytotoxic Yops, and downregulates inflammatory host effectors. Neutralization of any one of these processes could account for the remarkable immunity provided by anti-LcrV. Accordingly, anti-LcrV may immunize by blocking type III secretion. LcrV may therefore fulfill an additional role in pathogenesis by serving as a surface adhesin promoting close bacterium-host cell contact necessary for Yop translocation. As already noted, LcrV exists on the bacterial surface and results presented here clearly place the protein on the host cell in association with TLR-2 and IFN-γ receptors. Attempts to determine the molecular weight of soluble LcrV by molecular sieving indicated that the molecule forms a stable dimer under physiological conditions (Brubaker et al. (1987) Microb. Pathogen, 2:49-62). Further study may show that LcrV bound to TLR-2 and IFN-γ receptors forms similar dimers with that on the bacterial surface thereby promoting the adhesion necessary for targeting the translocation of Yops. Accordingly, disruption of this binding could provide the basis for anti-LcrV immunity.

### 5.3 Example 3

### Promoting Allograft Retention and Wound Healing

As demonstrated above, the conserved TLR2 and IFN-γR-IFN-γ-binding sites of LcrV serve to activate TLR2 and upregulate the major host anti-inflammatory cytokines including interleukin-10 (IL-10) which, in turn, blocks the ability of host nuclear NF-kB to activate transcription of a plethora of inflammatory activities including proinflammatory cytokines. The latter are necessary for activation of phagocytes and formation of protective granulomas that serve to contain invading yersiniae. This observation has relevance to vaccine production because antibodies directed against LcrV block upregulation of IL-10 and thus downregulate proinflammatory cytokines, inhibit formation of protective granulomas, and protect against disease. LcrV from yersiniae actively upregulates IL-10 thus making it a formidable therapeutic agent in certain applications outside where immunosuppression is desirable.

In order to determine the level at which LcrV functions to block innate immunity as well as specific immunity, its effect on allograft retention was examined. Skin was grafted from an inbred black mouse strain onto its white counterpart and the effect of injected LcrV on allograft rejection was examined.

The results showed that LcrV had no effect on specific immunity but very effectively blocked the general inflammation associated with the grafting process in mice injected daily with a minimal amount of homogenous LcrV. In particular, all normal erythema and edema associated with the trauma of skin patching vanished completely in treated mice and the grafts set without any sign of inflammation. Both LcrV treated and untreated allograft groups underwent rejection at the same time but this process began early and was extended for many days in the controls relative to the treated group, where it was postponed but then occurred rapidly (for further detail, see Motin et al. (1997) Transplantation 63:1040-2). Accordingly, LcrV blocks inflammation but not specific immunity and therefore that it clearly facilitates wound-healing and thereby minimizes scarring and facilitates healing in applications such as burn therapy, pre-surgical procedure, and other wound healing applications known in the art.

### 5.4 Example 4

### HIV Treatment

Because LcrV indirectly inactivates NF-κB, which is a transcriptional activator that is required for HIV replication, LcrV protein and related TLR2 and IFN-γR-IFN-γ-binding LcrV proteins and polypeptides is used to treat HIV as well as other viral infections that require host NF-κB activity. Accordingly, the effects of LcrV polyptides on HIV replication in tissue culture are examined and the results indicate that HIV replication is inhibited. Further, preparations of LcrV are administered to a host infected with, or at risk for infection with, HIV and anti-viral effects are examined.

### 5.5. Example 5

### Cancer Therapy

Studies indicate that there is also a link between expression of NF-κB and cancer (Marx (2004) Science 306: 966-968). The relationship reflects the ability of malignant cells to upregulate this transcriptional activator thereby assuring expression of inflammatory functions that protect against apoptosis and vascularize tumors. Accordingly, LcrV protein and related TLR2 and IFN-γR-IFN-γ-binding LcrV proteins and polypeptides are used to inactivate NF-κB and thereby prevent tumor development and treat cancer. This was proven in two experiments where inbred mice were injected with an avirulent guanine auxotroph (gua) of Y. pestis capable of prolonged synthesis of LcrV in vivo and then with mouse tumor cells (malignant melanoma). All mice in the control group died of cancer, whereas about 90% of the treated group survived.

C57B6 mice are injected with 5×10⁶ B16F10 melanoma tumor cells followed by intraperitoneal injections for six days of PBS alone or 10 to 100 μg of purified Y. pestis LcrV immunogenic polypeptide, starting on post challenge day four. PBS treated control mice develop palpable tumors that become extensive by the second week whereupon death commences. In contrast the mice injected LcrV immunogenic polypeptide show markedly reduced morbidity (80% five weeks after initial challenge). Accordingly these results indicate that LcrV immunogenic polypeptide is effective in treating cancer.

In further experiments, homogenous purified LcrV protein and polypeptides are administered in place of the gua auxotroph to downregulate inflammation and thereby prevent tumor cell vascularization and metastasis.

Equivalents

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A polypeptide comprising an immunogenic Yersinia V-antigen consensus polypeptide consisting essentially of VLEELXXXXX DKN (SEQ ID NO: 32).

2. A polypeptide comprising an immunogenic Yersinia V-antigen consensus polypeptide consisting essentially of DKNXXX XTDEEIF (SEQ ID NO: 33).

3. An immunogenic polypeptide mixture comprising at least one polypeptide having an immunogenic Yersinia V-antigen consensus polypeptide consisting essentially of VLEELXXXXX DKN (SEQ ID NO: 32); and at least one polypeptide having an immunogenic Yersinia V-antigen consensus polypeptide consisting essentially of DKNXXX XTDEEIF (SEQ ID NO: 33).

4. An immunogenic polypeptide conjugate comprising a polypeptide of claim 1 or claim 2, linked to a carrier.

5. A Yersinia vaccine comprising an immunogenic polypeptide or immunogenic polypeptide mixture of any of claims 1-4.

6. The vaccine of claim 5, further comprising an adjuvant.

7. The vaccine of claim 5, further comprising a protein carrier.

8. The vaccine of claim 6, wherein the adjuvant is selected from the group: alum, a polymer adjuvant, a co-polymer adjuvant, Freund's complete adjuvant, Freund's incomplete adjuvant, sorbitan monooleate, QS 21, muramyl dipeptide, a CpG oligonucleotide adjuvant, trehalose, a bacterial extract adjuvant, a detoxified endotoxin adjuvant, a membrane lipid adjuvant, and combinations thereof.

9. A method of treating or preventing a Yersinia infection in a mammal comprising administering to the mammal an immunogenic amount of a polypeptide of any of claims 1-4.

10. A method of treating or preventing a Yersinia infection in a mammal comprising administering to the mammal an immunogenic amount of a vaccine of claim 5.

11. A method of treating or preventing a Yersinia infection in a mammal comprising administering to the mammal an immunogenic amount of a vaccine of claim 8.

12. The method of claim 10, wherein the mammal is a human.

13. The method of claim 11, wherein the mammal is a human.

14. The method of claim 9, further comprising collecting immune serum from the mammal and administering the immune serum to a second mammal in need thereof.

15. The method of claim 10, further comprising collecting immune serum from the mammal and administering the immune serum to a second mammal in need thereof.

16. The method of claim 14, wherein the second mammal is a human.

17. The method of claim 15, wherein the second mammal is a human.

18. A method of screening for a Yersinia infection immunomodulatory compound comprising:
contacting a V-antigen binding unit comprising LcrV, or a polypeptide comprising a Yersinia V-antigen consensus polypeptide consisting essentially of VLEELXXXXX DKN (SEQ ID NO: 32) or DKNXXX XTDEEIF (SEQ ID NO: 33), with an interferon gamma receptor/interferon gamma ligand ternary complex (IFN-γR-IFN-γ) in the presence of a test compound;
measuring the amount of binding of the V-antigen binding unit to the interferon gamma receptor/interferon gamma ligand ternary complex (IFN-γR-IFN-γ) in the presence of the test compound; and
comparing the amount of binding of the V-antigen binding unit to the interferon gamma receptor/interferon gamma ligand ternary complex (IFN-γR-IFN-γ) in the presence of the test compound with the amount of binding of the V-antigen binding unit to the interferon gamma receptor/interferon gamma ligand ternary complex (IFN-γR-IFN-γ) in the absence of the test compound,
wherein the compound is a Yersinia infection immunomodulatory compound if the amount of binding in the presence of the test compound is less than the amount of binding in the absence of the test compound.

19. The method of claim 18, wherein the interferon gamma receptor/interferon gamma ligand ternary complex (IFN-γR-IFN-γ) is expressed on the surface of a CD14-negative cell.

20. The method of claim 19, wherein the CD14-negative cell is selected from the group consisting of a human monocyte and a human neutrophilic leukocyte.
